(19) United States
(12) Patent Application Publication
(10) Pub. No.: US 2023/0270764 A1
(43) Pub. Date: Aug. 31, 2023

(54) COMPOSITION COMPRISING CANNABINOIDS, TERPENES, AND FLAVONOIDS FOR TREATING DEPRESSION

(71) Applicant: Raz YIRMIYA, Ramat Gan (IL)

(72) Inventor: Raz YIRMIYA, Ramat Gan (IL)

(21) Appl. No.: 18/015,790

(22) PCT Filed: Jul. 14, 2021
(86) PCT No.: PCT/IL2021/050865

§ 371 Date: Jan. 12, 2023

Related U.S. Application Data
(60) Provisional application No. 63/051,445, filed on Jul. 14, 2020.

(51) Int. Cl. A61K 31/00; A61K 31/415; A61P 37/00; A61P 29/00; A61P 25/16; A61P 25/28; A61K 31/015; A61K 31/01; A61K 31/353; A61K 31/09; A61K 31/7048; A61K 31/575; A61K 31/045; A61K 31/352; A61P 25/24; A61P 25/22

(52) U.S. Cl. CPC A61K 31/658; A61K 31/01; A61K 31/015; A61K 31/045; A61K 31/09; A61K 31/352; A61K 31/353; A61K 31/415; A61K 31/575; A61K 31/7048; A61P 25/16; A61P 25/22; A61P 25/24; A61P 25/28; A61P 29/00; A61P 37/00

(57) ABSTRACT

The present invention is directed to a pharmaceutical composition, including a method of using same, such as for treating or attenuating a mood disorder in a subject having a high inflammatory state. The pharmaceutical composition and the method of the invention, in some embodiments thereof, comprise suppressing or inhibiting microglia for treating a mood disorder.

### CROSS-REFERENCE TO RELATED APPLICATIONS

This application claims the benefit of priority of U.S. Provisional Pat. Application No. 63/051,445, titled “COMPOSITION COMPRISING CANNABINOIDS, TERPENES, AND FLAVONOIDS FOR TREATING DEPRESSION”, filed Jul. 14, 2020, the contents of which are incorporated herein by reference in their entirety.

### FIELD OF INVENTION

The present invention is directed to, inter alia, pharmaceutical compositions and methods of using the same, in treating mood disorders, e.g., depression, by reducing the levels of inflammation and brain microglia activation.

### BACKGROUND

Despite impressive progress in understanding the molecular, cellular, and circuit-level correlates of major depression, the biological mechanisms that causally underlie this disease are still unclear, hindering the development of effective preventive and therapeutic procedures. One possible reason for this situation is that almost all research in this area focuses on the involvement of abnormalities in neuronal functioning, whereas the involvement of non-neuronal brain cells was not thoroughly examined. Over the last decade it has been shown that inflammatory processes in the immune system, in general, and changes in brain microglia cells, in particular, are also involved in the development of depression.

The role of microglia in depression has been suggested by the following lines of evidence: (1) Microglial activation status is increased in some depressed patients (particularly in relation to suicide); (2) Most neurological diseases of the brain are characterized by a high incidence of depression along with increased microglial activation status; (3) Pro-inflammatory cytokines, whose main source in the brain is microglia, are elevated in the cerebro-spinal fluid and plasma of depressed patients; (4) A substantial percentage of cancer or hepatitis C patients who receive immunotherapy with IFN-α, which activates microglia, develop major depression; (5) Healthy volunteers that are experimentally exposed to microglia stimulators, particularly endotoxin (lipopolysaccharides (LPS)), develop depressed mood; (6) Exposure to various forms of stress in experimental animals induces microglia activation along with the development of depressive-like symptoms; (7) Antidepressant drugs modulate microglial activities; and (8) Microglia activation decreases hippocampal neurogenesis, which is an important mechanism for the development of depression and a major target for antidepressant drugs.

Anxiety is often apparent in depressed patients as well as in animal models of acute-and chronic stress-induced depression, and this condition is also influenced by microglia. For example, microglial activation has been noted in many animal models in which anxiety is a major symptom. Furthermore, genetic alterations in important microglia-related genes can modulate anxiety levels and their responsiveness to external triggers. In such models, treatment with microglia suppressive drugs, such as minocycline, could attenuate or completely block the development and/or manifestations of anxiety.

Ample evidence demonstrates that microglia changes in depressed patients and in experimentally-induced depression/anxiety in animals do not always comprise microglia activation processes, and under some circumstances depression can be caused by decline and degeneration of microglia. Together, these findings indicate the need for a personalized treatment approach, based on the microglial/inflammatory status of the individual depressed patient.

Previous research demonstrated that some strains of the cannabis plant and some pure molecules derived from cannabis have anti-inflammatory effects. Pure cannabis components, including cannabidiol (CBD), Δ9-tetrahydrocannabinol (THC), cannabigerolic acid (CBGA), β-caryophyllene (BCP), linalool, β-elemene, luteolin, kaempferol, quercetin and apigenin, were also shown individually to inhibit microglia activation.

### SUMMARY

The present invention, in some embodiments thereof, relates to methods and compositions for treating a mood disorder in a subject in need thereof. In some embodiments, there is provided a microglia suppressive formulation based on a composition of a first cannabinoid, and at least one additional therapeutic compound selected from: at least one cannabinoid being different from the first cannabinoid, a terpene, a flavonoid, and any combination thereof.

In some embodiments, the herein disclosed cannabis-related formulation is highly effective in a subject afflicted with depression. In some embodiments, the subject afflicted with depression is characterized as having activated microglial inflammatory status. In some embodiments, the composition of the invention reduces or eliminates antidepressant treatment failure and/or adverse effects in a subject afflicted with depression.

According to a first aspect, there is provided method for treating or attenuating a mood disorder in a subject in need thereof, the method comprising: (a) determining whether the subject has a high inflammatory state and/or microglial activation state; and (b) administering to the subject determined as having a high inflammatory state and/or microglial activation state a (i) a therapeutically effective amount of a first cannabinoid, and (ii) a therapeutically effective amount of at least one additional therapeutic compound, thereby treating or attenuating a mood disorder in the subject.

According to another aspect, there is provided pharmaceutical composition comprising a first cannabinoid, at least one additional therapeutic compound, and a pharmaceutically acceptable carrier, for use in treating a mood disorder in a subject in need thereof, and being characterized by having microglia suppressive activity.

In some embodiments, the high inflammatory/microglia state, is characterized by any one of: (a) plasma C-reactive protein (CRP) levels greater than 1 mg/L; (b) plasma IL-1β levels greater than 60 pg/ml; (c) plasma IL-6 levels greater than 2.0 pg/ml; (d) plasma TNFα levels greater than 3.8 pg/ml; (e) plasma CCL11 (eotaxin-1) levels greater than 72 pg/ml; (g) plasma IL-8 levels greater than 12.0 pg/ml; (h) plasma MIP-1α greater than 7.1 pg/ml; (i) plasma MCP1 levels greater than 80.0 pg/ml, (j) plasma RANTES greater than 2978 pg/ml, and any combination thereof.

In some embodiments, the determining is determining in a sample derived from the subject.

In some embodiments, the method further comprises providing a sample from the subject and performing the determining in the sample.

In some embodiments, the high inflammatory state and/or microglial activation state comprises microglia activation.

In some embodiments, the subject is resistant to standard medication for treatment of the condition.

In some embodiments, the mood disorder is selected from the group consisting of: major depressive disorder, unipolar major depressive episode, dysthymic disorder, treatment-resistant depression, bipolar depression, adjustment disorder with depressive mood, cyclothymic disorder, atypical depression, depression associated with REM sleep behavior disorder, seasonal affective disorder, depression co-morbid with anxiety disorder, generalized anxiety disorder, melancholic depression, psychotic depression, post-schizophrenic depression, schizophrenia, depression due to a general medical condition, depression associated with a synucleinopathy, Parkinson’s disease, multiple system atrophy (MSA), depression associated with autosomal-recessive Parkinson’s disease, depression associated with the diffuse/malignant subtype of Parkinson’s disease, depression associated with Alzheimer’s disease, depression associated with multiple sclerosis, depression associated with Tourette’s disorder, depression associated with dystonia, depression associated with ataxia, depression associated with dyskinesia, depression associated with essential tremor, depression associated with PTSD, post-viral fatigue syndrome, chronic fatigue syndrome, depression associated with autism spectrum disorder, depression associated with schizophrenia, depression associated with somatoform disorder, depression associated with somatic symptom disorder, depression associated with pain disorder, depression associated with rheumatoid arthritis, depression associated with osteoarthritis, depression associated with ankylosing spondylitis, depression associated with lupus erythematosus, depression associated with Crohn’s disease, depression associated with inflammatory bowel disease, depression associated with Williams syndrome, depression associated with the DiGeorge syndrome, depression associated with cancer, depression associated with primary biliary cholangitis, depression associated with autoimmune hepatitis, and depression associated with neurofibromatosis, and fibromyalgia.

In some embodiments, the at least one additional therapeutic compound is selected from the group consisting of: at least one cannabinoid being different from the first cannabinoid, a terpene, a flavonoid, and any combination thereof.

In some embodiments, the any one of first cannabinoid and the at least one cannabinoid being different from said first cannabinoid is selected from the group consisting of: cannabidiol (CBD), Δ-9-tetrahydrocannabinol (Δ-9-THC), Δ-8-tetrahydrocannabinol (Δ-8-THC), cannabigerol (CBG), cannabichromene (CBC), cannabigerolic acid (CBGA), Cannabidiolic acid (CBDA), THC acid (THCA), cannabichromenic Acid (CBCA), Cannabidivarin (CBDV), tetrahydrocannabivarin (THCV), Cannabichromevarin (CBCV), Cannabichromerocin (CBCV), Cannabivarin (CBV), Cannabicitran (CBT), Cannabinol (CBN), Cannabicyclol (CBL), Cannabigerorcin (CBGO), and Cannabinodiol (CBND).

In some embodiments, the first cannabinoid is CBD.

In some embodiments, the flavonoid is selected from the group consisting of: kaempferol, quercetin, cannflavin A, cannflavin B, canniprene, luteolin, apigenin, orientin, β-sitosterol, vitexin, isovitexin, and chrysin.

In some embodiments, the terpene is selected from the group consisting of: β-caryophyllene, β-myrcene, linalool, α-pinene, β-pinene, limonene, β-amyrin, eucalyptol, alpha-terpineol, valencene, geraniol (lemonol), β-elemene, bisabolol, nerolidol, ocimene, terpinolene, humulene, α-terpinene, camphene, fenchol, α-phellandrene, Δ3-carene, γ-cardinene, sabinene, and cycloartenol.

In some embodiments, the at least one additional therapeutic compound is an anti-inflammatory drug, an anti-microglial drug, or a combination thereof.

In some embodiments, the anti-inflammatory drug is a non-steroidal anti-inflammatory drug (NSAID).

In some embodiments, the NSAID is selected from the group consisting of: celecoxib, aspirin, diclofenac, diflunisal, etodolac, ibuprofen, indomethacin, and acetaminophen.

In some embodiments, the NSAID is celecoxib.

In some embodiments, the at least one additional therapeutic compound is administered simultaneously or sequentially.

In some embodiments, the method further comprises applying to the subject an anti-inflammatory or an anti-microglial therapy.

In some embodiments, the anti-inflammatory therapy or said anti-microglial therapy comprises electroconvulsive therapy (ECT).

In some embodiments, the subject is determined as having a high inflammatory state and/or microglial activation state.

In some embodiments, the NSAID is celecoxib.

In some embodiments, the pharmaceutical composition comprises CBD and kaempferol, at a ratio ranging from 4:1 to 1:4.

In some embodiments, the pharmaceutical composition comprises CBD and quercetin, at a ratio ranging from 4:1 to 1:4.

In some embodiments, the pharmaceutical composition comprises CBD and kaempferol, and quercetin, at a ratio ranging from 4:1:1 to 1:4:4.

In some embodiments, the pharmaceutical composition comprises CBD and β-caryophyllene, and kaempferol, at a ratio ranging from 4:1:1 to 1:4:4.

In some embodiments, the pharmaceutical composition comprises CBD and β-caryophyllene, and quercetin, at a ratio ranging from 4:1:1 to 1:4:4.

In some embodiments, the pharmaceutical composition comprises CBD and THC, at a ratio ranging from 200:1 to 1:1.

In some embodiments, the pharmaceutical composition comprises CBD, THC, and kaempferol, at a ratio ranging from 200:1:400 to 1:1:4.

In some embodiments, the pharmaceutical composition comprises CBD, THC, and quercetin, at a ratio ranging from 200:1:400 to 1:1:4.

In some embodiments, the pharmaceutical composition comprises CBD and celecoxib, at a ratio ranging from 4:1 to 1:4.

In some embodiments, the pharmaceutical composition comprises a whole plant cannabis extract comprising CBD and THC at a ratio ranging from 30:1 (w/w) to 1:1 (w/w).

In some embodiments, the pharmaceutical composition comprises a whole plant cannabis extract comprising CBD and THC at a ratio ranging from 30:1 (w/w) to 1:1 (w/w), and kaempferol.

In some embodiments, the pharmaceutical composition comprises a whole plant cannabis extract comprising CBD and THC at a ratio ranging from 30:1 (w/w) to 1:1 (w/w), and quercetin.

In some embodiments, the pharmaceutical composition comprises a whole plant cannabis extract comprising CBD and THC at a ratio ranging from 30:1 (w/w) to 1:1 (w/w), and β-caryophellene.

In some embodiments, the pharmaceutical composition comprises a whole plant cannabis extract comprising CBD and THC at a ratio ranging from 30:1 (w/w) to 1:1 (w/w) and β-caryophellene, and kaempferol, and quercetin.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 is a graph depicting the involvement of different microglia states in brain and mental health and disease. Homeostatic microglia state is important for mental health, while deviations from this state (including either hypoactivity or hyperactivity of microglia) are associated with depression. The current invention establishes the utility of a personalized medical approach to depression, based on treating or attenuating depression in a subject having a high inflammatory state and/or microglial activation state, as determined by specific brain and plasma biomarkers.

FIGS. 2A-2B are schemes depicting the general approach for identifying specific formulations of a cannabinoid(s) with terpene(s) and/or a flavonoid(s) and/or celecoxib (CLX; 2A), and a whole plant cannabis extract supplemented by with a specific terpene(s) and/or a flavonoid(s) and/or CLX (2B), which produce the most potent suppressive effects in LPS or poly I:C or α-synuclein or β-amyloid-induced microglia activation.

FIGS. 3A-3B are vertical bar graphs describing the effects of selected cannabinoids, terpenes and flavonoids and their combinations on lipopolysaccharide-induced TNFα secretion by BV2 microglia cell cultures. As shown in (3A) cannabidiol (CBD, at 10, but not 1 µM) inhibited TNFα production, whereas relatively low doses of Δ-9 tetrahydrocannabinol (THC, at 0.1, 0.5 and 1 µM) had no effect. The cannabinoids: Cannabidiolic acid (CBDA, at 0.2, 1 and 2 µM) and cannabichromene (CBC, at 0.1, 1 and 2 µM) also had no effect. As shown in (3B) the flavonoids kaempferol (Kae, at 10, 25 and 50 µM) and quercetin (Quer, at 10 and 25, but not 5 µM) exerted a potent inhibitory effect. The terpenes α-pinene, β-pinene, and limonene (at either 25 or 100 µM) produced no effects. The terpene linalool (at 100 but not 25 µM) produced a small inhibitory effect. A combination of CBD (5 µM) and quercetin (10 µM) produced a synergistic effect, compared with the effects of CBD or quercetin by themselves. An even greater synergistic effect was obtained by treatment with a combination of CBD (5 µM), THC (0.1 µM) and quercetin (10 µM).

FIGS. 4A-4F are vertical bar graphs describing the effects of CBD (at concentrations of 1 - 10 µM) in in vitro cellular models of neuroinflammation induced by the bacterial endotoxin (lipopolysaccharides, LPS) or viral mimetic poly I:C or an aggregated form of α-synuclein. Accumulation of α-synuclein aggregates and fibrils leads to many pathological alterations, including excessive microglia activation, in Parkinson’s disease and other synucleinopathies. As shown in (4A and 4B), 10 µM of CBD (but not 1, 2.5 or 5 µM) significantly decreased the release of TNFα and IL-6 proteins, while 5 and 10 µM of CBD significantly reduced the release of IL-1β protein from LPS-activated microglia (4C). As shown in (4D), in BV2 microglia activated by Poly I:C, 5 µM and 10 µM of CBD reduced significantly the release of TNFα protein, while 10 µM reduced efficiently also IL-6 protein release (4E). As shown in (4F), in BV2 microglia activated by α-synuclein, 10 µM of CBD significantly decreased the release of TNFα protein. *p<0.05 compared with vehicle (Veh)-treated cultures.

FIGS. 5A-5D are vertical bar graphs describing the effects of the cannabinoid CBD and the flavonoid kaempferol (each at concentrations of 1, 2.5 and 5 µM), as well as of combinations of CBD with kaempferol, on the release of TNFα and IL-1β from LPS-activated BV2 microglial cells. As shown in (5A), whereas the tested concentrations of CBD or kaempferol by themselves had no effects, combinations of 5 µM CBD plus either 2.5 or 5 µM kaempferol significantly suppressed the release of TNFα, as compared with the effect observed in LPS-activated cultures treated with vehicle (Veh). Furthermore, several combinations of CBD plus kaempferol produced a significant suppression of TNFα secretion compared with the individual components given by themselves. As shown in (5B), neither 1 µM nor 2.5 µM of CBD as well as of kaempferol affected the levels of IL-1β protein released from LPS-activated microglia. However, several combinations of both compounds at these individually inactive doses significantly lowered the release of IL-1β as compared to the levels observed in LPS-activated cultures treated with vehicle (Veh). Moreover, although 5 µM of CBD as well as 5 µM of kaempferol (given separately) significantly suppressed IL-1β levels, the combination of both compounds in these doses resulted in even stronger decrease in IL-1β levels as compared to each compound given individually. *p<0.05 compared with LPS-exposed cultures treated with Veh. ^{$}p<0.05 compared with cultured treated with the corresponding concentration of CBD by itself. ^{#}p<0.05 compared with cultured treated with the corresponding concentration of kaempferol by itself. FIGS. 5C-5D are concentration-response plots, showing nonlinear regression curves fitted based on wide-range concentration-response experiments of the TNFα and IL-1β suppressive effects of the various compounds and combinations. Based on these plots, the IC50 values were computed, defined as the concentrations of CBD and kaempferol, by themselves or in combinations, at which the response to LPS is reduced by half of the maximal effect. The Table on the right of each XY plot demonstrates the markedly lower IC50 concentrations of combinations of CBD plus kaempferol, as compared with the IC50 of each compound given by itself.

FIGS. 6A-6D are vertical bar graphs describing the effects of the cannabinoid CBD and the flavonoid quercetin (each at concentrations of 1, 2.5 and 5 µM), as well as of combinations of CBD with quercetin, on the release of TNFα and IL-1β from LPS-activated BV2 microglial cells. As shown in (6A), when applied by themselves, CBD (1 and 2.5 µM) and quercetin (1, 2.5 and 5 µM) did not affect the release of TNFα by LPS-activated microglia, as compared with the effect observed in LPS-activated cultures treated with vehicle (Veh). CBD at 5 µM decreased significantly the TNFα cytokine release from LPS-activated BV2 cells. Combinations of inactive concentrations of CBD (1 or 2.5 µM) with inactive concentrations of flavonoid quercetin (1 or 2.5 or 5 µM) resulted in significantly stronger inhibition of TNFα as compared with the levels detected in LPS-activated microglial cells treated with Veh, with one combination (CBD (2.5 µM) plus quercetin (1 µM) exhibiting significantly stronger effect than each compound given individually. As shown in (6B), neither CBD nor quercetin (each at 1 or 2.5 µM) given individually affected the release of IL-1β protein by LPS-activated BV2 cells, as compared with the effect observed in LPS-activated cultures treated with vehicle (Veh). In contrast, CBD or quercetin (5 µM) by themselves significantly reduced the release of IL-1β. Combinations of CBD (2.5 or 5 µM) with inactive concentrations of quercetin (1 or 2.5 µM) potentiated the IL-1β inhibition as compared to the LPS-activated microglial cells treated with Veh, with several combinations markedly stronger than the effect of each compound tested individually at the respective dose. *p<0.05 compared with LPS-exposed cultures treated with Veh. ^{$}p<0.05 compared with cultured treated with the corresponding concentration of CBD by itself. ^{#}p<0.05 compared with cultured treated with the corresponding concentration of quercetin by itself. (6C-6D) Are concentration-response plots, showing nonlinear regression curves fitted based on wide-range concentration-response experiments of the TNFα (6C) and IL-1β (6D) suppressive effects of the various compounds and combinations. Based on these plots, the IC50 values were computed, defined as the concentrations of CBD and quercetin, by themselves or in combinations, at which the response to LPS is reduced by half of the maximal effect. The Table on the right of each XY plot demonstrates the markedly lower IC50 concentrations of combinations of CBD plus quercetin, as compared with the IC50 of each compound given by itself.

FIGS. 7A-7D are vertical bar graphs describing the effects of the cannabinoids CBD (concentrations of 1, 2.5 and 5 µM) and cannabidivarin (CBDV; at concentrations of 1, 2.5, 5 and 10 µM), as well as of combinations of CBD with CBDV, on the release of TNFα and IL-6 from LPS-activated BV2 microglial cells. As shown in (7A), no concentration of either CBD or CBDV by itself affected release of TNFα, as compared with the effect observed in LPS-activated cultures treated with vehicle (Veh). Combination of CBD (5 µM) plus CBDV (at 1, 5 or 10 µM) significantly enhanced the inhibition of the TNFα cytokine release from LPS-activated BV2 cells, as compared to Veh plus LPS treated cells. As shown in (7B), no concentration of either CBD or CBDV by itself affected release of IL-6, as compared with the effect observed in LPS-activated cultures treated with vehicle (Veh). Combinations of CBDV (10 µM) with each the tested concentrations of CBD (1, 2.5 or 5 µM) significantly enhanced the IL-6 inhibition from LPS-activated cells. Lower concentrations of CBDV (1, 2.5 or 5 µM) also enhanced the effect of CBD at 2.5 µM and at 5 µM. Several of these combinations were significantly stronger that the effects observed for each compound tested individually at the corresponding concentration. *p<0.05 compared with LPS-exposed cultures treated with Veh. ^{$}p<0.05 compared with cultured treated with the corresponding concentration of CBD by itself. ^{#}p<0.05 compared with cultured treated with the corresponding concentration of CBDV by itself. (7C-7D) Are concentration-response plots, showing nonlinear regression curves fitted based on wide-range concentration-response experiments of the TNFα and IL-6 suppressive effects of the various compounds and combinations. Based on these plots, the IC50 values were computed, defined as the concentrations of CBD and CBDV, by themselves or in combinations, at which the response to LPS is reduced by half of the maximal effect. The Table on the right of each XY plot demonstrates the markedly lower IC50 concentrations of combinations of CBD plus CBDV, as compared with the IC50 of each compound given by itself.

FIGS. 8A-8D depict the effects of CLX, CBD and their combinations on LPS-induced pro-inflammatory cytokines secretion by BV2 microglia cell cultures. (8A-8B) Are vertical bar graphs showing the percent inhibition of LPS-induced TNFα and IL-1β secretion exerted by the various compounds and their combinations. As shown, CLX or CBD by themselves did not produced significant TNFα or IL-1β inhibitory effects (besides the higher concentration of CBD, which did suppress IL-1β secretion). Combinations of 5 µM CBD with CLX (at 2.5, 5 or 10 µM for TNFα, and at all concentrations for IL-1β), as well as combinations of 10 µM CLX with either 1 or 2.5 µM CBD, produced significant TNFα- and IL-1β-inhibitory effects, compared with the LPS-exposed cultures treated with vehicle (Veh) values. Furthermore, combinations of 10 µM CLX with low concentrations of CBD produced a significant inhibitory effect for both cytokines, compared with the effects of CBD by itself. *p<0.05 compared with LPS-exposed cultures treated with Veh. ^{#}p<0.05 compared with cultured treated with the corresponding concentration of CLX by itself. ^{$}p<0.05 compared with cultured treated with the corresponding concentration of CBD by itself. (8C-8D) Are concentration-response plots, showing nonlinear regression curves fitted based on wide-range concentration-response experiments of the TNFα (8C) and IL-1β (8D) suppressive effects of the various compounds and combinations. Based on these plots, the IC50 values were computed, defined as the concentrations of CLX and CBD, by themselves or in combinations, at which the response to LPS is reduced by half of the maximal effect. The Table on the right of each XY plot demonstrates the markedly lower IC50 concentrations of combinations of CLX plus CBD, as compared with the IC50 of each compound given by itself.

FIGS. 9A-9B depict the effects of various concentrations of CLX and CBD in a model of neuroinflammation in Parkinson’s disease, i.e., α-Synuclein-induced TNFα secretion by BV2 microglia cell cultures. (9A) Includes a vertical bar graph showing the percent inhibition of α-Synuclein-induced TNFα secretion exerted by the various compounds and combinations. As shown, neither CLX (at concentrations of 1, 2.5, or 5 µM) nor CBD (at concentrations of 1, 2.5 and 5 µM) by themselves produced significant inhibitory effects. A combination of CLX 1 µM and CBD 5 µM produced a significantly greater inhibition than CLX by itself. A combination of CLX 2.5 µM plus CBD 5 µM produced a significant inhibitory effect. All combinations of CLX at 5 or 10 µM plus each concentration of CBD produced significant inhibitory effects. *p<0.05 compared with α-Synuclein -exposed cultures treated with Veh. ^{#}p<0.05 compared with cultured treated with the corresponding concentration of CLX by itself. (9B) Includes a concentration-response plot of the effects of the various compounds and combinations. Based on this plot, the IC50 of CLX and CBD, by themselves or in combination, was computed. The IC50, i.e., the concentration of CLX and CBD, by themselves or in combination, where the response to LPS is reduced by half of the maximal effect, is presented in the Table on the right. As shown, the IC50 of CLX by itself was markedly reduced (i.e., the effect of CLX was potentiated) when CLX was added together with each concentration of CBD.

FIGS. 10A-10D include vertical bar graphs describing the effects of cannabinoids, terpenes, and flavonoids, as well as some of their combinations, on depressive-like symptoms in LPS-treated mice. FIG. 10A is a vertical bar graph showing the effects of the various compounds and combinations on the LPS-induced decrease in the distance moved in the open field. The findings reflect the significant attenuation of the motivational, psychomotor and anxiogenic aberrations associated with the inflammatory condition, by treatment with CBD (30 mg/kg), CBDA (at both 1 and 5 mg/kg), CBD plus THC (30 and 10 mg/kg, respectively), and CBD plus β-caryophyllene (BCP, 30 and 50 mg/kg, respectively). FIG. 10B is a vertical bar graph showing the effects of the various compounds and combinations on the LPS-induced decrease in sucrose preference. The findings reflect the significant attenuation of the anhedonia associated with the inflammatory condition, by treatment with CBD (30 mg/kg), CBDA (at both 1 and 5 mg/kg), BCP (50 mg/kg), kaempferol (30 mg/kg), CBD plus THC (10 and 10 mg/kg, respectively), CBD plus THC (10 and 30 mg/kg, respectively), and CBD plus kaempferol (30 and 30 mg/kg, respectively). FIG. 10C is a vertical bar graph showing the effects of the various compounds and combinations on the LPS-induced decrease in Social Exploration. The findings reflect the significant attenuation of the motivational and social aberrations associated with the inflammatory condition, by treatment with CBD (30 mg/kg), linalool (50 mg/kg), and CBD + β-caryophyllene (BCP, 30 and 50 mg/kg respectively). FIG. 10D is a vertical bar graph showing the effects of the various compounds and combinations on LPS-induced decrease in food consumption. The findings reflect the significant attenuation of the motivational and social aberrations associated with the inflammatory condition, by treatment with CBD (30 mg/kg), and CBD + THC (30 and 10 mg/kg, respectively), whereas cannabinol (10 mg/kg) and CBD (30 mg/kg) plus BCP (50 mg/kg) significantly reduced food consumption. *p<0.05 compared with the Vehicle-LPS treated group.

FIGS. 11A-11L include vertical bar graphs describing the effects of CBD on LPS-induced depressive- and anxiety-like symptoms in adult (6-9 months-old) and young adult (2.5-3 months old) male mice. In adult mice, LPS administration (300 µg/kg) significantly reduced the distance travelled by mice in the open field arena, compared with saline-injected controls, indicating LPS-induced decrease in exploratory/anxiety-related behavior. CBD treatment attenuated this effect of LPS, i.e., the distance moved by LPS-injected mice treated with CBD was significantly increased when compared to LPS-injected mice treated with vehicle (11A). Sucrose preference (11B) and sucrose consumption (11C) were significantly reduced in adult mice that were injected with LPS, when compared to saline-injected mice. These LPS-associated anhedonic effect was almost completely reversed in CBD-treated mice. LPS caused a deficit in social behavior, as demonstrated by reduced amount of time that LPS-injected mice spent exploring a juvenile conspecific in the social exploration test. LPS-injected mice that were treated with CBD showed significantly increased social interest when compared to LPS-injected vehicle-treated adult mice (11D). LPS caused body weight loss (11E) and a decrease in food consumption (anorexia) (11F), which were not influenced by CBD administration. In young-adult mice, LPS induced decreases in the distance moved in the open field test (11G), in sucrose preference (11H) and sucrose consumption (11I), in body weight (11K) and in food consumption (11L). None of these parameters was influenced by treatment with CBD. LPS had no effect of social exploration (11J). *p<0.05 compared with the corresponding Sal-injected group. # p<0.05 compared with the Vehicle-treated LPS-injected group.

FIGS. 12A-12F include vertical bar graphs describing the behavioral and microglial effects of 3 daily consecutive injections of CBD in young-adult mice, with the last injection preceding by 1 hr the injection of LPS (or saline control). LPS induced significant decreases in the distance moved in the open field test, sucrose preference, sucrose consumption, social exploration, and body weight (12A-12D, respectively). The repeated (prophylactic) CBD administration attenuated the effects of LPS on the distance travelled in the open field test, but this effect did not reach statistical significance (12A). In the sucrose preference test, CBD significantly reversed the LPS-induced reduction in preference (12B), but it had no effect on LPS-induced reduction in sucrose consumption (12C). CBD had no effects on LPS-induced reductions in social exploration (12D) and body-weight loss (12E). CBD caused a significant reduction in hippocampal microglia numbers, compared to Veh-LPS controls, and compared to Veh-Sal controls (12F). Together, these findings indicate that in contrast with the lack of an acute effect of CBD in young-adult mice, semi-chronic (prophylactic) CBD administration can reverse the effects of LPS on anhedonia, in association with a reduction in microglia density. *p<0.05 compared with the Sal-injected group. # p<0.05 compared with the Vehicle-treated LPS-injected group.

FIGS. 13A-13B include vertical bar graphs describing the effects of oral treatment with a CBD-containing Pro NanoLipospheres (PNL) formulation on LPS-induced depressive/anxiety-like symptoms. Mice consumed the formulation for 4 days. For the low and high CBD-treated groups, the dose/day (47 and 78 mg/kg, respectively) was computed based on the CBD concentration in the PNL solution and the consumption values of each group. (13A) Mice treated with either PNL solution without CBD or with CBD solution at 47 mg/kg showed a significant LPS-induced reduction in the distance moved in the open field, compared with saline-injected mice. In contrast, no such difference was found with respect to mice treated with the 78 mg/kg solution. (13B) Mice treated with PNL solution by itself or with 47.1 mg/kg CBD displayed a significant reduction in sucrose preference, compared with both the saline-treated mice and mice treated with 78 mg/kg CBD. *p<0.05 compared with the Sal-injected group. **p<0.01 compared with the Sal-injected group. ^{#}p<0.05 compared with the 78 mg/kg CBD-injected group. ^{##}p<0.01 compared with the 78 mg/kg CBD-injected group.

FIGS. 14A-14L include vertical bar graphs describing the effects of CBD on LPS-induced depressive- and anxiety-like symptoms in adult (6-9 months-old) and young adult (2.5-3 months old) female mice. In both age groups, LPS administration significantly reduced the distance travelled by mice in the open field test (14A, and 14G), sucrose preference (14B, and 14H), sucrose consumption (14C, and 14I), social exploration (14D, and 14J), food consumption (14E, and 14 K) and body weight (14F, and 14L), compared with saline-injected controls. None of these parameters was influenced by treatment with CBD, in either saline or LPS-treated mice. *p<0.05 compared with the corresponding Sal-injected group. ^{#}p<0.05 compared with the Vehicle-treated LPS-injected group.

FIGS. 15A-15E include vertical bar graphs describing the effects of CBD, kaempferol and their combinations on LPS-induced depressive/anxiety-like symptoms in adult female mice. Administration of kaempferol (60 mg/kg) by itself or in combination with CBD (i.e., at a 30:60 mg/kg ratio), but not the combination of CBD and kaempferol at the 30:15 ratio or CBD by itself, significantly mitigated the negative effect that LPS had on the distance travelled in the open field test (15A). Administration of the combination of CBD and kaempferol at the 30:15 mg/kg ratio, but not at the 30:60 ratio or any of the compounds separately, significantly attenuated LPS-induced suppression of sucrose preference (15B). However, only the combination of CBD and kaempferol at the 30:15 mg/kg ratio attenuated the LPS-induced reduction in sucrose consumption measurement (15C). LPS-induced body-weight loss was attenuated only by the CBD and kaempferol at the 30:15 mg/kg ratio, but not at the 30:60 ratio (15D), whereas LPS-induced suppression of food consumption was attenuated only by the combination of CBD and kaempferol at the 30:15 mg/kg ratio (15E). *p<0.05 compared with the Vehicle-treated Saline-injected group. # p<0.05 compared with the Vehicle-treated LPS-injected group. & p<0.05 compared with the LPS-injected CBD-treated group. % p<0.05 compared with the LPS-injected kaempferol-treated group. $p<0.05 compared with the LPS-injected CBD:kaempferol 30:15 combination-treated group.

FIGS. 16A-16K include vertical bar graphs describing the effects of cannabidiolic acid (CBDA) on depressive/anxiety-like symptoms in LPS-injected adult female and male mice. In female mice, CBDA at 1, but not 0.5 or 5 mg/kg, significantly increased sucrose preference (16A), and sucrose consumption (16B) as compared with Veh-treated LPS-injected mice. CBDA at 5, but not 0.5 or 1 mg/kg significantly increased the distance travelled in the open field test, as compared with Veh-treated LPS-injected mice (16C). While none of the CBDA doses attenuated the body-wight loss (16D), CBDA at 1 mg/kg significantly elevated food consumption (16E) as compared with Veh-treated LPS-injected mice. In male mice, CBDA at 1, but not 5 mg/kg or in combination with CBD, significantly increased sucrose preference, as compared with Veh-treated LPS-injected mice (16F). Sucrose consumption was significantly increased by CBDA, at both 1 and 5 mg/kg, as well as by the combination of CBDA and CBD (albeit not to a greater extent than CBDA by itself) (16G). In the forced swim test, CBDA had no effect on immobility time (16H), and CBDA at 5, but not 0.5 or 1 mg/kg or in combination with CBD significantly increased the latency to first floating, as compared with Veh-treated LPS-injected mice (16I). Neither CBDA nor the combination of CBDA and CBD had an effect on body-weight loss (16J) and food consumption (16K). *p<0.05 compared with the Veh-treated LPS-injected group. ^{#}p<0.05 compared with the group treated with the combination of CBD and CBDA.

FIGS. 17A-17H include vertical bar graphs describing the effects of cannabidiolic acid (CBDA) on hippocampal microglia density and morphology in LPS-injected mice. In female mice, CBDA at 1, but not 0.5 or 5 mg/kg, significantly reduced microglia density (17A). CBDA had no effects on microglial soma area (17B) or processes length (17C), but administration of CBDA at 5 mg/kg significantly elevated the number of microglia branch points (17D). In male mice, CBDA at 1, but not 5 mg/kg or in combination with CBD, significantly reduced microglia density, compared with Veh-treated LPS-injected mice (17E). CBDA had no effects on microglial soma area (17F) or processes length (17G), but administration of CBDA at 1 mg/kg significantly elevated the number of microglia branch points (17H). *p<0.05 compared with the Veh-treated LPS-injected group.

FIGS. 18A-18D include vertical bar graphs depicting the effects of CLX (10 mg/kg), CBD (5 mg/kg), the combination of the two drugs and vehicle control, on the activity of mice in the Forced Swim Test and the Elevated Plus Maze Test. (18A) The combination of CLX plus CBD significantly reduced the immobility time (in seconds (sec), out of a total of 360 sec), when compared with mice that were injected with vehicle. There were no significant effects of sex, treatment by sex interaction or treatments with each compound by itself. (18B) The combination of CLX plus CBD significantly increased the latency to the first episode of immobility, compared with the Vehicle-treated group, as well as compared with the groups treated by CLX and CBD, each by itself. (18C) In the Elevated Plus Maze, males were found to spend significantly more time in the open arms than females. Although there was no overall treatment effect, comparison of the effect of the CLX plus CBD combination group vs. the Vehicle treated group showed a clear trend for a differential effect (p=0.057). (18D) Similar effects were obtained with respect to the entries into the open arms, however the findings did not reach statistical significance. These findings suggest that when combined with CLX, CBD can produce a dose-sparing effect and potentiate the antidepressant effect of CLX. CBD may also similarly influence the anxiolytic effect of CLX, although this finding needs further corroboration. *p<0.05 compared with the Vehicle-treated group. #p<0.05 compared with the CLX-treated group. & p<0.05 compared with CBD-treated group.

FIGS. 19A-19D include vertical bar graph depicting the effects of CBD (30 mg/kg) and three doses of CLX (10, 20 or 30 mg/kg), either by themselves or in various combinations of CBD and CLX (at 30:10, 30:20 and 30:30 ratios) on LPS-induced anxiety and depressive-like behaviors. (19A) In the open field test, the LPS-injected vehicle (Veh)-treated group displayed a significant reduction in the distance moved, compared with the saline (Sal)-injected group. Treatment with CLX (30 mg/kg) by itself produced a significant increase in the distance travelled compared with the LPS-injected Veh-treated group, whereas administration of 10 or 20 mg/kg CLX had no such effect. Combinations of CLX plus CBD, at either 20:30 or 30:30 ratios, significantly increased the distance travelled compared with the vehicle-LPS group, as well as the CBD only group. Finally, the combination of CLX plus CBD at the 20:30 ratio, was also significantly different from the CLX at the corresponding dose (20 mg/kg), reflecting a potentiated, dose-sparing effect. (19B-19C) Assessment of the drug effects on the distance moved and the number of entries into the center of the open-field (an established measure of anxiolytic effects) revealed no significant effects of CLX or CBD by themselves, except for a significant increase in the number of entries following 30 mg/kg CLX (all compared with the vehicle-LPS group). Combinations of CLX plus CBD, at all ratios for the distance in center and entries produced a significant elevation compared with the vehicle-LPS group. For the distance in center the combinations of 30:30 was different from the CBD group, and for the number of entries both the 10:30 and 30:30 combinations were different from the CBD group. Finally, the number of entries following treatment with the combination of CLX plus CBD at 10:30 ratio was significantly higher than the number in the CLX 10 mg/kg by itself. (19D) In the Sucrose Preference test, there were no significant effects of CLX or CBD by themselves, whereas the combinations of CLX and CBD, at 20:30 and 30:30 ratios, produced a significant increase in sucrose preference, compared with the vehicle-LPS group. The sucrose preference following treatment with the combination of CLX plus CBD at the 10:30 ratio was higher than the preference of the group treated with 10 mg/kg of CLX by itself. *p<0.05 compared with the Vehicle-treated Saline-injected group. # p<0.05 compared with the Vehicle-treated LPS-injected group. & p<0.05 compared with the LPS-injected CBD-treated group. % p<0.05 compared with the respective LPS-injected CLX-treated group.

FIGS. 20A-20F include a vertical bar graph depicting the effects of CLX, CBD and a combination of CBD plus CLX (each at 30 mg/kg) on the depressive- and anxiety-like effects induced by chronic social defeat stress (CSDS). (20A-20B) In the Open Field Test, the vehicle-treated group displayed a significant reduction in the total distance moved and the distance moved in the center, compared with the naïve group; this reduction was partially (but significantly) reversed by treatment with the CLX plus CBD combination. (20C-20D) In the EPM test, the Vehicle-treated group displayed significantly lower ratio of open arms entries compared to the naïve group, and this effect was completely rescued by the CLX plus CBD combination. A similar result was obtained with the ratio of open arms time (i.e., the time spent in the open arms divided by the time spent in both the open and the closed arms), but the results for this measure did not reach statistical significance. (20E) In the social Exploration (SE) test, mice in the vehicle treatment group displayed a small reduction in SE, as compared with the naïve group mice, which did not reach statistical significance. However, SE levels in the CLX plus CBD group were significantly higher than in the vehicle-treated group, suggesting a pro-social effect of the drug treatment. (FIG. 20F) In the sucrose preference test, the vehicle-treated group displayed significantly lower sucrose preference compared to the naïve group. This effect was completely rescued by the CLX plus CBD combination. Together, these findings demonstrate that the CLX plus CBD combination induces anxiolytic, pro-social and antidepressant effects in the CSDS model. *p<0.05 compared with the naïve group. #p<0.05 compared with the vehicle-treated stress group.

FIG. 21 includes an illustration scheme of a non-limiting scheme depicting that microglia-suppressive formulation of the present invention, exerts antidepressant and anxiolytic effects in patients with high baseline levels of MINDD (Markers of Inflammation in Neuropsychiatric Diseases), including CRP and/or CCL11 and/or IL-6, and/or TNFα, and/or IL-8, and/or MIP-1α, and/or MCP1, but not in patients with low levels of these biomarkers. In a patient with high CRP and/or other MINDD levels, the formulation reduces scores on the Montgomery-Åsberg Depression Rating Scale (MADRS), particularly the vegetative symptom factor of this scale, as well as the Hamilton Anxiety Rating Scale (HARS), the Clinical Global Improvement and Severity Scales (CGI-S and CGI-I), the Snaith-Hamilton Pleasure Scale (SHAPS), and depression-associated somatic symptoms, assessed by the Somatic Symptoms Scale -2 (SSS-8). In patients with either high or low baseline MINDD levels, placebo produces only mild and similar effects on the measures of depression (MADRS scores), vegetative symptoms, somatic symptoms, anxiety, and global improvement.

FIG. 22 includes an illustration scheme of a non-limiting scheme depicting that microglia-suppressive formulation of relatively low doses of CLX plus CBD, or a formulation of whole plant cannabis extract in combination with a relatively low dose of CLX, exerts antidepressant and anxiolytic effects in patients with high, but not low baseline levels of MINDD (Markers of Inflammation in Neuropsychiatric Diseases), including CRP and/or CCL11 and/or IL-6, and/or TNFα, and/or IL-8, and/or MIP-1α, and/or MCP1. A similar treatment with placebo or CLX or CBD by themselves, produces significantly smaller effects in depressed patients with either high or low baseline MINDD levels (i.e., the addition of CBD to CLX enables a dose-sparing effect of the latter). Specifically, in a patient with high levels of CRP and/or other MINDD, the formulation of CLX plus CBD reduces scores on the Montgomery-Åsberg Depression Rating Scale (MADRS), particularly the vegetative symptom factor of this scale, as well as the Hamilton Anxiety Rating Scale (HARS), the Clinical Global Improvement and Severity Scales (CGI-S and CGI-I), the Snaith-Hamilton Pleasure Scale (SHAPS), and depression-associated somatic symptoms, assessed by the Somatic Symptoms Scale -2 (SSS-8). In patients with either high or low baseline MINDD levels, placebo or CLX or CBD by themselves, produce only mild effects on these psychiatric measures.

FIG. 23 includes a scheme of a non-limiting scheme depicting proof of concept results validating the personalized therapeutic approach for the diffuse/malignant (DM) subtype of Parkinson’s disease (PD)-associated emotional and cognitive disturbances, based on microglia-suppressive formulation of the present invention. Patients with the diffuse/malignant (DM) subtype of PD are selected based on the following characteristics: depression (MADRS score >17), cognitive impairment (MoCA score≤26), orthostatic hypotension, rapid eye movement sleep behavior disorder (RBD Questionnaire score ≥ 5 points), excessive daytime sleepiness (ESS score > 8), and fast progression of the disease. The inflammatory status of the patients is determined by examination of baseline plasma levels of the inflammatory markers CRP, and/or IL-1β, and/or IL-6, and/or IL-8, and/or TNF-α, and/or MCP-1, and/or MIP-1α, and/or MIP-1β, and and/or RANTES. Treatment with microglia-suppressive formulation of cannabinoid(s) or whole plant cannabis extract, by itself, or with terpene(s) and/or flavonoid(s) and/or CLX in patients with the DM subtypes who have high (but not those who have low) baseline levels of said inflammatory markers, exhibit significant improvements in: 1) emotional functioning (particularly depression, assessed by the Montgomery-Asberg Depression Rating Scale (MADRS)), and anxiety (assessed by the HARS). 2) cognitive functionating (assessed by the Montreal Cognitive Assessment (MoCA)), 3) health status and quality of life (assessed by the PDQ-39 questionnaire), 4) autonomic functioning (assessed by the Scales for Outcomes in Parkinson’s Disease — Autonomic (SCOPA-AUT)), 5) behavioral parameters (particularly sleep behavior and quality, assessed by the REM Sleep Behavior Disorder Screening Questionnaire, the Epworth Sleepiness Scale (ESS) and the Pittsburgh Sleep Quality Index (PSQI)), and 6) global symptom severity and their improvement (assessed by the Clinical Global Impression — Severity scale (CGI-S) and Improvement scale (CGI-I)). When treated with vehicle, such patients display either no or only minor changes in their emotional, cognitive and general health symptoms, regardless of their baseline inflammatory state.

FIG. 24 includes an illustration scheme of a non-limiting scheme depicting that microglia-suppressive formulation of the present invention exerts antidepressant effects in depressed Alzheimer’s disease patients with high baseline levels of MINDD (Markers of Inflammation in Neuropsychiatric Diseases), including CRP and/or CCL11 and/or IL-6, and/or TNFα, and/or IL-8, and/or MIP-1α, and/or MCP1, but not in patients with low MINDD levels. In an AD patient with depression (defined as a score above 8 in the Cornell Scale for Depression in Dementia (CSDD)) who has a high MINDD levels, the formulation significantly reduces the CSDD scores by more than 4 points (primary objective). Since depression exacerbates the cognitive impairments, these patients also exhibit significant improvements in the Alzheimer’s Disease Assessment Scale (ADAS-cog), the Clinician’s Interview Based Impression of Change with caregiver input (CIBIC+), the MMSE, Clinical Dementia Rating (CDR), and the AD Cooperative Study Activities of Daily Living scale (ADCS-ADL) (secondary objectives). In patients with either high or low baseline MINDD levels, placebo produces only mild and similar effects on the measures of depression (CSDD scores), and no effects on the cognitive functioning scores.

FIGS. 25A-25F depict the effects of several CBD-rich and THC-rich Cannabis extracts, as compared with plant-derived CBD, on LPS-induced secretion of pro-inflammatory cytokines by BV2 microglia cell cultures. (25A-25C) are vertical bar graphs showing the percent inhibition of LPS-induced TNFα, IL-6 and IL-1β protein levels exerted by CBD alone or by several Cannabis whole-plant extracts (CannA, CannB and CannC). (25A) As shown, the CannA extract significantly inhibited TNFα protein release from activated microglia at concentrations of 5 and 10 µM, whereas CBD and CannB inhibited TNFα release only at the high concentration of 10 µM. The CannC extract did not influence TNFα. (25B) Significant inhibition of IL-6 protein release from activated microglia was exerted by CBD, CannA and CannB at 5 and 10 µM, whereas CannC produced significant IL-6 inhibitory effects only at 10 µM. (25C) Concentrations as low as 2.5 µM, as well as 5 and 10 µM of CannA and CannB extracts significantly lowered the release of IL-1β from activated microglial cells, while CBD and CannC extract were effective only at 5 µM and 10 µM (25C). *p<0.05, **p<0.01, ***p<0.001 as compared to LPS-exposed cultures treated with vehicle (Veh). FIGS. 25D-F are concentration-response plots, showing nonlinear regression curves fitted based on wide-range concentration-response experiments of the TNFα (25D), IL-6 (25E) and IL-1β (25F) suppressive effects of purified CBD and of the various Cannabis extracts. Based on these plots, the IC50 values were computed, defined as the concentrations of CBD or the Cannabis extract (calculated based the known concentration of the dominant cannabinoid, i.e., CBD in the CannA and CannB extracts or THC in the CannC extract), at which the response to LPS is reduced by half of the maximal effect. The Table on the right of each XY plot demonstrates the IC₅₀ concentrations for each treatment.

### DETAILED DESCRIPTION

The present invention relates to methods and compositions for treating a mood disorder in a subject in need thereof.

### Method of Treatment

In some embodiments, there is provided a method for treating or attenuating a mood disorder in a subject in need thereof, the method comprising: (a) determining whether the subject has a high inflammatory state and/or microglial activation state; and (b) administering to the subject determined as having a high inflammatory state and/or microglial activation state a pharmaceutical composition comprising a therapeutically effective amount of microglia suppressive formulation comprising a first cannabinoid and at least one additional therapeutic compound.

In some embodiments, the at least one additional therapeutic compound is an anti-inflammatory drug, an anti-microglial drug, or a combination thereof (e.g., an NSAID). In some embodiments, the at least one additional therapeutic compound is selected from: at least one cannabinoid being different from the first cannabinoid, a terpene, and a flavonoid.

In some embodiments, the method comprises administering of a pharmaceutical composition comprising a cannabinoid, a terpene, and a flavonoid.

In some embodiments, the first cannabinoid, the at least one additional therapeutic compound, or both, are synthetic.

In some embodiments, the first cannabinoid, the at least one additional therapeutic compound, or both are isolated, extracted, or purified from a plant or a plant part.

In some embodiments, the first cannabinoid is synthetic and the at least one additional therapeutic compound is isolated, extracted, or purified from a plant or a plant part.

In some embodiments, the first cannabinoid is isolated, extracted, or purified from a plant or a plant part and the at least one additional therapeutic compound is synthetic.

In some embodiments, the first cannabinoid and the at least one additional therapeutic compound are obtained or derived, e.g., isolated, extracted, or purified, from distinct sources. In some embodiments, the first cannabinoid and the at least one additional therapeutic compound are not obtained or derived from the same source.

As used herein, a plant part is selected from: a seed, a leaf, a root, a shoot, a stem, a flower, a fruit, a stolon, a bulb, a tuber, a corm, a bud, trichomes, or any combination thereof.

In some embodiments, a plant as used herein refers to a whole plant.

In some embodiments, a compound isolated, extracted, or purified from a plant is in a composition comprising a whole plant extract, a plant part extract, or any fraction thereof.

In some embodiments, there is provided a method for treating or attenuating a mood disorder in a subject having a high inflammatory state and/or microglial activation state, the method comprising administering to the subject a pharmaceutical composition comprising a therapeutically effective amount of microglia suppressive formulation comprising at least one cannabinoid, at least one terpene, and/or at least one flavonoid.

In some embodiments, the subject has a high inflammatory state and/or microglial activation state.

As used herein, “ a high inflammatory state and/or microglial activation state” is compared to control. In some embodiments, a control is a healthy subject. In some embodiments, a control is a subject not being afflicted with inflammation. In some embodiments, a control is a subject not being afflicted with a mood- or stress-related disease or disorder. In some embodiments, a subject having or comprising high inflammatory/microglia state is characterized by having any one of: (a) plasma C-reactive protein (CRP) levels greater than 1 mg/L; (b) plasma eotaxin-1 (CCL11) levels greater than 72 pg/ml; (c) plasma Interleukin (IL)-6 levels greater than 2.0 pg/ml; (d) plasma Tumor necrosis factor (TNF)-α levels greater than 3.8 pg/ml; (e) plasma IL-8 levels greater than 31 pg/ml, (f) plasma macrophage inflammatory protein (MIP)-1α (also termed CCL3) levels greater than 7.1 pg/ml, (g) plasma monocyte chemoattractant protein (MCP)-1 (also termed CCL2) levels greater than 80.0 pg/ml, and any combination thereof.

In some embodiments, the method comprises a step of detecting a high inflammatory/microglial state of in subject. In some embodiments, detecting comprises determining the plasma level of at least one inflammatory marker selected from CRP, CCL11, IL-6, TNFα, MIP-1α, and MCP1, wherein a level of any one of: (i) more than 1 mg/L CRP, (ii) more than 72 pg/ml CCL 11 (eotaxin-1), (iii) more than 2.0 pg/ml IL-6, (iv) more than 3.8 pg/ml TNFα, (v) more than 31 pg/ml IL-8, (vi) more than 7.1 pg/ml MIP-1α, (vii) more than 80 pg/ml MCP-1, and any combination thereof, is indicative of the subject has an inflammatory/microglia state suitable for treatment using the microglia suppressive formulation.

In some embodiments, the high inflammatory state and/or microglia activation state is determined by a dedicated assay kit, termed MINDD (Markers of Inflammation in Neuropsychiatric Diseases), measuring the levels of plasma CRP, IL-1β, IL-6, TNFα, CCL11, IL-8, MIP-1α, MCP1, RANTES, or any combination thereof.

In some embodiments, the method comprises determining whether the subject has a high inflammatory/microglial state in a sample derived from the subject. In some embodiments, the method further comprises providing a sample from the subject and performing the determining in the sample.

As used herein, the term sample refers to any type of physical specimen which has been obtained, collected, derived, dissected or any equivalent thereof, from subject. In some embodiments, the sample comprises biological fluids selected from: serum, plasma, vitreous fluid, lymph fluid, synovial fluid, follicular fluid, seminal fluid, amniotic fluid, milk, whole blood, urine, cerebrospinal fluid, saliva, sputum, tears, perspiration, mucus, or tissue culture media, or a combination thereof. Each possibility represents a separate embodiment of the invention. In some embodiments, the sample is selected from: tissue extracts, homogenized tissue, cellular extracts, or a biopsy, or a combination thereof. Each possibility represents a separate embodiment of the invention.

In some embodiments, a high inflammatory state and/or microglial activation state comprises microglia activation.

As used herein, “microglia activation” refers to any physical or biochemical change skewing the microglia from homeostatic state, and includes but is not limited to, rapid proliferation, migration to the site of pathology, phagocytosis of a cell and/or debris, and production and/or secretion of the cytokines and chemokines necessary to stimulate microglia and/or other brain and/or immune cells. “Microglia activation” further refers to cases when infection, injury or disease occur in the brain and affect nerve cells, microglia in the central nervous system become “active,” causing inflammation in the brain, similar to the manner in which white blood cells act in the rest of the body. In another embodiment, microglia act like the monocyte phagocytic system. In some embodiments, activated microglia can generate large quantities of superoxide anions, with hydroxyl radicals, singlet oxygen species and hydrogen peroxide being a downstream product, any of which can be assayed in the preparations utilized in such methods of the invention.

Active microglia may be characterized by at least one of the following characteristics: (1) their cell bodies becoming larger, their processes becoming shorter and thicker, (2) an increase in the staining for several molecular activation markers, including Iba-1 and MHC-II (3) proliferation and clustering, (4) production and secretion of inflammatory mediators, including pro-inflammatory (e.g., interleukin (IL)-1, IL-6, nuclear factor kappa-light-chain-enhancer of activated B cells (NF-κB) and tumor necrosis factor-α) and anti-inflammatory (e.g., IL-10, IL-1ra) cytokines, inflammatory chemokines (e.g., IL-8, MIP-1α, MCP1), as well as additional inflammatory mediators (e.g., prostaglandins), (5) production and secretion of various neuroprotective factors, including brain-derived neurotrophic factor (BDNF) and insulin growth factor-1 (IGF-1), (6) production and secretion of chemo-attractive factors (chemokines), which recruit microglia from within the brain to specific brain locations and facilitate the infiltration of peripheral immune cells, for example, white blood cells, as compared to that found in the non-reactive state, and (7) low levels of microglia inhibitory checkpoints, including CX3CR1, CD200 and LAG-3. In some embodiments microglial activation is determined in at least one brain region or area, such as in the hippocampal dentate gyrus (DG), in the prelimbic cortex or in any depression-related area. In some embodiments, an activated microglia cell and not a resting microglia cell promotes inflammation by expressing inhibitor of nuclear factor kappa-B kinase subunit beta (IKKB). In one embodiment, an activated microglia cell comprises an activated EGF-like module-containing mucin-like hormone receptor-like 1 (EMR1) promoter. In one embodiment, an activated microglia cell comprises an activated Chemokine CX3C motif receptor 1 (CX3CR1) promoter. In one embodiment, an activated microglia cell comprises an activated integrin alpha M (ITGAM) promoter. In one embodiment, an activated microglia cell comprises an activated toll-like receptor 4 (TLR4) promoter. In one embodiment, an activated microglia cell comprises an activated cluster determinant 14 (CD14) promoter. In one embodiment, an activated microglia cell comprises a suppressed lymphocyte-activating gene-3 (LAG-3) checkpoint receptor promoter.

In some embodiments, the method comprises a step of determining microglia activation using positron emission tomography (PET).

In some embodiments, PET analysis, for example translocator protein-18 kDa (TSPO) PET is used so as to determine the binding level of a labeled ligand.

In some embodiments, a binding level of at least 40% more, at least 50% more, at least 60% more, at least 70% more, at least 80% more, or at least 90% more than the binding level in an age-matched healthy control, or any value and range therebetween, is indicative of the subject having activated microglia or undergoing microglia activation. Each possibility represents a separate embodiment of the invention.

In some embodiments, a binding level of 40-90% more, 45-95% more, 50-85% more, 60-90% more, 55-75% more, and 65-90% more than the binding level in an age-matched healthy control, is indicative of the subject having activated microglia or undergoing microglia activation. Each possibility represents a separate embodiment of the invention.

In some embodiments, a subject having activated microglia or undergoing microglia activation is afflicted with an inflammatory/microglial state suitable for treatment according to the method of the invention.

In some embodiments, a subject having activated microglia or undergoing microglia activation is afflicted with a high inflammatory state and/or microglial activation state suitable for treatment using a pharmaceutical composition disclosed hereinbelow.

PET analysis and types of ligands suitable for determining microglia activation are common and would be apparent to one of ordinary skill in the art. Non-limiting examples of TSPO binding ligands include, but are not limited to [¹¹C] (R)PK11195, [¹⁸F]-FEPPA, [¹¹C]PBR28, [¹⁸F]DPA, and others.

In some embodiments, the subject is afflicted with a condition selected from: depression, vegetative state of depression, high body mass index (BMI), atypical depression, high state anxiety, high number of previous depressive episodes, childhood adversity, and feeling unloved as a child or wishing for a different childhood. In some embodiments, a subject afflicted with any one of the hereinabove conditions is resistant to standard medication used for treating the condition. In some embodiments, resistant is unresponsive. In some embodiments, resistant refers to the that the medication reliefs one or more symptoms of the condition but not all. In some embodiments, resistant refers to that the medication lowers the severity of one or more symptoms of the condition but not all. In some embodiments, resistant refers to that the medication does not fully treat, cure, or heal the subject. In some embodiments, resistant refers to that the condition is refractory. The terms “resistant” and “resistance” are used herein interchangeably.

In some embodiments, a mood disorder is selected from: major depressive disorder, unipolar major depressive episode, dysthymic disorder, treatment-resistant depression, bipolar depression, adjustment disorder with depressive mood, depression associated with REM sleep behavior disorder, cyclothymic disorder, atypical depression, seasonal affective disorder, depression co-morbid with anxiety disorder, generalized anxiety disorder, melancholic depression, psychotic depression, post-schizophrenic depression, schizophrenia, depression due to a general medical condition, depression associated with a synucleinopathy (including Parkinson’s disease, particularly with the diffuse/malignant subtype, or dementia with Lewy bodies (DLB), or multiple system atrophy (MSA), depression associated with autosomal-recessive Parkinson’s disease, depression associated with Alzheimer’s disease, depression associated with multiple sclerosis, depression associated with Tourette’s disorder, depression associated with dystonia, depression associated with ataxia, depression associated with dyskinesia, depression associated with essential tremor, depression associated with PTSD, post-viral fatigue syndrome, chronic fatigue syndrome, depression associated with autism spectrum disorder, depression associated with schizophrenia, depression associated with somatoform disorder, depression associated with somatic symptom disorder, depression associated with pain disorder, depression associated with rheumatoid arthritis, depression associated with osteoarthritis, depression associated with ankylosing spondylitis, depression associated with lupus erythematosus, depression associated with Crohn’s disease, depression associated with inflammatory bowel disease, depression associated with Williams syndrome, depression associated with the DiGeorge syndrome, depression associated with cancer, depression associated with primary biliary cholangitis, depression associated with autoimmune hepatitis, depression associated with neurofibromatosis, and fibromyalgia.

In some embodiments, any sleepiness or sleep disturbance as described herein, is induced by or is associated with depression.

In some embodiments, sleepiness or sleep disturbance as described herein, is a condition or a symptom associated with or induced by depression.

In some embodiments, the method of the invention is directed to treating a mood disorder due to a general medical condition in a subject, wherein the mood disorder is promoted, propagated, induced, initiated, or any equivalent thereof, due to a general medical condition in the subject.

As used herein, the term “mood disorder due to a general medical condition” refers to a manic or depressive episode which occurs secondary to a medical condition. Non-limiting examples of medical conditions which may trigger a mood episode include, but are not limited to, a neurological disorder (e.g., dementias), a metabolic disorder (e.g., electrolyte disturbances), a gastrointestinal disease (e.g., cirrhosis), an endocrine disease (e.g., thyroid abnormalities), a cardiovascular disease (e.g., heart attack), a pulmonary disease (e.g., chronic obstructive pulmonary disease), cancer, and an autoimmune diseases.

In some embodiments, the method of the invention is directed to treating a specific domain of major depression. In some embodiments, the method of the invention is directed to treating anhedonia. In some embodiments, the method of the invention is directed to treating vegetative symptoms of depression, including one or more of the following symptoms: weight loss and anorexia (loss of appetite), fatigue, and low energy. In some embodiments, the method of the invention is directed to treating somatic symptoms of depression, including painful physical symptoms, such as headache, backache, stomachache, and joint and muscle aches.

In some embodiments, the method further comprises an anti-inflammatory procedure or an anti-microglial procedure.

In some embodiments, the anti-inflammatory procedure or the anti-microglial procedure comprises a non-invasive brain stimulation (NIBS). In some embodiments, the NIBS is selected from the group consisting of: electroconvulsive therapy (ECT), repetitive transcranial magnetic stimulation (rTMS), deep TMS, cranial electrotherapy stimulation (CES), transcranial direct current stimulation (tDCS), transcranial random noise stimulation (tRNS), and reduced impedance non-invasive cortical electrostimulation (RINCE).

In some embodiments, alleviation or treatment of neurovegetative symptoms of depression and/or depression-associated somatic symptoms, are measured by the change in the vegetative factor of the Montgomery-Åsberg Depression Rating Scale (MADRS), the Somatic Symptom Scale-8 (SSS-8), or both.

In some embodiments, alleviation or treatment are measured by the change in the Patient Health Questionnaire (PHQ-15), the Hamilton Anxiety Rating Scale (HARS), the Clinical Global Improvement and Severity Scales (CGI-S), or any combination thereof.

In some embodiments, the method further comprises a step of determining the change in the MADRS scale, the SSS-8 scale, PHQ-15, HARS, CGI-S, or any combination thereof.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject’s quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compositions or formulations prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

In some embodiments, the subject is a mammal. In some embodiments, the subject is a human subject.

### Composition

According to some embodiments, there is provided a pharmaceutical composition comprising a first cannabinoid, at least one additional therapeutic compound, and a pharmaceutically acceptable carrier.

In some embodiments, the at least on additional therapeutic compound is selected from: at least one cannabinoid being different from the first cannabinoid, a terpene, and a flavonoid.

In some embodiments, the herein disclosed composition is for use in treating a mood disorder. In some embodiments, the herein disclosed composition is a microglia suppressive formulation.

In some embodiments, the first cannabinoid and the at least one cannabinoid being different from the first cannabinoid are selected from: cannabidiol (CBD), Δ-9-tetrahydrocannabinol (Δ-9-THC), Δ-8-tetrahydrocannabinol (Δ-8-THC), cannabigerol (CBG), cannabichromene (CBC), cannabigerolic acid (CBGA), Cannabidiolic acid (CBDA), THC acid (THCA), cannabichromenic Acid (CBCA), Cannabidivarin (CBDV), tetrahydrocannabivarin (THCV), Cannabichromevarin (CBCV), Cannabichromerocin (CBCV), Cannabivarin (CBV), Cannabicitran (CBT), Cannabinol (CBN), Cannabicyclol (CBL), Cannabigerorcin (CBGO), Cannabinodiol (CBND).

In some embodiments, the terpene is selected from: β-caryophyllene, β-myrcene, linalool, α- and β-pinene, limonene, β-amyrin, eucalyptol, alpha-terpineol, valencene, geraniol (lemonol), β-elemene, bisabolol, ocimene, terpinolene, humulene, α-terpinene, camphene, fenchol, α-phellandrene, Δ3-carene, γ-cardinene, sabinene and cycloartenol.

In some embodiments, the flavonoid is selected from: kaempferol, quercetin, cannflavin A, cannflavin B, canniprene, luteolin, apigenin, orientin, β-sitosterol, vitexin, isovitexin, and chrysin.

In some embodiments, at least one comprises at least 2, at least 3, at least 5, at least 7, at least 8, or at least 10, or any value and range therebetween. Each possibility represents a separate embodiment of the invention.

In some embodiments, at least one comprises 1 to 7, 1 to 8, 1 to 10, 2 to 6, 3 to 9, 3 to 10, 4 to 7, or 4 to 9. Each possibility represents a separate embodiment of the invention.

In some embodiments, the at least on additional therapeutic compound is an anti-inflammatory drug, an anti-microglial drug, or a combination thereof. In some embodiments, the composition further comprises an anti-inflammatory drug, an anti-microglial drug, or a combination thereof.

As used herein, the term “anti-inflammatory drug” refers to any compound capable of reducing or inhibiting an inflammatory response. As used herein, the term “anti-microglial drug” refers to any compound capable of inhibiting a microglia cell. In some embodiments, an anti-microglial cell reduces microglial cell’s activity and/or secretion, suppresses microglial cell signaling, or any combination thereof.

In some embodiments, an anti-inflammatory drug or an anti-microglial drug is selected from: an antibiotic, a tumor necrosis factor α (TNFα) inhibitor/blocker, a non-steroidal anti-inflammatory drug (NSAID), or an antidepressant drug.

In one embodiment, an antibiotic is minocycline.

In some embodiments, a TNFα inhibitor/blocker is selected from: infliximab, adalimumab, certolizumab pegol, golimumab, and etanercept. In some embodiments, a TNFα inhibitor/blocker is thalidomide or xanthine.

In some embodiments, a NSAID is selected from: celecoxib, aspirin, diclofenac, diflunisal, etodolac, ibuprofen, indomethacin, or acetaminophen.

In some embodiments, an antidepressant drug is selected from: ketamine, amitriptyline, amoxapine, desipramine, doxepin, imipramine, nortriptyline, protriptyline, or trimipramine.

In some embodiments, the pharmaceutical composition comprises a whole plant cannabis extract.

In some embodiments, the composition comprises a high CBD cannabis plant extract.

In some embodiments, the composition comprises a whole plant cannabis extract comprising CBD and at least one cannabinoid being different from CBD.

In some embodiments, the at least one cannabinoid being different from CBD is THC.

In some embodiments, the at least one cannabinoid being different from CBD is an ultra-low dose of Δ-9-tetrahydrocannabinol (THC). In some embodiments, an ultra-low dose is 0.001-0.02 mg.

In some embodiments, the composition comprises a whole plant cannabis extract comprising CBD and THC, to which exogenous CBD, either synthetic or plant-derived, is added to increase the CBD:THC ratio.

In some embodiments, the composition comprises whole cannabis extracts derived from any strain selected from: Ringo’s Gift, or Harle-Tsu (both by Southern Humboldt Seed Collective), or ACDC strain, or Cannatonic strain, or Dieseltonic or Hammershark (all four by Resin Seeds), or Charlotte’s web (by Charlotte’s Web Holdings), or Felina 32, or Santhica 27, or Futura 75, or Antal, or Kompolti Hybrid-TC, or Kompolti, or KC Dóra (all seven by Hempoint, s.r.o.), or Avidekel, or Metatron or Michael, or Rephael (all four by Tikun Olam), or EpiOne Oil, or Cure EP (both by Better), or Axiban (by Rafa), or DG (by MediCane), or OG Kush, or Tachllta Till (by Seach), or Remedy (by Remedy), or Berry Blossom, or Queen Dream, or Cherry blossom, or Cobbler Hemp, or Wife Hemp, or Chardonnay Hemp or Cherry Wine all seven by Blue Forest Farms), or Elektra, or Lifter (both by Oregon CBD), or Fermion, or Hindu Kush, or Gliana, or Jack Herer, or Jubileu, or Monoica, or Carmagnola, or Tisza, or Markant, or Fewdora 17, or Silvana, or Dacia, or CS Selected Carmagnola, or KC Zuzana, or Ratza, or Finola, or Eletta Campana, or Tiborszallasi, or Cinex, or Harlequin, or Granddaddy Purple, or XJ-13, or Blackberry Kush, or Girl Scout Cookies (GSC), or Sour Diesel, or Lamb’s Bread, or Pineapple Express, or Northern Lights, or Lavender, or Sour Tsunami.

In some embodiments, a whole plant cannabis extract comprises CBD and THC at a ratio ranging from 30:1 (w/w) to 1:1 (w/w).

In some embodiments, a whole plant cannabis extract consists of CBD and THC at a ratio ranging from 30:1 (w/w) to 1:1 (w/w).

In some embodiments, a ratio ranging from 30:1 (w/w) to 1:1 (w/w) comprises 28:1 (w/w) to 1:1 (w/w), 26:1 (w/w) to 1:1 (w/w), 24:1 (w/w) to 1:1 (w/w), 22:1 (w/w) to 1:1 (w/w), 20:1 (w/w) to 1:1 (w/w), 19:1 (w/w) to 1:1 (w/w), 18:1 (w/w) to 1:1 (w/w), 16:1 (w/w) to 1:1 (w/w), 14:1 (w/w) to 1:1 (w/w), 12:1 (w/w) to 1:1 (w/w), 11:1 (w/w) to 1:1 (w/w), 10:1 (w/w) to 1:1 (w/w), 9:1 (w/w) to 1:1 (w/w), 8:1 (w/w) to 1:1 (w/w), 7:1 (w/w) to 1:1 (w/w), 6:1 (w/w) to 1:1 (w/w), 5:1 (w/w) to 1:1 (w/w), 4:1 (w/w) to 1:1 (w/w), 3:1 (w/w) to 1:1 (w/w), or 2:1 (w/w) to 1:1 (w/w), or any value and range therebetween. Each possibility represents a separate embodiment of the invention.

In some embodiments, the CBD and THC are in a ratio ranging from 30:1 (w/w) to 1:1 (w/w), 28:1 (w/w) to 1:1 (w/w), 26:1 (w/w) to 1:1 (w/w), 24:1 (w/w) to 1:1 (w/w), 22:1 (w/w) to 1:1 (w/w), 20:1 (w/w) to 1:1 (w/w), 19:1 (w/w) to 1:1 (w/w), 18:1 (w/w) to 1:1 (w/w), 16:1 (w/w) to 1:1 (w/w), 14:1 (w/w) to 1:1 (w/w), 12:1 (w/w) to 1:1 (w/w), 11:1 (w/w) to 1:1 (w/w), 10:1 (w/w) to 1:1 (w/w), 9:1 (w/w) to 1:1 (w/w), 8:1 (w/w) to 1:1 (w/w), 7:1 (w/w) to 1:1 (w/w), 6:1 (w/w) to 1:1 (w/w), 5:1 (w/w) to 1:1 (w/w), 4:1 (w/w) to 1:1 (w/w), 3:1 (w/w) to 1:1 (w/w), 2:1 (w/w) to 1:1 (w/w), 5:1 (w/w) to 1:5 (w/w), 2:1 (w/w) to 1:2 (w/w), or any value and range therebetween, with a. Each possibility represents a separate embodiment of the invention.

The term “pharmaceutically acceptable” means suitable for administration to a subject, e.g., a human. For example, the term “pharmaceutically acceptable” can mean approved by a regulatory agency of the Federal or a state government or listed in the U. S. Pharmacopeia or other generally recognized pharmacopeia for use in animals, and more particularly in humans. The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The composition, if desired, can also contain minor amounts of wetting or emulsifying agents, or pH buffering agents such as acetates, citrates, or phosphates. Antibacterial agents such as benzyl alcohol or methyl parabens; antioxidants such as ascorbic acid or sodium bisulfite; and agents for the adjustment of tonicity such as sodium chloride or dextrose are also envisioned.

Non-limiting examples for suppressive microglia formulations include, but are not limited to, a formulation of CBD and THC; CBD and ultra-low THC; CBD and kaempferol; CBD and quercetin; CBD, β-caryophyllene and chrysin; CBD, linalool and quercetin; CBD, β-caryophyllene, linalool, kaempferol and quercetin; CBD, β-caryophyllene and kaempferol; CBD, linalool and kaempferol; CBD, ultra-low THC, and kaempferol; CBD, ultra-low THC, and quercetin; CBD, THC, β-caryophyllene, and kaempferol; and others.

The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. Although the bioavailability of peptides administered by other routes can be lower than when administered via parenteral injection, by using appropriate formulations it is envisaged that it will be possible to administer the compositions of the invention via transdermal, oral, rectal, vaginal, topical, nasal, inhalation and ocular modes of treatment. In addition, it may be desirable to introduce the pharmaceutical compositions of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

In some embodiments, the route of administration is based on a dissolving thin-film drug delivery system, prepared using hydrophilic polymers, or oral drug strip to administer drugs via absorption in the mouth (buccally or sublingually) and/or via the small intestines (enterically).

In some embodiments, the route of administration is improved by encapsulating the pharmaceutical agent in nanoparticles, such as to protect the encapsulated drug from biological and/or chemical degradation, and/or to facilitate transport to the brain thereby targeting microglia.

In some embodiments, the route of administration is improved by Pro NanoLipospheres (PNL) formulation or by PNLs with an incorporated natural absorption enhancer, such as piperine, curcumin and resveratrol.

In some thin-film drug delivery uses a dissolving film or oral drug strip to administer drugs via absorption in the mouth (buccally or sublingually) and/or via the small intestines (enterically). A film is prepared using hydrophilic polymers

In one embodiment, compositions of the present invention comprise compounds used for attenuating depression condition or disease in a subject in need thereof. In some embodiments, composition of the present invention is used in combination with electroconvulsive therapy.

In some embodiments, compositions for use according to the methods of this invention comprise solutions or emulsions, which in some embodiments are aqueous solutions or emulsions comprising a safe and effective amount of the compounds of the present invention and optionally, other compounds, intended for topical intranasal administration. In some embodiments, the compositions comprise from about 0.01% to about 10.0% w/v of a subject compound, or from about 0.1% to about 2.0, which is used for systemic delivery of the compounds by the intranasal route.

In another embodiment, the pharmaceutical compositions are administered by intravenous, intra-arterial, or intramuscular injection of a liquid preparation. In some embodiments, liquid formulations include solutions, suspensions, dispersions, emulsions, oils, and the like. In one embodiment, the pharmaceutical compositions are administered intravenously, and are thus formulated in a form suitable for intravenous administration. In another embodiment, the pharmaceutical compositions are administered intra-arterially, and are thus formulated in a form suitable for intra-arterial administration. In another embodiment, the pharmaceutical compositions are administered intramuscularly, and are thus formulated in a form suitable for intramuscular administration.

Further, in another embodiment, the pharmaceutical compositions are administered topically to body surfaces, and are thus formulated in a form suitable for topical administration. Suitable topical formulations include gels, ointments, creams, lotions, drops and the like. For topical administration, the compounds of the present invention are combined with an additional appropriate therapeutic agent or agents, prepared, and applied as solutions, suspensions, or emulsions in a physiologically acceptable diluent with or without a pharmaceutical carrier.

In one embodiment, pharmaceutical compositions of the present invention are manufactured by processes well known in the art, e.g., by means of conventional mixing, dissolving, granulating, dragee-making, levigating, emulsifying, encapsulating, entrapping or lyophilizing processes.

In one embodiment, pharmaceutical compositions for use in accordance with the present invention is formulated in conventional manner using one or more physiologically acceptable carriers comprising excipients and auxiliaries, which facilitate processing of the active ingredients into preparations which, can be used pharmaceutically. In one embodiment, formulation is dependent upon the route of administration chosen.

In one embodiment, injectables of the invention are formulated in aqueous solutions. In one embodiment, injectables, of the invention are formulated in physiologically compatible buffers such as Hank’s solution, Ringer’s solution, or physiological salt buffer. In some embodiments, for transmucosal administration, penetrants appropriate to the barrier to be permeated are used in the formulation. Such penetrants are generally known in the art.

In one embodiment, the preparations described herein are formulated for parenteral administration, e.g., by bolus injection or continuous infusion. In some embodiments, formulations for injection are presented in unit dosage form, e.g., in ampoules or in multi-dose containers with optionally, an added preservative. In some embodiments, compositions are suspensions, solutions, or emulsions in oily or aqueous vehicles, and contain formulatory agents such as suspending, stabilizing and/or dispersing agents.

The compositions also comprise, in some embodiments, preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcysteine, sodium metabisulfite and others; aromatic agents; viscosity adjustors, such as polymers, including cellulose and derivatives thereof; and polyvinyl alcohol and acid and bases to adjust the pH of these aqueous compositions as needed. The compositions also comprise, in some embodiments, local anesthetics or other actives. The compositions can be used as sprays, mists, drops, and the like.

In some embodiments, pharmaceutical compositions for parenteral administration include aqueous solutions of the active preparation in water-soluble form. Additionally, suspensions of the active ingredients, in some embodiments, are prepared as appropriate oily or water-based injection suspensions. Suitable lipophilic solvents or vehicles include, in some embodiments, fatty oils such as sesame oil, or synthetic fatty acid esters such as ethyl oleate, triglycerides or liposomes. Aqueous injection suspensions contain, in some embodiments, substances, which increase the viscosity of the suspension, such as sodium carboxymethyl cellulose, sorbitol or dextran. In another embodiment, the suspension also contains suitable stabilizers or agents which increase the solubility of the active ingredients to allow for the preparation of highly concentrated solutions.

In another embodiment, the active compound can be delivered in a vesicle, in particular a liposome (see Langer, Science 249:1527-1533 (1990); Treat et al., in Liposomes in the Therapy of Infectious Disease and Cancer, Lopez- Berestein and Fidler (eds.), Liss, New York, pp. 353-365 (1989); Lopez-Berestein, ibid., pp. 317-327; see generally ibid).

In another embodiment, the pharmaceutical composition delivered in a controlled release system is formulated for intravenous infusion, implantable osmotic pump, transdermal patch, liposomes, or other modes of administration. In one embodiment, a pump is used (see Langer, supra; Sefton, CRC Crit. Ref. Biomed. Eng. 14:201 (1987); Buchwald et al., (1980); Saudek et al., (1989). In another embodiment, polymeric materials can be used. In yet another embodiment, a controlled release system can be placed in proximity to the therapeutic target, thus requiring only a fraction of the systemic dose (see, e.g., Goodson, in Medical Applications of Controlled Release, supra, vol. 2, pp. 115-138 (1984). Other controlled release systems are discussed in the review by Langer (1990).

In some embodiments, the active ingredient is in powder form for constitution with a suitable vehicle, e.g., sterile, pyrogen-free water-based solution, before use. Compositions are formulated, in some embodiments, for atomization and inhalation administration. In another embodiment, compositions are contained in a container with attached atomizing means.

In one embodiment, the preparation of the present invention is formulated in rectal compositions such as suppositories or retention enemas, using, e.g., conventional suppository bases such as cocoa butter or other glycerides.

In some embodiments, pharmaceutical compositions suitable for use in context of the present invention include compositions wherein the active ingredients are contained in an amount effective to achieve the intended purpose. In some embodiments, a therapeutically effective amount means an amount of active ingredients effective to prevent, alleviate or ameliorate symptoms of disease or prolong the survival of the subject being treated.

In one embodiment, determination of a therapeutically effective amount is well within the capability of those skilled in the art.

In some embodiments, preparation of effective amount or dose can be estimated initially from in vitro assays. In one embodiment, a dose can be formulated in animal models and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient’s condition. [See e.g., Fingl, et al., (1975)].

In one embodiment, dosing can be of a single or a plurality of administrations, with course of treatment lasting from several days to several weeks or until cure is affected or diminution of the disease state is achieved. In another embodiment, said dosing can depend on severity and responsiveness of the condition to be treated.

In one embodiment, the amount of a composition to be administered will, of course, be dependent on the subject being treated, the severity of the affliction, the manner of administration, the judgment of the prescribing physician, etc.

In one embodiment, compositions including the preparation of the present invention formulated in a compatible pharmaceutical carrier are also be prepared, placed in an appropriate container, and labeled for treatment of an indicated condition.

In some embodiments, the term “therapeutically effective amount” refers to a concentration of at least one cannabinoid, at least one terpene, and at least one flavonoid, effective to treat a disease or disorder in a mammal. The term “a therapeutically effective amount” refers to an amount effective, at dosages and for periods of time necessary, to achieve the desired therapeutic result. The exact dosage form and regimen would be determined by the physician according to the patient’s condition.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

It is to be noted that the term “a” or “an” entity, refers to one or more of that entity; for example, “a cannabinoid,” is understood to represent one or more cannabinoids. As such, the terms “a” (or “an”), “one or more,” and “at least one” can be used interchangeably herein.

Throughout this specification and claims, the word “comprise,” or variations such as “comprises” or “comprising,” indicate the inclusion of any recited integer or group of integers but not the exclusion of any other integer or group of integers.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises,” “comprising, “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization – A Laboratory Course Manual” CSHL Press (1996); all of which are incorporated by reference. Other general references are provided throughout this document.

### Example 1

### Formulations of Cannabinoid(s) With Terpene(s) and/or Flavonoid(s) and/or Celecoxib Have Superior Microglia-Suppressing Properties in Vitro

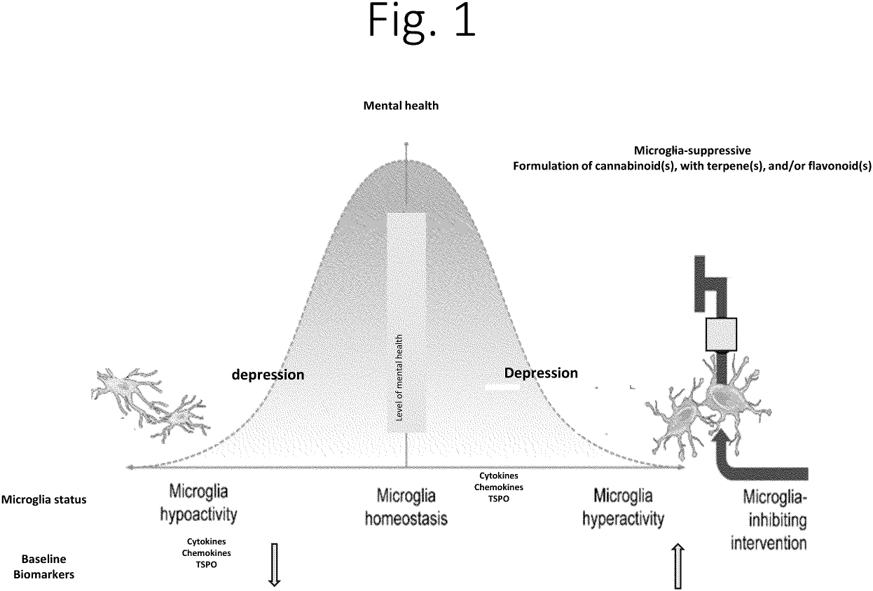

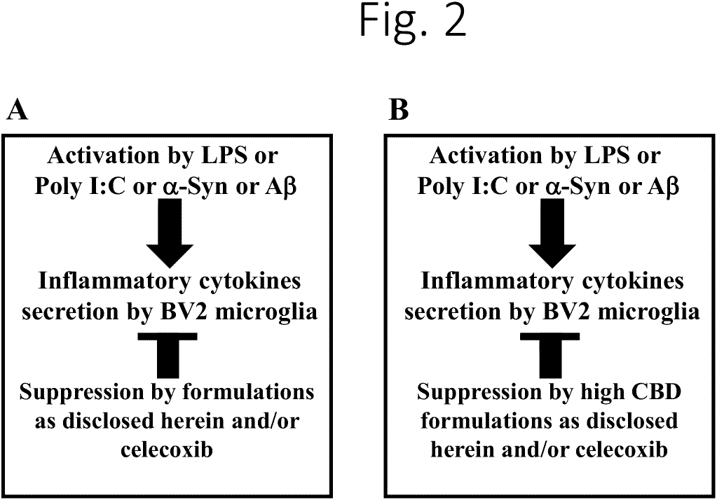

The current invention is directed to formulations comprising at least one cannabinoid, with at least one additional cannabinoid, and/or terpene, and/or flavonoid, and/or celecoxib, having superior microglia-suppressive effects, compared to the effects of an individual cannabinoid, terpene, flavonoid or celecoxib. These superior effects are demonstrated by systematically examining the microglia-suppressing effects of at least one cannabinoid, formulated with at least one additional cannabinoid, and/or terpene and/or flavonoid, and/or celecoxib, or a combination thereof, in cultures of activated microglia cells. Such formulations are utilized in a personalized medical approach for treating or attenuating a mood disorder in a subject having a high inflammatory/microglial state (FIG. 1). The BV2 murine microglial cell line is used in these studies. These cells exhibit most of the morphological and functional properties of primary isolated microglia, including the production of inflammatory cytokines upon exposure to specific immune challenges, thus serving as cellular models of various microglial-associated neuropsychiatric pathologies. Such challenges can include: 1) lipopolysaccharide (LPS or endotoxin; the cell-wall component of Gram-negative bacteria), which mimics bacterial infection and served as the most useful model for studying microglia activation; 2) Polyinosinic:polycytidylic acid (Poly I:C), a synthetic analog of double-stranded RNA that serves as a model for viral-induced microglia activation, 3) α-synuclein, a protein whose aggregates as fibrils stimulate microglia in Parkinson’s disease and other synucleinopathies; 4) β-amyloid, a peptide comprising the main component of the amyloid plaques in the brains of Alzheimer’s disease patients, and the major stimulator of microglia in this disease (FIG. 2).

The BV2 cells are cultured at 37° C. in a humidified atmosphere with 5% CO2 in high D-glucose (4.5 g/L) Dulbecco’s modified Eagle’s medium (Sigma, Rehovot, Israel) supplemented with 5% heat-inactivated fetal bovine serum, streptomycin (100 µg/ml), and penicillin (100 units/ml) and sodium pyruvate (1 mM; all from Biological Industries Ltd., Kibbutz Beit Haemek, Israel). 24 hours before experiments with LPS stimulation, the BV2 cells are split into 24 well plates, 2×10⁵ cells per well, covered with 1 ml of growth medium and allowed to attach overnight. 24 hr before experiments with α-synuclein, the BV2 cells are split into 96-well plates, 50,000 (5×10⁴) cells per well, covered with 200 µl of growth medium and allowed to attach overnight. Cultures of BV2 microglia cells are first treated by an individual cannabinoid, or terpene or flavonoid selected from: Cannabidiol (CBD), Δ-9-tetrahydrocannabinol (THC), Δ-8-tetrahydrocannabinol (THC), cannabigerol (CBG), cannabichromene (CBC), cannabigerolic acid (CBGA), Cannabidiolic acid (CBDA), THC acid (THCA), cannabichromenic Acid (CBCA), Cannabidivarin (CBDV), tetrahydrocannabivarin (THCV), Cannabichromevarin (CBCV), Cannabichromerocin (CBCV), Cannabivarin (CBV), Cannabicitran (CBT), Cannabinol (CBN), Cannabicyclol (CBL), Cannabigerorcin (CBGO), Cannabinodiol (CBND), β-caryophyllene, β-elemene, β-myrcene, linalool, α-pinene, β-pinene, eucalyptol, alpha-terpineol, valencene, geraniol (lemonol), bisabolol, nerolidol, ocimene, terpinolene, humulene, α-terpinene, camphene, fenchol, α-phellandrene, Δ3-carene, γ-cardinene, sabinene, limonene, β-amyrin, cycloartenol, cannflavin A, cannflavin B, kaempferol, quercetin, luteolin, apigenin, canniprene, orientin, β-sitosterol, vitexin, isovitexin, and chrysin. Additional cultures of BV2 microglia cells are treated by an individual cannabinoid, with at least one additional cannabinoid, and/or terpene, and/or flavonoid, and/or celecoxib. Additional cultures are exposed to the vehicle of these compounds (cremophor, ethanol and PBS, 1:1:18 (v/v/v)) (FIG. 2A). In some studies, cultures of BV2 microglia cells are treated by a specific whole plant cannabis extract, with or without supplementation with cannabinoid(s), and/or terpene(s) and/or flavonoid(s), and/or celecoxib (FIG. 2B).

For each individual cannabinoid mentioned above, 3-4 concentrations are used, ranging from 0.1 to 10 µM. For ultra-low dose Δ9-THC the concentration used is 0.01 µM. For each individual terpene or flavonoid mentioned above, 3-4 concentrations are used, ranging from 1 to 50 µM. For celecoxib, 3-4 concentrations are used, ranging from 0.1 to 10 µM. Two hours following the treatment, the cultures are exposed to one of the following microglia stimulators: LPS (Sigma L2630; Escherichia coli serotype O111:B4) is dissolved in D-PBS (Sigma, D8537) to a pre-stock concentration of 1 mg/ml, aliquoted and stored at -80° C. Stock solution of LPS at 10 ug/ml is prepared using sterile D-PBS, followed by filtering via 0.22 µm filter membrane, aliquoting and storing in -20° C. LPS is applied into the cell medium at a final concentration of 100 ng/ml. Poly I:C (Sigma, P1530) was first dissolved in sterile, molecular grade water to the concentration of 10 mg/ml (following the manufacturer’s instructions). This stock solution was further diluted to the concentration of 1 mg/ml in sterile PBS, and the aliquots stored in -20oC. The final concentration of Poly I:C in the cell medium used to activate the BV2 cells was 10 µg/ml, and was chosen based on the pilot concentration- and time-dependent experiments. α-Synuclein (lyophilized recombinant human, Sigma, S7820) is reconstituted in sterile, molecular grade water to the concentration of 50 µM (1 mg/ml). To induce oligomerization, α-Synuclein solution is placed for 24 hours on a shaker with agitation of 80 rpm and at 37° C. Following verification of oligomerization efficiency (using ELISA assay for TNFα cytokine concentration and native gel electrophoresis), the oligomerized α-Synuclein preparations are aliquoted and stored in -20° C. A concentration of 500 nM α-Synuclein is used in the experiments.

Four or 24 hours following the initiation of the microglia stimulating challenge exposure, the culture media is collected and spun down for 5 min at 2,000 rpm. Cell free media is frozen in -80° C. till further analysis._The concentrations of TNFα, IL-6 and IL-1β in the cell conditioned and cell-free media are determined using mouse ELISAs following the protocols recommended by the supplier (R&D Systems, Minneapolis, MN, USA). The serum in the culture media do not interfere with the assays. Frozen cell free media are thawed on the ice-cold surface, vortexed and proceeded for ELISA assays. The ELISA measurements are carried out using Tecan Sunrise absorbance plate reader with 450 nm filter (and 620 nm filter background correction) and the data (OD values) processed using Magellan TM and MS Excel software. The raw OD values obtained using absorbance microplate reader are transformed to relative % values, with the OD value obtained for LPS, without drug pre-treatments, assigned as 100% activation (maximal inflammatory response). The effects of the drug treatments are then compared to the Vehicle plus LPS group and accordingly expressed as % values. The statistical analysis is performed on % values, on N=3-4 samples. The Graph Pad Prism software v9.0.2. (San Diego, CA, USA) is used for statistical analysis. The data are expressed as the mean ± S.E.M. and analyzed for statistical significance using one-way analysis of variance (ANOVA), followed by Dunnett’s post-hoc tests for comparisons to the vehicle-LPS group, and Tukey’s post hoc test for comparisons of each combination of drugs vs. the effect of each component given individually in the corresponding concentration. p <0.05 was considered significant.

A formulation of the most effective microglia-suppressive cannabinoid(s) with terpene(s), and/or flavonoid(s) is selected for further assessment and comparison to the individual components of the formulation or to other formulations. The most effective cannabinoid, terpene or flavonoid is defined as the molecule that produced a significant microglia suppression at the lowest concentration in each class of molecules, and within this concentration produced the largest % decline in cytokine levels (as compared with LPS/PolyI:C/α-Syn/aβ)-treated cultures with vehicle only). The second and third most effective cannabinoid, terpene or flavonoid are also selected in this analysis.

Formulations of the most effective cannabinoid(s) with terpene(s), and/or flavonoid(s) are systematically tested for their ability to suppress LPS- or PolyI:C- or α-Syn-or amyloid-β-induced BV2 microglia activation, in a systematic order, as described above. According to this non-limiting example, the cannabinoids are CBD and ultra-low THC, the terpenes are BCP and linalool, and the flavonoids are kaempferol and quercetin. Non-limiting method for such systematic examination is as follows:

The microglia-suppressive effects of CBD and ultra-low THC are tested. 1. The single and pairs of these cannabinoids are each tested first with one terpene (i.e., BCP or linalool) by itself, then with the pair combination of these terpenes. 2. The single and pairs of cannabinoids are each tested first with one flavonoid (i.e., kaempferol or quercetin) by itself, then with the pair combination of these flavonoids. 3. The single and pairs of cannabinoids are tested with single and pairs of terpenes and single and pairs combinations of flavonoids (for example, one such combination is CBD and kaempferol; another example is CBD and quercetin; another example is CBD, ultra-low THC and BCP; another example is CBD, ultra-low THC, linalool and kaempferol). Complex combinations such as these ones are tested for microglia suppression and compared to the lower-order combinations that comprise them. 4. In subsequent experiments, higher-order combinations (with additional cannabinoids, terpenes and flavonoids), for example CBD, α-pinene, β-pinene, (E)-β-ocimene, BCP, geraniol, germacrene-D, luteolin, kaempferol and quercetin) are similarly tested and compared to lower-order combinations.

### Example 2

### Effects of Formulations of Cannabinoids, Terpenes, Flavonoids, and Their Combinations on LPS-Induced TNFα Production by BV2 Microglia Cell Cultures

As a preliminary investigation of the approach presented in Example 1, the inventors treated cultures of BV2 microglia cells with various cannabinoids, terpenes, flavonoids and their combinations, then added LPS to these cultures, and following a 24-hours incubation period, measured the concentrations of TNFα in the cultures’ medium. In this preliminary study we used the methods described in Example 1 to examine the effects of relatively low concentrations of the cannabinoids cannabidiol (CBD), Δ-9 THC, Cannabidiolic acid (CBDA), and cannabichromene (CBC), the terpenes β-caryophyllene (BCP), α-pinene, β-pinene, limonene, and linalool, the flavonoids kaempferol (Kae) and quercetin (Quer), as well as some combinations of these compounds.

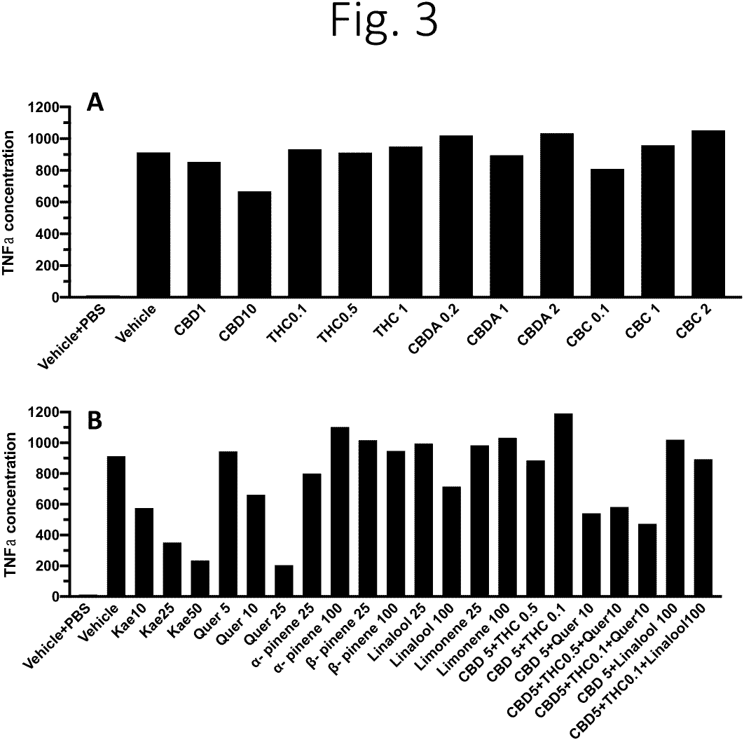

BV2 cells treated with saline served as control, showing no detectable levels of TNFα. Compared to cultures treated with vehicle and LPS, the levels of TNFα were reduced by CBD (10 but not 1 µM), linalool (100 but not 25 µM), kaempferol (dose-dependently at 10, 25 and 50 µM) and quercetin (dose-dependently by 10 and 25, but not by 5 µM) (FIGS. 3A, 3B). Combination of CBD (5 µM) and quercetin (10 µM) produced a suppressive effect, which was greater than the effect of each individual component. Combination of CBD (5 µM), THC (0.1 µM) and quercetin (10 µM) produced the most potent microglia suppression.

### Example 3

### Effects of CBD on LPS-, Poly I:C- and Α-Synuclein-Induced Cytokine Production by BV2 Microglia Cultures

Ample previous evidence demonstrated the anti-inflammatory and microglia-suppressive effects of CBD in various cellular models, particularly using the bacterial component lipopolysaccharide (LPS). In the current Example we corroborate the effects of CBD in the LPS model, and extend these findings to demonstrate the microglia-suppressive effects of CBD in a model of neuroinflammation induced by the viral mimetic Poly I:C, as well as a model of microglia activation induced by aggregated α-synuclein. This compound, which is the major component of Lewy body, is the pathological hallmark of Parkinson’s disease, Lewy body dementia and other synucleinopathies. α-synuclein is particularly implicated in microglia activation and neuroinflammation that are causally associated with Parkinson’s disease progression.

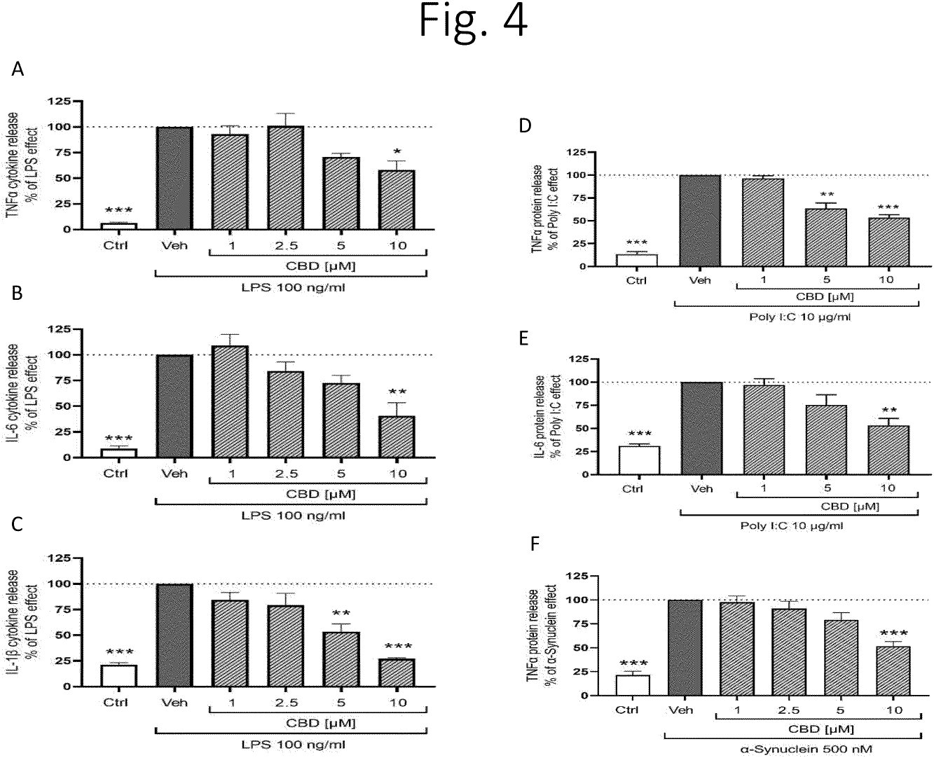

FIG. 4 depicts the effects of CBD at the concentrations 1-10 µM on the release of several cytokines by BV2 microglial cells activated with either with LPS (A-C), or with Poly I:C (D-E), or with α-synuclein aggregates/fibrils (E). Analysis of the effect of CBD on the LPS-induced microglial activation revealed that CBD (10 µM) significantly inhibited the release of TNFα (F_{5,12}=26.54; p<0.001) and of IL-6 (F_{5,12}=20.88; p<0.001), while 5 and 10 µM effectively reduced IL-1β levels (F_{5,12}=25.16; p<0.001). Similarly, CBD (5 and 10 µM) significantly decreased Poly I:C-induced TNFα release (F₄, ₁₀= 96.49, p<0.001) while 10 µM markedly decreased IL-6 (F_{4,10}=18.55, p<0.001). Finally, CBD (10 µM) reduced the secretion of TNFα cytokine in microglial cells incubated with α-synuclein aggregates/fibrils (F_{5,12}= 28.92, p<0.001). These results suggest that CBD exhibit comparable potency in various in vitro models of neuroinflammation.

### Example 4

### Synergistic Effects of CBD and Kaempferol on LPS-Induced TNFα and IL-1β Production by BV2 Microglia Cultures

Kaempferol, which is one of the major flavonoids found in cannabis chemovars, has been previously found to exert anti-inflammatory and microglia suppressive effects. To corroborate and extend the findings of Example 2, we examined the effects of CBD, kaempferol and their combinations on LPS-induced proinflammatory cytokine secretion by BV2 cells, using the methodology described in Example 1.

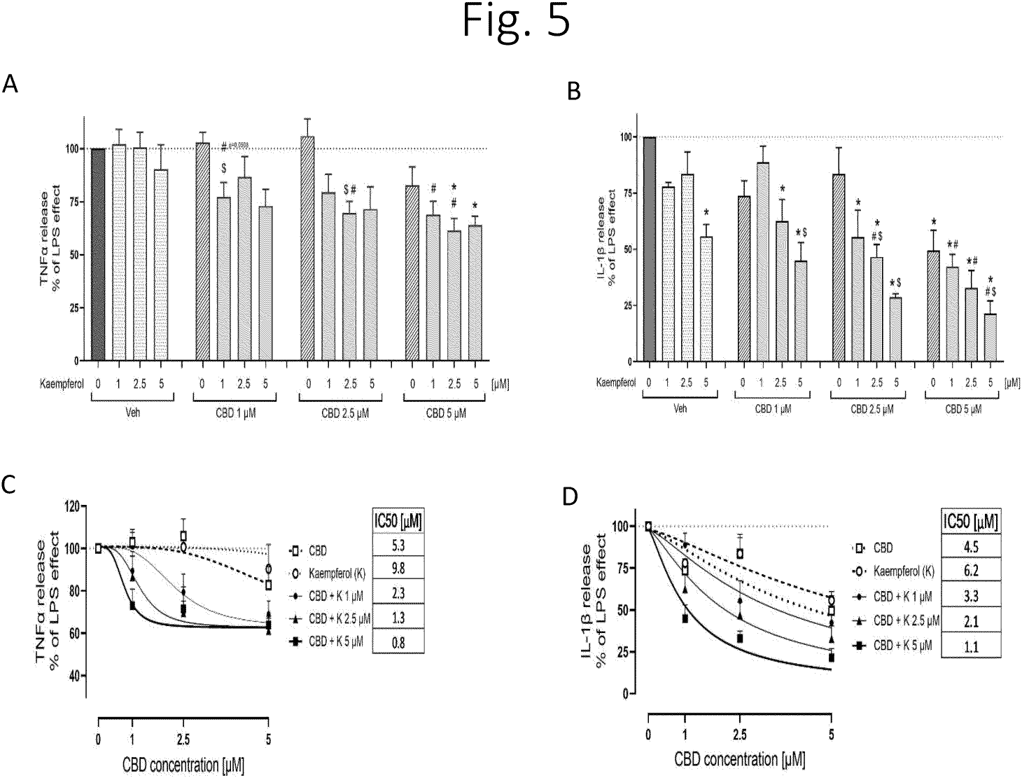

Analysis of the effects of kaempferol, CBD and their combinations on LPS-induced TNFα secretion demonstrated a significant difference between the concentrations of these compounds (F_{15,48}=4.09, p<0.001) (FIG. 5A). Post-hoc tests reveal that by themselves, neither CBD nor kaempferol (each at concentrations of 1, 2.5 and 5 µM) produced significant inhibitory effects. However, combinations of CBD (5 µM) plus kaempferol (at either 2.5 or 5 µM) significantly decrease the release of TNFα, compared with the effect observed in LPS-activated cultures treated with Veh (p<0.01 and p<0.05, respectively). Furthermore, comparisons of the effects of each combination with its components given by themselves revealed the following differences: 1) CBD (1 µM) plus Kaempferol (1 µM) produced stronger TNFα suppression than CBD (p<0.05) or kaempferol (p = 0.0508) at 1 µM. 2) Combination of CBD (2.5 µM) plus kaempferol (2.5 µM) produced stronger TNFα suppression than CBD or kaempferol, given by themselves at 2.5 µM (both p<0.05). 3) Combinations of CBD (5 µM) plus kaempferol (at either 1 or 2.5 µM) produced stronger TNFα suppression than kaempferol applied by itself at these concentrations (p<0.05).

For a more global analysis of these findings, an XY plot was prepared, where the drugs concentrations are plotted on the x-axis and the response to the drug, i.e., TNFα release, expressed as % of maximal LPS effect, is plotted on the y-axis. The XY plot shows nonlinear regression curves fitted based on wide-range concentration-response experiments of CBD and kaempferol tested individually, or when kaempferol (1 or 2.5 or 5 µM) was added to various concentrations of CBD (FIG. 5C). Calculation of the Inhibitory Concentration (IC) 50 values for CBD, kaempferol and their combinations are presented in the Table adjacent to the XY plot, demonstrating that the IC50s for CBD and kaempferol by themselves were 5.3 µM and 9.8 µM, respectively, i.e., kaempferol is nearly twice less potent in inhibiting TNFα release than CBD. Addition of 1, 2.5 or 5 µM of Kaempferol to each of the tested doses of CBD (1, 2.5 and 5 µM) decreased the CBD IC50 to 2.3 µM, 1.3 and 0.8, respectively.

Analysis of the effects of kaempferol, CBD and their combinations on LPS-induced IL-1β secretion demonstrated significant differences between the treatments (F_{15,} ₄₄=9.79, p<0.001; FIG. 5A). Post-hoc tests reveal that (1) neither CBD nor kaempferol (at concentrations of 1 or 2.5 µM) produced significant inhibitory effects, while 5 µM of either CBD or kaempferol were effective in decreasing IL-1β (p<0.001 and p<0.01, respectively), (2) addition of individually inactive 2.5 µM of kaempferol significantly potentiated the IL-1β inhibition by previously inactive 1 µM of CBD, as compared to Veh plus LPS group (p<0.01). Moreover, (3) addition of inactive doses of kaempferol 1 µM and of 2.5 µM to a non-effective dose of 2.5 µM of CBD, resulted in significantly decreased levels of IL-1β (p<0.01 and p<0.001, respectively). Furthermore, comparisons of the effects of each combination with its components given by themselves revealed the following differences: (1) CBD (1 µM) plus kaempferol (5 µM) resulted in stronger IL-1β suppression than CBD given alone at 1 µM (p < 0.05), (2) combination of CBD (2.5 µM) plus kaempferol (2.5 µM) was significantly stronger in decreasing IL-1β release than either CBD (2.5 µM) or kaempferol (2.5 µM), when given by themselves (p<0.05); (3) CBD (2.5 µM) plus kaempferol (5 µM) resulted in significantly stronger IL-1β inhibition than CBD alone (at 2.5 µM) (p<0.01); 4) Addition of kaempferol (1 µM or 2.5 µM) to CBD (5 µM) resulted in the inhibition of IL-1β levels that was significantly stronger than the respective doses of kaempferol tested alone (p<0.01).

For a more global analysis of these findings, an XY plot was prepared, where the drugs concentrations are plotted on the x-axis and the response to the drug, i.e., IL-1β release, expressed as % of maximal LPS effect, is plotted on the y-axis. The XY plot shows nonlinear regression curves fitted based on wide-range concentration-response experiments of CBD and kaempferol tested individually, or when kaempferol (1 or 2.5 or 5 µM) was added to various concentrations of CBD (FIG. 5D). Calculation of the Inhibitory Concentration (IC) 50 values for CBD, kaempferol and their combinations are presented in the Table adjacent to the XY plot, demonstrating that the IC50s for CBD and kaempferol by themselves were 4.5 µM and 6.2 µM, i.e., kaempferol is slightly less potent in inhibiting IL-1β release than cannabinoid CBD. Addition of 1, 2.5 or 5 µM of kaempferol to each of the tested doses of CBD (1, 2.5 and 5 µM) decreased the CBD IC50 to 3.3 µM, 2.1 µM and 1.1 µM, respectively.

Together, these findings demonstrate that CBD and kaempferol produce synergistic suppressive effects on microglia activation.

### Example 5

### Synergistic Effects of CBD and Quercetin on LPS-Induced TNFα and IL-1β Production by BV2 Microglia Cultures

Quercetin, which is another major flavonoid found in cannabis chemovars, has been previously found to exert potent anti-inflammatory and microglia suppressive effects. Quercetin was also found to produce antidepressant effects in animal models of depression, and it is a major component of plants other than cannabis, such as Hypericum perforatum (St. John’s Wort), turmeric, and Saffron, which were shown to produce antidepressant effects in clinical trials.

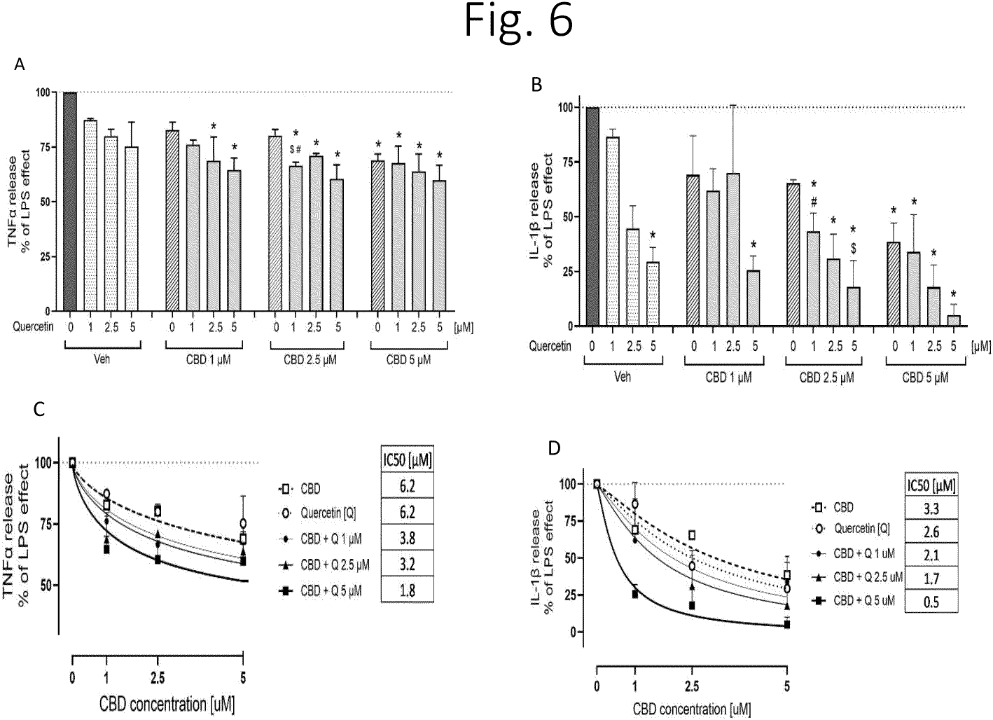

Analysis of the effects of quercetin, CBD and their combinations on LPS-induced TNFα secretion demonstrated significant differences between the treatments (F_{15,16}=3.498, p<0.01; FIG. 6A). Post-hoc tests revealed that (1) none of the tested concentrations of Quercetin (1, 2.5 or 5 µM) given alone affected the TNFα release (2) CBD (1 µM and 2.5 µM) given alone did not affect the TNFα release while CBD (5 µM) significantly (p<0.05) decreased the TNFα secretion from LPS activated microglial cells, (3) CBD (1 µM) plus quercetin (2.5 or 5 µM) led to significant inhibition of TNFα release (p<0.05 and p< 0.01, respectively, vs Veh plus LPS), (4) CBD (2.5 µM) plus quercetin (1 or 2.5 µM or 5 µM) led to significant inhibition of TNFα release (p<0.01, p<0.05 and p<0.01, respectively), (5) combining CBD (5 µM) with all three increasing concentrations of quercetin resulted in significant inhibition of TNFα release (p<0.05, p<0.01, p<0.01, respectively). Furthermore, comparisons of the effects of each combination with its components given by themselves revealed that combining CBD (2.5 µM) with quercetin (1 µM) resulted in inhibition of TNFα release that was significantly stronger than each of the compounds applied individually in the same concentrations (p<0.05 vs CBD alone and p<0.01 vs quercetin alone).

For a more global analysis of these findings, an XY plot was prepared, where the drugs concentrations are plotted on the x-axis and the response to the drug, i.e., TNFα release, expressed as % of maximal LPS effect, is plotted on the y-axis. The XY plot shows nonlinear regression curves fitted based on wide-range concentration-response experiments of CBD and quercetin tested individually, or when quercetin (1 or 2.5 or 5 µM) was added to various concentrations of CBD (FIG. 6C). Calculation of the Inhibitory Concentration (IC) 50 values for CBD, quercetin and their combinations are presented in the Table adjacent to the XY plot, demonstrating that the IC50s for CBD and quercetin by themselves were 6.2 µM and 6.2 µM, i.e., both compounds were equipotent in inhibiting TNFα release induced by LPS activation. Addition of 1, 2.5 or 5 µM of quercetin to each of the tested doses of CBD (1, 2.5 and 5 µM) decreased the CBD IC50 to 3.8 µM, 3.2 µM and 1.8 µM, respectively.

Analysis of the effects of quercetin, CBD and their combinations on LPS-induced IL-1β secretion demonstrated a significant difference between the treatments (F_{15,} ₁₆ = 4.707, p<0.01; FIG. 6B). Post-hoc tests revealed that (1) CBD (given individually at 1 µM and at 2.5 µM) had no effect on IL-1β release, but CBD (5 µM) decreased the IL-1β levels significantly (p<0.05) and that (2) quercetin alone had no effect at 1 and 2.5 µM, but 5 µM of this flavonoid significantly decreased IL-1β levels (p<0.01), (3) CBD (2.5 µM) plus Quercetin (1 µM) induced significant inhibition of IL-1β as compared to Veh plus LPS group (p<0.05), (4) addition of quercetin (2.5 µM) to CBD (2.5 or 5 µM) further enhanced the decrease in IL-1β (p<0.05 and p<0.01, respectively), (5) combination of CBD plus quercetin (each at 5 µM) decreased the levels of IL-1β to the baseline levels (p<0.001). Furthermore, comparisons of the effects of each combination with its components given by themselves revealed that (1) adding quercetin (1 µM) to CBD (2.5 µM) inhibited IL-1β significantly stronger than quercetin alone in the same dose (p<0.05) and (2) adding quercetin (5 µM) to CBD (2.5 µM) resulted in stronger IL-1β inhibition than CBD alone in the same dose (p<0.05).

For a more global analysis of these findings, an XY plot was prepared, where the drugs concentrations are plotted on the x-axis and the response to the drug, i.e., IL-1β release, expressed as % of maximal LPS effect, is plotted on the y-axis. The XY plot shows nonlinear regression curves fitted based on wide-range concentration-response experiments of CBD and quercetin tested individually, or when quercetin (1 or 2.5 or 5 µM) was added to various concentrations of CBD (FIG. 6D). Calculation of the Inhibitory Concentration (IC) 50 values for CBD, quercetin and their combinations are presented in the Table adjacent to the XY plot, demonstrating that the IC50s for CBD and quercetin by themselves were 3.3 µM and 2.6 µM. Addition of 1 or 2.5 or 5 µM of quercetin to each of the tested doses of CBD (1, 2.5 and 5 µM) decreased the CBD IC50 to 2.1 µM, 1.7 µM and 0.5 µM, respectively.

Together, these findings demonstrate that CBD and quercetin produce synergistic suppressive effects on microglia activation.

### Example 6

### Synergistic Effects of CBD and CBDV on LPS-induced TNFα, and IL-1β Production by BV2 Microglia Cultures

Data on the effects of the Cannabidivarin (CBDV), and the mechanisms underlying these effects, is limited. Still, there is evidence that cannabinoid can produce anti-inflammatory effects, exerting neuroprotective and therapeutic effects in models of epilepsy, pain and neurodevelopmental disorders.

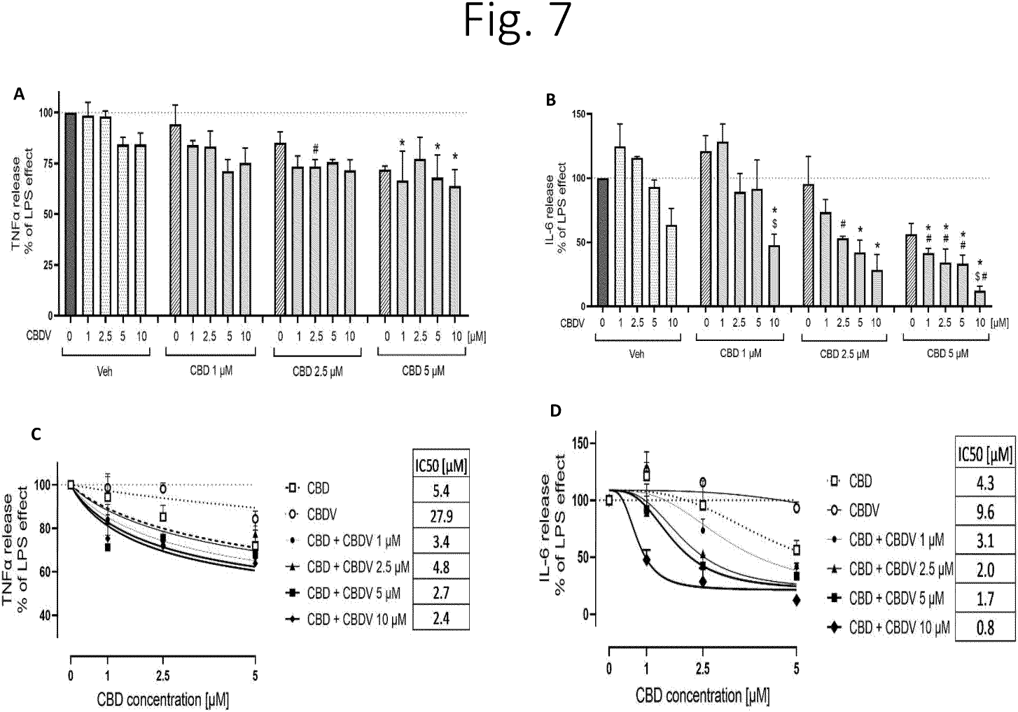

Analysis of the effects of CBD, CBDV and their combinations on LPS-induced TNFα secretion demonstrated a significant difference between the treatments (F_{19,40} = 2.586, p<0.01, FIG. 7A). Post-hoc tests reveal that (1) neither of the tested concentrations of CBDV (1, 2.5, 5 or 10 µM) given alone nor any of the tested concentrations of CBD (1, 2.5 or 5 µM) affected TNFα release from LPS activated microglial cells, (2) combination of CBD (5 µM) plus CBDV (1 or 5 or 10 µM) led to significant decreases in TNFα levels (p<0.05, p<0.05 and p<0.01, respectively). Furthermore, comparisons of the effects of each combination with its components given by themselves revealed that the combination of CBD (2.5 µM) plus CBDV (2.5 µM) was significantly stronger than CBDV (2.5 µM) applied alone (p<0.05).

For a more global analysis of these findings, an XY plot was prepared, where the drugs concentrations are plotted on the x-axis and the response to the drug, i.e., TNFα release, expressed as % of maximal LPS effect, is plotted on the y-axis. The XY plot shows nonlinear regression curves fitted based on wide-range concentration-response experiments of CBD and CBDV tested individually, or when CBDV (1 or 2.5, 5 or 10 µM) was added to various concentrations of CBD (FIG. 7C). Calculation of the Inhibitory Concentration (IC) 50 values for CBD, quercetin and their combinations are presented in the Table adjacent to the XY plot, demonstrating that the IC50s for CBD and CBDV by themselves were 5.4 µM and 27.9 µM, i.e., CBDV was nearly 6 times less potent (if at all) in inhibiting TNFα release induced by LPS activation. Addition of 1, 2.5, 5 or 10 µM of CBDV to each of the tested doses of CBD (1, 2.5 and 5 µM) decreased the CBD IC50 to 3.4 µM, 4.8 µM, 2.7 µM and 2.4 µM, respectively.

Analysis of the effects of CBDV, CBD and their combinations on LPS-induced IL-6 secretion demonstrated a significant difference between the treatments (F_{19,} ₄₀ = 9.603, p<0.001; FIG. 7B). Post hoc test revealed (1) no significant effect CBD or CBDV given aby themselves, but (2) combination of CBD (1 µM) plus CBDV (10 µM) or CBD (2.5 µM) plus CBDV (5 or 10 µM) resulted in significant IL-6 decreases (p<0.05 and p< 0.01, respectively), and that (3) combination of CBD (5 µM) with CBDV (1 or 2.5, or 5, or 10 µM) led to significant inhibitions of IL-6 release from activated microglia (p<0.05, p<0.01, p<0.01 and p<0.001, respectively). Furthermore, comparisons of the effects of each combination with its components given by themselves revealed that (1) combination of CBD (1 µM) plus CBDV (10 µM) was significantly stronger than CBD alone (p<0.01) and that (2) combination of CBD (2.5 µM) plus CBDV (2.5 µM) was significantly stronger than CBD alone (p<0.01), (3) combination of CBD (5 µM) plus CBDV (at either 1 or 5 µM) led to stronger IL-6 inhibition than CBDV applied individually in each of the corresponding concentrations (p<0.01), (4) combination of CBD (5 µM) plus CBDV (10 µM) resulted in IL-6 inhibition that was significantly stronger than each of the compounds given individually (both p<0.05).

For a more global analysis of these findings, an XY plot was prepared, where the drugs concentrations are plotted on the x-axis and the response to the drug, i.e., IL-6 release, expressed as % of maximal LPS effect, is plotted on the y-axis. The XY plot shows nonlinear regression curves fitted based on wide-range concentration-response experiments of CBD and CBDV tested individually, or when CBDV (1 or 2.5, 5 or 10 µM) was added to various concentrations of CBD (FIG. 7D). Calculation of the Inhibitory Concentration (IC) 50 values for CBD, quercetin and their combinations are presented in the Table adjacent to the XY plot, demonstrating that the IC50s for CBD and CBDV by themselves were 4.3 µM and 9.6 µM, i.e., CBDV was about 2 times less potent in inhibiting IL-6 release induced by LPS activation. Addition of 1, 2.5, 5 or 10 µM of CBDV to each of the tested doses of CBD (1, 2.5 and 5 µM) decreased the CBD IC50 to 3.1 µM,2.0 µM,1.7 µM and 0.8 µM,respectively.

Together, these findings demonstrate that CBD and quercetin produce synergistic suppressive effects on microglia activation.

### Example 7

### Synergistic Effects of CBD and Celecoxib on LPS-Induced TNFα, and IL-1β Production by BV2 Microglia Cultures

The cyclooxygenase (COX)-2 inhibitory drug Celecoxib exerts potent anti-inflammatory and microglia-suppressive effects, by blocking the production of prostaglandins, which are major drivers of inflammatory processes. Celecoxib has also been shown to produce antidepressant effects in animal models of depression, as well as in randomized clinical trials, particularly in depressed patients with elevated markers of inflammatory and microglial activation. Given that CBD produces microglia-suppressive and antidepressant effects via mechanisms that are complementary to those of celecoxib, we examined here the interactive effects of CBD and celecoxib on LPS-induced secretion of pro-inflammatory cytokines by microglia cells.

Celecoxib (Sigma, PHR1638) was purchased from Sigma Aldrich Ltd (Rehovot, Israel). CBD was purchased from BOL Pharma (Revadim, Israel). Celecoxib powder was stored in RT in desiccator compartment. Celecoxib and CBD solutions were prepared as 10 mM stock in pure absolute Ethanol 100% (BioLab Ltd, Jerusalem, Israel) and stored in -20° C. The working solutions used in in vitro studies at 1 mM were prepared in sterile cell growth medium just before the experiment. The maximal concentration of EtOH in the cell medium did not exceed 0.2% (mainly 0.1%), the concentration that did not interfere with cytokine release. Each drug was tested at the concentrations of 1, 2.5, 5 and 10 µM, alone or in combination with each other.

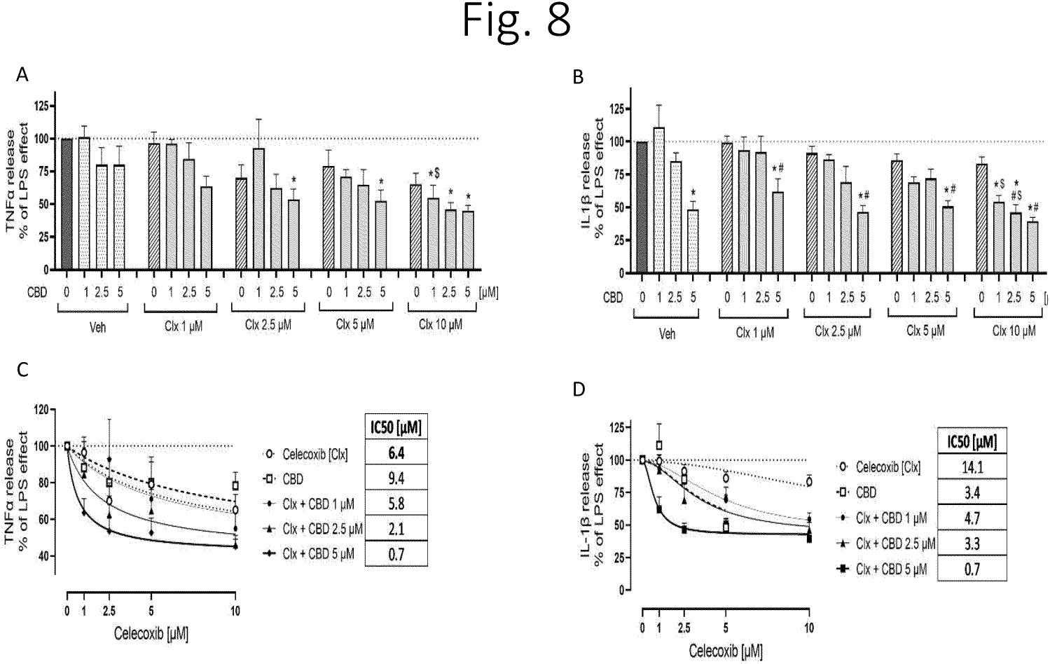

Analysis of the effects of celecoxib, CBD and their combinations on LPS-induced TNFα secretion demonstrated a significant difference between the groups in the suppressive effects on TNFα (F_{19,} ₅₅=3.389, p<0.001) (FIG. 8A) and IL-1 (F_{19,} ₄₀ = 8.123) section (FIG. 8B). As shown, celecoxib or CBD by themselves did not produce significant inhibition of either TNFα or IL-1β (except for the higher concentration of CBD (5 µM), which did produce a significant suppression of IL-1β secretion, p<0.001). Combinations of CBD (5 µM) plus celecoxib (at 2.5, 5 or 10 µM for TNFα and at all concentrations for IL-1β), as well as combinations of celecoxib (10 µM) with CBD (1 or 2.5 µM), produced significant TNFα and IL-1β inhibitory effect, compared with cultures activated with LPS and treated with Veh (p<0.05). Furthermore, combinations of celecoxib (10 µM) with CBD (1 µM) produced a significant TNFα-inhibitory effect, compared with the effects of CBD by itself, and combinations of celecoxib (10 µM) plus either CBD (either 1 µM or 2.5 µM) produced significant IL-1β inhibitory effects, compared with the effects of these concentration of CBD applied by itself (p<0.05).

For a more global analysis of these findings, XY plots were prepared, where the concentrations of celecoxib are plotted on the x-axis and the responses to this drug in combination with various concentrations of CBD, expressed as % of maximal LPS effect, are plotted on the y-axis. Based on these plots, the IC50 of celecoxib and CBD, by themselves or in combination, was computed. The IC50, i.e., the concentrations of celecoxib and CBD, by themselves or in combination, where the response to LPS is reduced by half of the maximal effect, is presented in the Table on the right of each XY plot. As shown, the IC50 of celecoxib by itself for both TNFα (FIG. 8C) and IL-1β (FIG. 8D) was markedly reduced (i.e., the effect of celecoxib was potentiated) when CBD was added together with various concentrations of celecoxib.

These findings demonstrate the synergistic effects of CBD and celecoxib on microglia activation, indicating that CBD can induce a dose-sparing effect, allowing to reduce the celecoxib dose when microglia suppression is required.

### Example 8

### Synergistic Effects of CBD and Celecoxib on α-Synuclein-Induced TNFα Production by BV2 Microglia Cell Cultures

Neuroinflammation, driven by activated microglia and their inflammatory mediators (cytokines), is one of the leading causes of Parkinson’s disease (PD) and other neurodegenerative disorders. Excessive cytokine levels released by microglia in response to e.g., misfolded or aggregated proteins (such as α-synuclein aggregates and/or fibrils), accelerate the DA neuron damage, leading to the motor and cognitive deterioration in PD. Inhibition of COX-2 by celecoxib was shown to decrease microglia activation, and by this way to slow down the degeneration of DA neurons and to improve motor and cognitive scores in animal models of PD. CBD has been shown to exhibit potent neuroprotective, antioxidant and anti-neuroinflammatory properties. Consistently, in animal PD models there is some evidence that CBD can slow down the PD-like symptom progression and severity. In the current Example we show that specific combinations of celecoxib plus CBD are superior to each of these compounds in reducing α-synuclein-induced microglia stimulation.

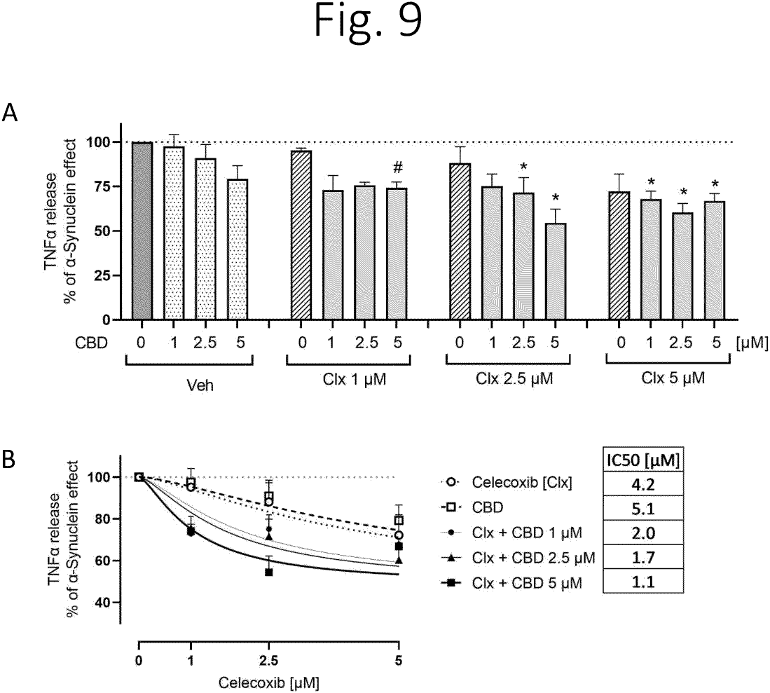

Analysis of the effects of celecoxib, CBD and their combinations on TNFα secretion, induced by exposure of BV2 microglial cells to α-synuclein aggregates, demonstrated a significant difference between the groups (F_{15,32}=4.307, p<0.001; FIG. 9A). Post hoc analysis revealed the following: (1) neither CBD (1, 2.5, or 5 µM) nor celecoxib (1 or 2.5 µM) by themselves affected the release of TNFα by microglia incubated with α-synuclein aggregates/fibrils, (2) combinations of celecoxib (2.5 µM) plus CBD (either 2.5 or 5 µM) significantly decreased the release of TNFα protein from the activated cells (p<0.05 and 0.01, respectively). Celecoxib (5 µM) alone decreased significantly the levels of TNFα released in response to α-Synuclein and the effect was maintained (and was slightly more potent) when Celecoxib at this dose was combined with CBD (at 1, 2.5, or 5 µM) (p<0.05, p<0.01 and p<0.05, respectively). Furthermore, comparisons of the effects of each combination with its components given by themselves revealed that celecoxib (1 µM) plus CBD (5 µM) was significantly more efficient in inhibiting TNFα than celecoxib given alone in the same dose (p<0.05).

For a more global analysis of these findings, an XY plot was prepared, where the celecoxib concentrations are plotted on the x-axis and the response, i.e., release of TNFα (expressed as % of maximal effect) induced by α-synuclein aggregates/fibrils, is plotted on the y-axis. The XY plot shows nonlinear regression curves fitted based on wide-range concentration-response experiments of CBD and celecoxib tested individually, or when celecoxib (1 or 2.5 or 5 µM) was added to various concentrations of CBD (FIG. 9B). Calculation of the Inhibitory Concentration (IC) 50 values for CBD, celecoxib and their combinations are presented in the Table adjacent to the XY plot, demonstrating that the IC50s for celecoxib and CBD by themselves were 4.2 µM and 5.1 µM.Addition of 1 or 2.5 or 5 µM of CBD to each of the tested doses of CBD (1, 2.5 and 5 µM) decreased the celecoxib IC50 to 2.0 µM, 1.7 µM and 1.1 µM, respectively.

The findings demonstrate the synergistic effects of CBD and celecoxib in a model of synucleinopathy-associated neuroinflammation, suggesting that a combination of the two drugs can produce effective, celecoxib dose-sparing effects in PD.

### Example 9

### Attenuation of LPS-Induced Depressive-Like Symptoms by Specific Cannabinoids, Terpenes, Flavonoids, and their Combinations in the LPS-Induced Model of Depression

An LPS model of depression was developed in mice more than two decades ago and was later validated in humans. Since its invention, this model served as one of the most popular and efficient paradigms for assessing inflammatory/microglia-related behavioral/psychiatric disturbances and was utilized in hundreds of studies. LPS-induced depression has been shown to depend on the induction of inflammation and microglia activation, as proved by the amelioration of the depressive symptoms by anti-inflammatory drugs and by microglia suppressive drugs. According to the present invention, the LPS-induced depression model is used to discover novel formulations of cannabinoid(s), with terpene(s) and/or flavonoid(s) suitable for treatment of inflammation-associated depression.

In a preliminary screening experiment, subjects were 4-6 months-old male C57BL/6 (Jackson Laboratories, USA). Animals were housed 2-3/cage, kept in an air-conditioned room (23+°C) and given ad libitum access to food and water. The mice were kept in a reversed light/dark cycle, with lights off from 7 a.m. to 7 p.m. All experiments were approved by the Hebrew University of Jerusalem Ethics Committee on Animal Care and Use. In preparation for the Sucrose Preference Test, mice were habituated 2-3 times to sucrose drinking, by exposing them overnight to two graduated tubes, one containing water and the other containing sucrose solution (1%, w/v). Water and sucrose consumption were measured, and sucrose preference was computed by dividing the amount of sucrose consumed by drinking (ml sucrose) by the total drinking volume (ml of sucrose + water). One day later, the weight of the mice and the food in their cages was weighed for later analysis of food consumption and body weight changes. Groups of mice were injected intraperitoneally (i.p.) with either vehicle (cremophor, ethanol and PBS, 1:1:18 (v/v/v)) or a cannabinoid-based formulation (by itself or with another cannabinoid, or a terpene or a flavonoid). One hour later, each of these groups was injected with LPS (330 µg/kg). All injections were administered at a dose of 10 ml/kg. Four hours following the LPS injection, mice were tested in the Open-Field Test. In this test, the measured locomotor activity reflects not only motor capacity, but particularly the innate motivation for spatial exploration (which increases the distance travelled) and the anxiety level (which reduces the distance travelled, in general, and particularly the distance and time spent in the center of the open field). Each subject was placed near one of the walls of a white 50 X 50 cm² arena with 40 cm high walls. The distance travelled (cm), the velocity of the movement (cm/second), the time spent (seconds), distance travelled (cm) in the center area of the arena, as well as the number of entries from the perimeter area into the center of the arena were automatically measured over a 4-minute period, using EthoVision XT video tracking system and software (Noldus, The Netherlands). Twenty-four hours after the LPS injection, mice were tested in the Social Exploration Test. In this test, the subject was placed in an observation cage containing a small metal wire mesh cage with a conspecific mouse inside it. Social exploration, defined as the time of near contact between the nose of the subject and the juvenile in the small cage was recorded for 4 min, using computerized in-house software. Sucrose preference was assessed, as described above, between 8 to 27 hr post LPS injection. Body weight and weight of the food were measured two hours before the LPS injection, and again after 24 hours, and the change in body weight and food consumption were calculated.

This particular study was conducted in several experimental cohorts, and not all compounds and all measures were studied in all cohort. However, each cohort included a vehicle-treated group; therefore, the scores in each behavioral test were converted to % of mean vehicle-treated group in every individual cohort/experiment. Statistical comparisons were computed using SPSS software and consisted of one-way analyses of variance (ANOVA), followed by post-hoc comparisons with the Student-Newman-Keuls method.

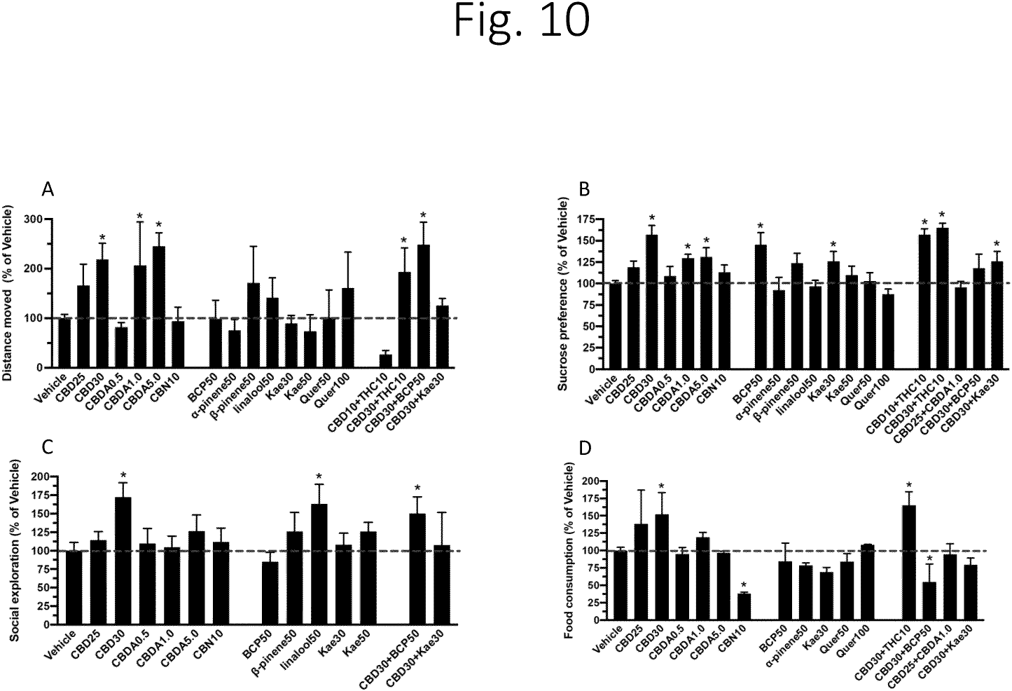

In the open field test, there was a significant difference between the groups in the distance travelled (F_{17,149}=2.767, p<0.001). Groups treated with CBD (30 mg/kg), CBDA (1 or 5 mg/kg), CBD (30 mg/kg) plus THC (10 mg/kg), and CBD (30 mg/kg) plus BCP (50 mg/kg) displayed significantly increased distance travelled in the open field compared with vehicle-treated groups (FIG. 10A). In the sucrose preference test, there was a significant difference between the groups (F_{18,165}=5.911, p<0.0001). (p<0.0001). Post-hoc tests revealed that groups treated with CBD (30 mg/kg), CBDA (either 1 or 5 mg/kg), β-caryophyllene (BCP, 50 mg/kg), Kaempferol (Kae) (30 mg/kg), CBD (30 mg/kg) plus THC (10 mg/kg), and CBD (30 mg/kg) plus kaempferol (30 mg/kg) displayed significantly increased sucrose preference compared with vehicle-treated groups (FIG. 10B). In the social exploration test, there was a significant difference between the groups (F_{13,105}=2.274, p<0.05). Groups treated with CBD (30 mg/kg), linalool (50 mg/kg) and CBD (30 mg/kg) plus BCP (50 mg/kg), displayed significantly increased sucrose preference compared with vehicle-treated groups (FIG. 10C). There was a significant difference between the groups in the amount of food consumption over 24 hr (F₁₅,₁₅₁=3.357, p<0.0001). Groups treated with CBD (30 mg/kg) or with CBD (30 mg/kg) plus THC (10 mg/kg), consumed significantly more food than the vehicle-treated group, whereas groups treated with cannabinol (10 mg/kg) or CBD (30 mg) plus BCP (50 mg) consumed significantly less food than vehicle-treated group (FIG. 10D).

### Example 10

### Effects of Acute CBD Administration on LPS-Induced Depressive-Like Behavior in Adult and Young-Adult Male Mice

CBD is by far the most studied cannabinoid in terms of its effects on depressive-like symptomatology in animal models of depression. Still, there are gaps in our knowledge regarding the age- and sex-related differential effects of various regimens of CBD administration. In the following Examples we examined the effects of these parameters on the behavioral responsiveness to CBD in LPS-treated mice, using adult (6-9-month-old) and young (2.5-3 months-old) male and female animals.

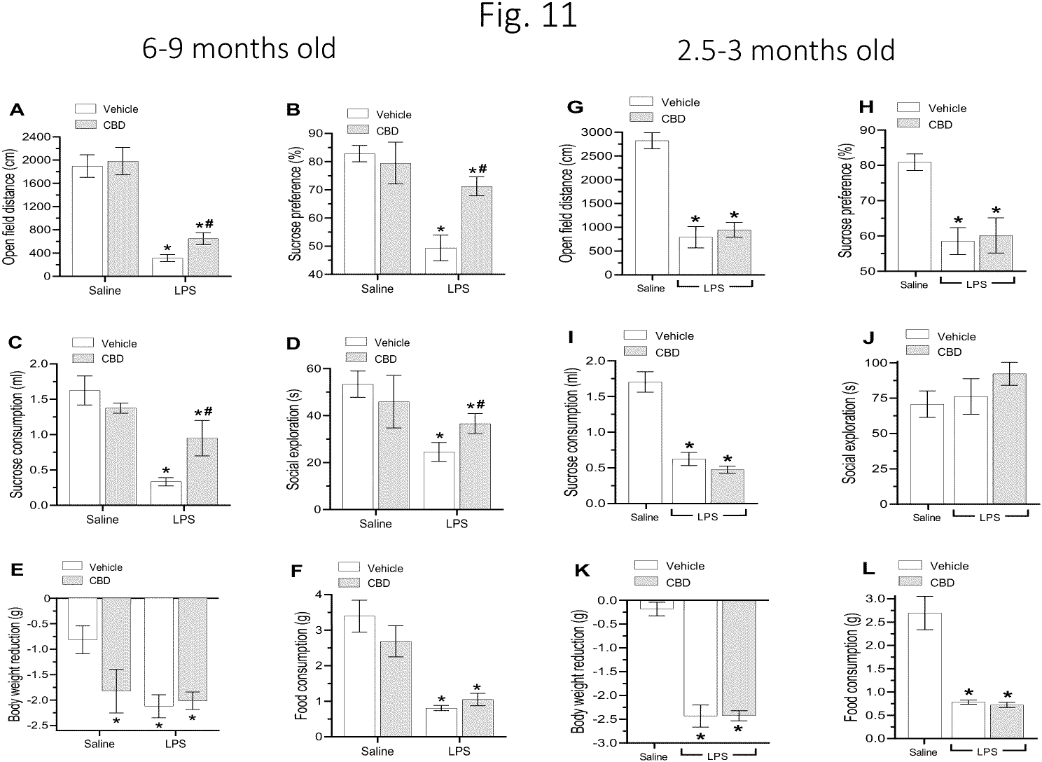

In adult male mice, there was a main effect of LPS on the distance traveled in the open field test (F_{1,43}=106.506, p<0.001) (FIG. 11A). Post-hoc analysis revealed that the LPS-injected groups were significantly different from their respective saline-injected controls (*p<0.001). Furthermore, among the LPS-injected groups, CBD (30 mg/kg) significantly attenuated the suppressive effect of LPS on this measure (^{#}p<0.05). In the sucrose preference test, there was a main effect of LPS (F_{1,43}=106.506, p<0.001). CBD-treated mice, but not vehicle-treated mice, showed a reduction in the negative effect of LPS on sucrose preference, as reflected by a significant CBD by LPS interaction (F_{1,34}=4.576, p<0.05) (FIG. 11B). Post-hoc analysis revealed a significant difference between saline- and LPS-injected vehicle-treated mice (*p<0.001), as well as between CBD- and vehicle-treated LPS-injected groups (#p<0.001). In the sucrose consumption measure, there was a main effect of LPS (F_{1,36}=12.585, p<0.005) (FIG. 11C). Post-hoc analysis revealed that the LPS-injected groups were significantly different then their respective saline-injected controls (*p<0.001). Additionally, among the LPS-injected groups, there was a significant difference between the CBD- and vehicle-treated group (^{#}p<0.05). In the social exploration test, LPS-injected mice showed a significant reduction in the amount of time spent exploring the juvenile conspecific, as reflected by a main effect of LPS on this measure (F_{1,44}=8.733, p<0.05) (FIG. 11D). Post-hoc analysis revealed a significant difference between saline and LPS-injected vehicle-treated mice (*p<0.005). Additionally, among the LPS-injected groups, there was a significant difference between the CBD- and vehicle-treated groups (^{#}p<0.05). LPS significantly reduced body weight, as reflected by a main effect of LPS on this measure (F_{1,47}=5.873, p<0.05) (FIG. 11E). Post-hoc analysis revealed a significant difference between saline-injected vehicle-treated mice and LPS-injected mice (*p<0.005). Food consumption was significantly reduced by LPS, as reflected by a main effect of LPS on this measure (F_{1,47}=70.046, p<0.001) (FIG. 11F). Post-hoc analysis revealed that the LPS-injected groups were significantly different then their respective saline-injected controls (*p<0.001).

In young-adult male mice, there were significant differences between the experiment groups in the distance moved in the open field test (F2,40=32.742, p<0.001) (FIG. 11G). Post-hoc analysis revealed that the LPS-injected groups were significantly reduced in each of these measures when compared to their saline-injected controls (p<0.001*). In the sucrose preference test, there were significant differences between the experiment groups in sucrose preference (FIG. 11H) and sucrose consumption (FIG. 11I) (F_{2,40}=6.285, p<0.005 and F_{2,40}=52.335, p<0.001, respectively). Post-hoc analysis revealed that the LPS-injected groups were significantly reduced in each of these measures when compared to their saline-injected controls (p<0.005*). Social exploration time was not significantly different between the experiment groups (FIG. 11J). Body weight reduction (FIG. 11K) and food consumption (FIG. 11L) were significantly different between the experiment groups (F_{2,40}=60.642, p<0.001 and F_{2,40}=50.538, p<0.001). Post-hoc analysis revealed that the LPS-injected groups displayed significant reductions in each of these measures when compared to their saline-injected controls (p<0.005*).

These findings indicate that in a model of inflammation-induced depression, CBD produces anti-depressant and anxiolytic effects in adult, but not young-adult mice.

### Example 11

### Effects of Repeated (Prophylactic) CBD Administration on LPS-Induced Depressive-Like Behavior in Young Male Mice

Given that acute CBD administration did not reverse the LPS-induced depressive-like behavior in young-adult mice, the effects of semi-chronic (prophylactic) CBD administration were examined in young-adult (3.5-month-old) mice to determine if it produces therapeutic effects. Mice were injected (i.p.) with either vehicle or CBD (30 mg/kg) for three consecutive days. 1 hr after the last injection, they were injected with either saline or LPS (330 µg/kg), and were then examined according to the methods described in Example 8 and 9.

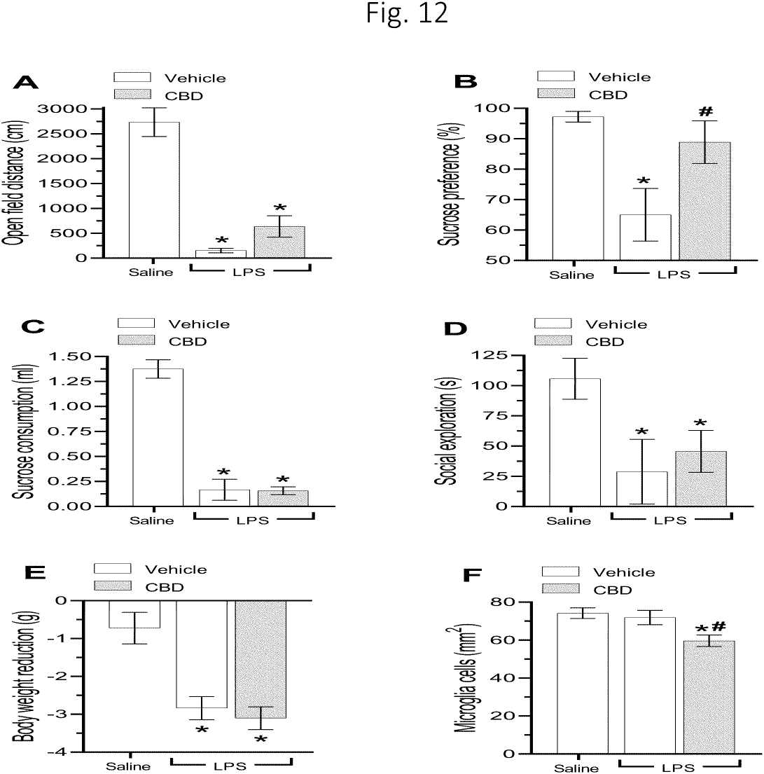

(FIG. 12A) In the open field test, there was a significant difference between the experiment groups in distance measurement (F_{2,16}=34.65, p<0.0001). Post-hoc analysis revealed that the LPS-injected groups displayed significantly lower activity compared to their saline-injected controls (p<0.0001*). (FIG. 12B) In the sucrose preference tests, there was a significant difference between the groups (F_{2,13}=6.753, p<0.01). Post-hoc analysis revealed a significant suppression of sucrose preference in the LPS-injected group treated with Vehicle (p<0.005*), which was completely reversed by CBD treatment (p<0.05^{#}). (FIG. 12C) There was a significant difference between the experiment groups in sucrose consumption (F₂,₁₇=78.66, p<0.0001). Post hoc analysis revealed a significant difference in this measure between the Sal-injected group and both LPS-injected groups (p<0.0001*). (FIG. 12D) In social exploration test, there was a significant difference between the experiment groups (F2,16 =3.823, p<0.05). Post-hoc analysis revealed a significant difference in this measure between the Sal-injected group and both LPS-injected groups (p<0.05*). (FIG. 12E) There was a significant difference between the experiment groups in body weight reduction (F_{2,17}=14.19, p<0.0005). Post-hoc analysis revealed a significant difference in this measure between Sal-injected group and LPS-injected groups (p<0.001*). (FIG. 12F) There was a significant difference between the experiment groups in hippocampus microglia number (F_{2,153}=6.478, p<0.005). Post-hoc analysis revealed that among LPS-treated mice, the microglia density was significantly reduced in CBD- as compared with Veh-treated mice (p<0.001*).

These findings indicate that in contrast with acute CBD administration, which had no effects in young mice, repeated, prophylactic administration of CBD in young mice reverses LPS-induced anhedonia and reduces hippocampal microglia density, although some LPS-induced behavioral symptoms are still unaffected by this treatment.

### Example 12

### Effects of an Oral CBD Formulation Based on Pro-Nano Lipospheres on LPS-Induced Depressive-Like Effects

The experiments in all of the Examples of this patent are conducted using i.p. injections of the active treatment compounds. To pursue the translational potential of the treatments described herein, we examined the effects of CBD dissolved in a self-emulsifying drug delivery system termed Pro-nano lipospheres (PNL), which is a mixture of a lipid, surfactants and co-solvent that dissolve the lipophilic molecules. In solution, this formulation allows the formation of a thermodynamically stable oil/water nano dispersion with nano particles of 50 nm or less. Particles entrap the lipophilic CBD molecules in their lipid core, and thus increase solubility and enhance oral bioavailability of CBD.

The PNL solution was prepared from lecithin and ethyl lactate, heated to 37° C. until completely dissolved, followed by sequential addition of Tricaprin, Tween® 20, Span® 80 and Kolliphor® RH40. The mixture was heated to 40° C. until a homogenous, clear solution was formed. On each experimental day, the PNL solution was freshly prepared by dispersion of the formulation in pre-heated water (1:9 v/v) and vortexing the mixture for 30 seconds. The solution was poured into graduated drinking tubes, allowing free consumption by the mice.

The experimental design included four groups, pre-treated with the PNL solution for 4 days, after which one of the groups weas injected with saline and 3 groups were injected with LPS. The saline group and one of the LPS-injected group were treated with the PNL solution without CBD, while two LPS-groups were treated with one of two concentrations of CBD (plant-derived with 99% purity, BOL, Israel). The CBD was added to the PNL solution at two concentrations: 75 or 150 mg/ml solution. Considering that mice in the two groups drank an average of 2.5 and 2.1 ml/day, these concentrations yielded consumption of 47.1 and 78.2 mg/kg per day in the two groups, respectively. Four hours following the LPS injection, the animals were tested in the open-field test, and at 10-24 hr post-injection the sucrose preference test was conducted.

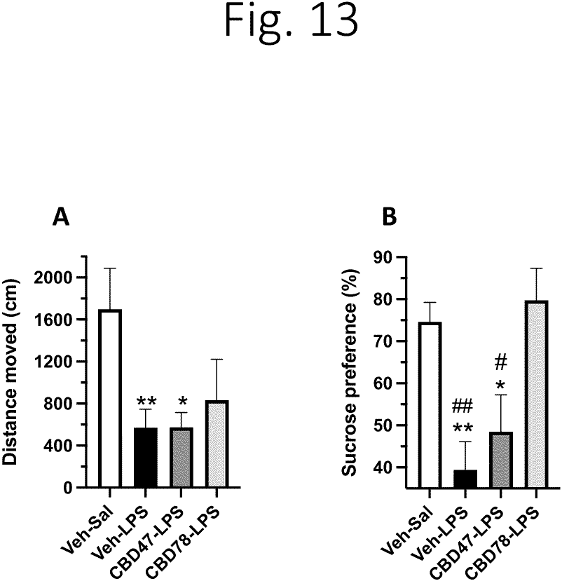

The results show a significant difference between the groups in open-field activity (F₃,₁₄=3.52, 0<0.05) (FIG. 13A). Post-hoc tests revealed that mice treated with either PNL solution without CBD or with CBD solution at 47.1 mg/kg showed a significant reduction in the distance moved in the open field, compared with saline-injected mice. In contrast, no such difference was found with respect to mice treated with the 78 mg/kg solution. In the sucrose preference test, there was a significant difference between the groups (F_{3,14}=7.93, 0<0.005) (FIG. 13B). Post-hoc tests demonstrated that mice treated with PNL solution by itself or with 47.1 mg/kg CBD displayed a significant reduction in sucrose preference, compared with saline-treated mice (p<0.01 and p<0.05, respectively). CBD at 78 mg/kg completely reversed this effect, i.e., mice in this group displayed significantly elevated preference compared with mice treated with either PNL solution without CBD or with CBD solution at 47.1 mg/kg (p<0.01 and p<0.05, respectively).

These preliminary findings suggest that similarly to the findings of Example 11, repeated prophylactic administration of CBD, using a PNL-based oral formulation, reverses the anhedonic effect of LPS, while not influencing the LPS-induced locomotor/anxiogenic effect in the Open-Field test.

### Example 13

### Effects of Acute CBD Administration on LPS-Induced Depressive-Like Behavior in Adult and Young-Adult Female Mice

Given that sex is known to influence the responsiveness to CBD on several physiological parameters, in the current study we assessed the effects of CBD on LPS-induced behavioral changes in adult (6-9 months-old) or young-adult (2.5-3 months-old) female mice.

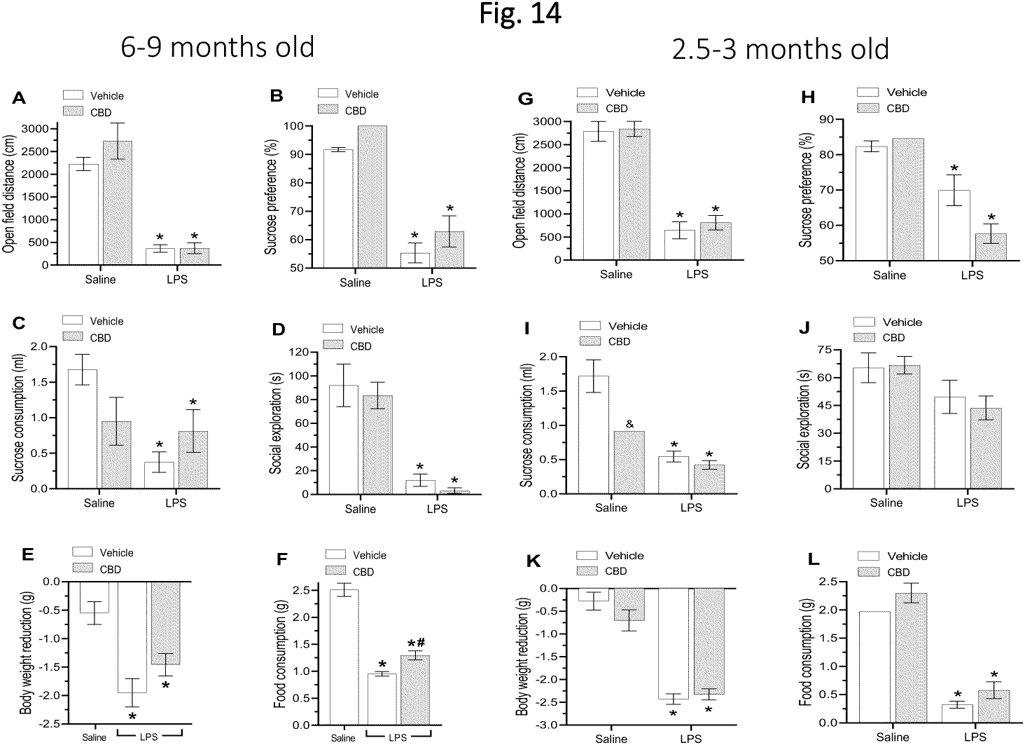

In the open field test, LPS-injected adult female mice showed a significant reduction in the distance travelled, as reflected by main effects of LPS on this measure (F_{1,24}=66.252, p<0.001) (FIG. 14A). Post-hoc analysis revealed that the LPS-injected groups were significantly different then their saline-injected controls (*p<0.001). In the sucrose preference test, there were significant differences between the experiment groups in sucrose preference (FIG. 14B) and sucrose consumption (FIG. 14C) (F_{1,16}=28.200, p<0.001 and F_{1,20}=6.081, p<0.05, respectively). Post-hoc analysis revealed that the LPS-injected groups were significantly reduced in each of these measures when compared to their saline-injected controls (p<0.001* and **p<0.005, respectively). In the social exploration test, LPS-injected mice showed a significant reduction in the amount of time spent exploring the juvenile mice, as reflected by a main effect of LPS on this measure (F_{1,12},=39.015, p<0.001) (FIG. 14D). Post-hoc analysis revealed that the LPS-injected groups were significantly different then their respective saline-injected controls (*p<0.005). Body weight reduction was significantly different between the experiment groups (F_{2,9}=10.844, p<0.005) (FIG. 14E). Post-hoc analysis revealed that the LPS-injected groups displayed significant reductions in body weight when compared to their saline-injected controls (p<0.001*). Food consumption was significantly different between the experiment groups (F_{2,9}=85.662, p<0.001) (FIG. 14F). Post-hoc analysis revealed that the LPS-injected groups displayed significant reductions in food consumption when compared to their saline-injected controls (p<0.001*). Furthermore, among the LPS-injected groups, there was a significant difference between the CBD- and vehicle-treated group (#p<0.05).

In young-adult female mice, LPS-injection produced a significant reduction in the distance travelled in the open-field test, as reflected by main effect of LPS on this measure (F₁₃₅=123.643, p<0.001) (FIG. 14G). Post-hoc analysis revealed that the LPS-injected groups were significantly different then their saline-injected controls (*p<0.001). In the sucrose preference test, LPS-injected mice showed a significant reduction in preference, as reflected by main effect of LPS on this measure (F_{1,31}=13.771, p<0.005) (FIG. 14H). Post-hoc analysis revealed that the LPS-injected groups were significantly different then their saline-injected controls (*p<0.001). Sucrose consumption was significantly reduced in LPS-injected mice and in CBD-treated mice, as reflected by the main effects of LPS (F_{1,31}=30.823, p<0.001) and CBD (F_{1,31}=30.823, p<0.005) on this measure (FIG. 14I). Post-hoc analysis revealed that the LPS-injected groups were significantly different then their saline-injected controls (*p<0.001). In addition, among saline-injected mice, there was a significant difference between vehicle- and CBD-treated mice (**p<0.005). In the social exploration test, LPS reduced the exploration time in both vehicle and CBD-treated groups, but these results did not reach statistical significance (FIG. 14J). LPS significantly reduced body weight (FIG. 14K) and food consumption (FIG. 14L), as reflected by main effects of LPS on each of these measures (F₁,₃₅=139.338, p<0.001 and F_{1,16}= 88.995, p<0.001, respectively). For each of these measures, post-hoc analysis revealed a significant difference between saline- and LPS-injected mice (*p<0.001).

These findings indicate that in the LPS-model of depression, acute administration of CBD has no beneficial therapeutic effects in either adult or young-adult female mice.

### Example 14

### Effects of CBD, Kaempferol and Their Combination on LPS-Induced Depressive-Like Behavior in Adult Female Mice

Kaempferol is one of the major flavonoids found in many cannabis strains. Furthermore, kaempferol is a major component of plants other than cannabis, such as Lavender, Hypericum perforatum (St. John’s Wort), Saffron, wild ginger, lauraceae, roseroot and ginkgo biloba results presented in Example 4, in the present Example we examined the possible synergistic effects of kaempferol and CBD on LPS-induced depressive/anxiety-like symptoms.

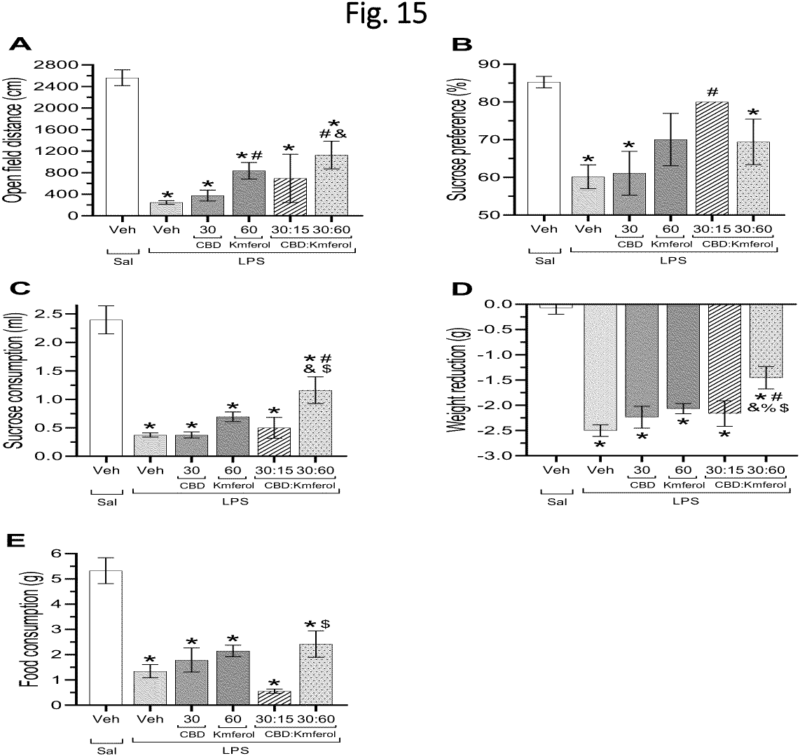

In the open field test, there was a significant difference between the experiment groups in distance traveled (F_{2,16}=34.65, p<0.0001) (FIG. 15A). Post-hoc analysis revealed that this parameter was significantly reduced in all of the LPS-injected groups, compared with saline-injected controls (p<0.0001). Among the LPS-injected groups, the groups treated with kaempferol (60 mg/kg), by itself or together with CBD (30 mg/kg), displayed a significant elevation in open-field activity, compared with the Veh-treated group (p<0.001*). Furthermore, the group treated with the combination of CBD (30 mg) plus kaempferol (60 mg) displayed significantly higher open-field activity than the group treated with CBD by itself (p<0.005^{#}). Sucrose preference was significantly different between the groups (F_{5,55}=3.287, p<0.05) (FIG. 15B). Post-hoc analysis revealed that the LPS-injected groups treated with Veh, CBD or the combination of CBD (30 mg) plus kaempferol (60 mg) displayed significantly reduced sucrose preference compared with the saline-injected control group (p<0.01, p<0.01 and p<0.05 respectively), whereas the group treated with the combination of CBD (30 mg) plus kaempferol (15 mg) displayed significantly higher preference compared with the Veh-treated group (p<0.05*). Sucrose consumption was significantly different between the groups (F5,59=20.66, p<0.0001) (FIG. 15C). Post-hoc analysis revealed that all of the LPS-injected groups displayed significantly reduced consumption, compared with the saline-injected control group (p<0.0001). Among the LPS-injected groups, treatment with the combination of CBD (30 mg) plus kaempferol (60 mg/kg) resulted in significantly increased sucrose consumption, compared with groups treated with Veh (p<0.005*), CBD (30 mg/kg) (p<0.005^{#}), and the combination of CBD (30 mg) plus kaempferol (60 mg/kg) (p<0.05^{$}). Post-injection changes in body weight were significantly different between the groups (F_{5,59}=25.38, p<0.0001) (FIG. 15D). Post-hoc analysis revealed that all of the LPS-injected groups displayed significant body-weight loss, compared with the saline-injected control group (p<0.0001). Among LPS-injected groups, the group treated with the combination of CBD (30 mg) plus kaempferol (60 mg/kg) displayed significantly smaller weight loss when compared with groups treated with Veh (p<0.0001*), CBD (30 mg/kg) (p<0.005^{#}), kaempferol (60 mg/kg) (p<0.05^{&}) and combination of CBD (30 mg) plus kaempferol (15 mg) (p<0.05^{$}). Food consumption was significantly different between the experiment groups (F_{5,59}=13.00, p<0.0001) (FIG. 15E). Post-hoc analysis revealed that the all of the LPS-inj ected groups displayed significant reductions in food consumption, compared with the saline-injected control group (p<0.0001). Among LPS-injected groups, the group treated with the combination of CBD (30 mg) plus kaempferol (60 mg/kg) displayed significantly greater food consumption than the group treated with the combination of CBD (30 mg) plus kaempferol (15 mg) (p<0.05^{$}).

These findings indicate that in adult females, CBD and kaempferol have synergistic therapeutic effects on inflammation-induced depression- and motor/anxiety-like symptoms.

### Example 15

### Effects of CBDA on LPS-Induced Depressive-Like Behavioral Symptoms in Male and Female Mice

Given the mixed effects that CBD administration had in the reversal of LPS-induced depressive-like behavior in young-adult mice, the effects of CBDA by itself, and in combination with CBD, were examined in young-adult (2.5-3.5 months old) female and male mice. Mice were injected (i.p.) with either vehicle, CBDA or CBD and CBDA combination, and 1 hr later with LPS (330 µg/kg). Following injections, the mice were examined according to the methods described in Example 8, apart from the Porsolt forced swim test that replaced the open field test in male mice. In this test, mice were placed in a plastic cylinder (with a height of 20 cm and diameter of 50 cm), filled with 25° C. water. The time spent immobile, defined as the absence of all movement except for motions required to maintain head above water, and the latency for the first immobility episode, were recorded for 6 min, and manually analyzed by an experienced experimenter.

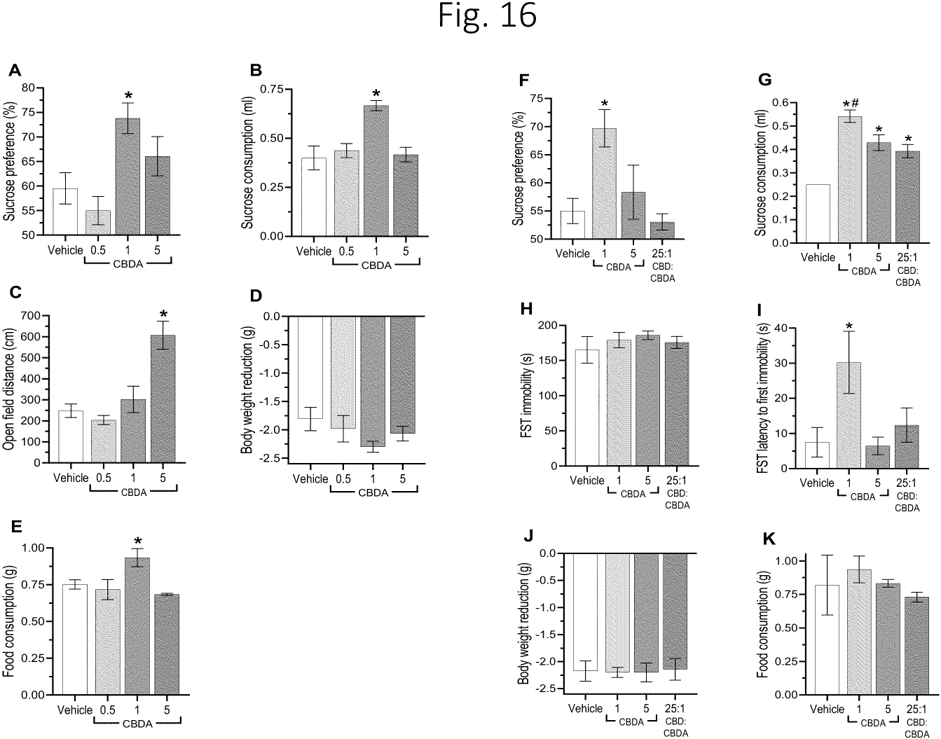

In young-adult female mice, there was a significant difference between the experiment groups in sucrose preference (F_{3,17}=5.363, p<0.01) (FIG. 16A). Post-hoc analysis revealed that CBDA (1 mg/kg) treatment group was significantly increased in this measurement when compared to groups treated with Vehicle (p<0.01*) and CBDA (0.5 mg/kg) (p<0.005*). Sucrose consumption was significantly different between the experiment groups (F₃,₁₇=10.03, p<0.0005) (FIG. 16B). Post-hoc analysis revealed that CBDA (1 mg/kg) treatment group was significantly increased in this measurement when compared to groups treated with Vehicle (p<0.0005*) CBDA (0.5 mg/kg) (p<0.005*) and CBDA (5 mg/kg) (p<0.0005*). In the open field test, there was a significant difference between the experiment groups in distance measurement (F3, 18 = 14.21, p<0.0001) (FIG. 16C). Post-hoc analysis revealed that CBDA (5 mg/kg) treatment group was significantly increased in this measurement when compared to groups treated with Vehicle (p<0.0001*) CBDA (0.5 mg/kg) (p<0.0001*) and CBDA (1 mg/kg) (p<0.0005*). There was no significant difference between the experiment groups in body weight reduction (FIG. 16D). Food consumption was significantly different between the experiment groups (F3, 19 = 5.099, p<0.01) (FIG. 16E). Post-hoc analysis revealed that CBDA (1 mg/kg) treatment group was significantly increased in this measurement when compared to groups treated with Vehicle (p<0.05*) CBDA (0.5 mg/kg) (p<0.01*) and CBDA (5 mg/kg) (p<0.005*).

In young-adult male mice, there was a significant difference between the experiment groups in sucrose preference (F3, 19 = 7.708, p<0.005) (FIG. 16F). Post-hoc analysis revealed that CBDA (1 mg/kg) treatment group was significantly increased in this measurement when compared to groups treated with Vehicle (p<0.005*), CBDA (5 mg/kg) (p<0.05*) and combination of CBD (25 mg/kg) and CBDA (1 mg/kg) (p<0.0005*). Sucrose consumption was significantly different between the experiment groups (F3, 22 = 18.65, p<0.0001) (FIG. 16G). Post-hoc analysis revealed that CBDA (1 mg/kg) treatment group was significantly increased in this measurement when compared to groups treated with Vehicle (p<0.0001*), CBDA (5 mg/kg) (p<0.01*) and combination of CBD (25 mg/kg) and CBDA (1 mg/kg) (p<0.001*). In addition, there was a significant increase in this measurement in groups that were treated with CBDA (5 mg/kg) (p<0.005^{#}) and with combination of CBD (25 mg/kg) and CBDA (1 mg/kg) (p<0.005^{&}). In the forced swim test, there was no significant difference between in the experiment groups in time spent in immobility (FIG. 16H). There was a significant difference in the latency to the first episode of immobility between the experiment groups F_{3,20}=4.039, p<0.05) (FIG. 16I). Post-hoc analysis revealed that CBDA (1 mg/kg) treatment group was significantly increased in this measurement when compared to groups treated with Vehicle (p<0.01*), CBDA (5 mg/kg) (p<0.005*) and combination of CBD (25 mg/kg) and CBDA (1 mg/kg) (p<0.05*). There were no significant differences between the experiment groups in body weight reduction and food consumption (FIGS. 16J-16K).

These findings indicate that in young-adult female and male mice (which do not respond to CBD), acute administration of CBDA produces dose-dependent beneficial effects on inflammation-induced behavioral abnormalities. A combination of an effective CBDA dose with CBD abrogated the effect of CBDA, indicating that the inefficacy of such a combination in inflammation-associated depression.

### Example 16

### Effects of CBDA on LPS-Induced Microglial Alterations in Male and Female Mice

Given the suppressive effect of CBD on hippocampal microglia numbers in LPS-injected mice, the effect of Cannabidiolic acid (CBDA), as well as a combination of CBD with the flavonoid kaempferol, was examined to determine if they also produce a similar effect. Mice were firstly injected (i.p.) with CBDA or CBD/kaempferol combination, and 1 hr later were injected with LPS (300 mg/kg in 10 ml/kg saline). Brains were removed 24 hr later and prepared for immunohistochemistry, using the microglia marker Iba-1.

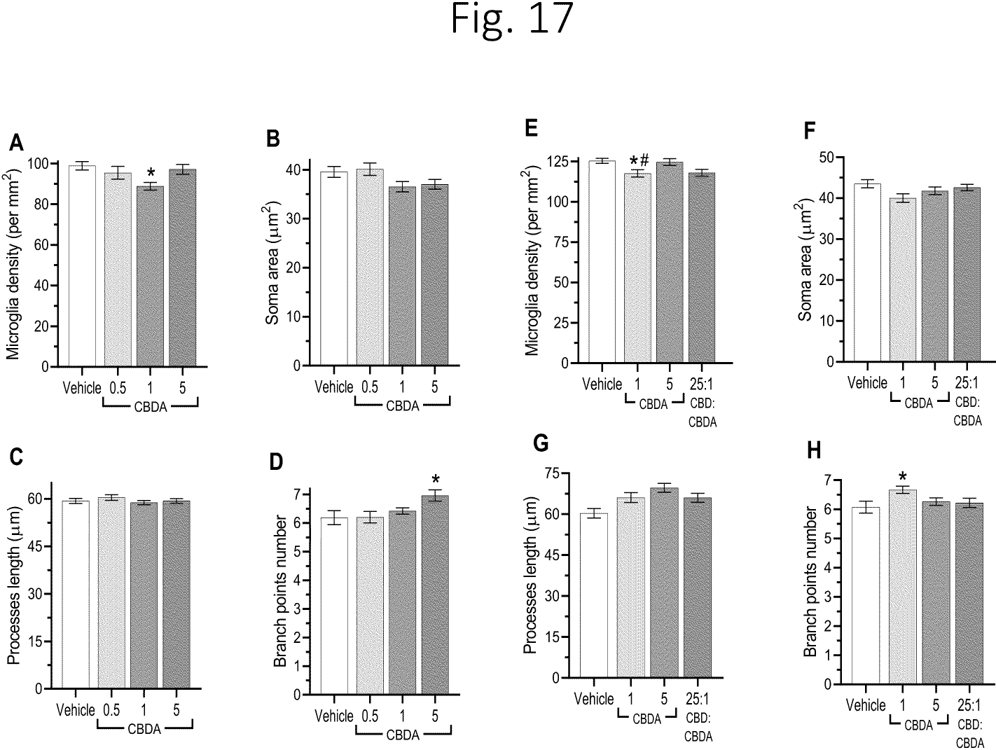

In young-adult female mice, hippocampal microglia density was significantly different between the experiment groups (F_{3,100}=3.792, p<0.05) (FIG. 17A). Post-hoc analysis revealed that CBDA (1 mg/kg) treatment group was significantly reduced in this measurement when compared to groups treated with Vehicle (p<0.005*) and CBDA (5 mg/kg) (p<0.05*). There was no significant difference between the experiment groups in microglia soma area and processes length (FIGS. 17B-17C). Number of branch points of microglia processes was significantly different between the experiment groups (F₃, ₁₀₁=3.762, p<0.05) (FIG. 17D). Post-hoc analysis revealed that CBDA (5 mg/kg) treatment group was significantly increased in this measurement when compared to groups treated with Vehicle (p<0.005*), CBDA (0.5 mg/kg) (p<0.01*) and CBDA (1 mg/kg) (p<0.05*).

In young-adult male mice, hippocampal microglia density was significantly different between the experiment groups (F_{3,179}=3.844, p<0.05) (FIG. 17E). Post-hoc analysis revealed that CBDA (1 mg/kg) treatment group was significantly reduced in this measurement when compared to groups treated with Vehicle (p<0.05*) and CBDA (5 mg/kg) (p<0.05*). In addition, combination of CBD (25 mg/kg) and CBDA (1 mg/kg) was reduced when compared to Vehicle-treated group (p<0.05^{#}) and to CBDA (5 mg/kg)-treated group (p<0.05^{#}). There was no significant difference between the experiment groups in microglia soma area and processes length (FIGS. 17F-17G). Number of branch points of microglia processes was significantly different between the experiment groups (F_{3,125}=2.761, p<0.05) (FIG. 17H). Post-hoc analysis revealed that CBDA (1 mg/kg) treatment group was significantly increased in this measurement when compared to groups treated with Vehicle (p<0.001*) and with combination of CBD (25 mg/kg) and CBDA (1 mg/kg) (p<0.05*).

These findings indicate that in young adult females and male mice, acute administration of CBDA produces dose-dependent reduction in microglia density and altered morphology, in correspondence with the effects of the treatment on the behavioral symptoms.

### Example 17

### Synergistic Effects of Celecoxib and CBD in the Porsolt Forced-Swim and the Elevated Plus Maze Tests

Over the last decade, it has been shown that inflammatory processes, in general, and activation of brain microglia cells, in particular, are causally involved in the development of depression. Consistently, studies in experimental animal models of depression, as well as randomized clinical trials in depressed patients, demonstrated the antidepressant efficacy of anti-inflammatory drugs, including celecoxib and CBD (each by itself).

The Porsolt forced swim test (FST) measures coping strategy to an acute inescapable stress, which is a major contributing factor for the development of depression in humans. Indeed, drugs that have efficacious antidepressant effect in humans have been shown to promote active coping strategy in the FST. Therefore, the measures of immobility (passive coping) and the latency to exhibit the first immobility bout in the FST are considered as measures of despair and “depression-like” behavioral responses to stress. The Elevated plus maze test (EPM) is the most popular test for assessing anxiety responses in rodents, and it has been validated to assess the anti-anxiety effects of various pharmacological agents.

Subjects were 6-7 months old male and 3.5 months old female C57BL/6 mice. Animals were housed 2-3 per cage in an air-conditioned room (23° C.), with food and water ad libitum, and under light/dark 12/12 illumination (light on from 7 a.m. to 7 p.m). All experiments were approved by the Hebrew University of Jerusalem Ethics Committee on Animal Care and Use. Subjects were randomly assigned to treatment groups. Following body weight measurement, the mice received an intraperitoneal (i.p.) injection (in a volume of 10 mg/kg) of either 10 mg/kg CLX (Sigma, Israel), 5 mg/kg CBD (BOL, Israel), or a combination of 10 plus 5 mg/kg CLX and CBD, respectively. CLX and CBD and were dissolved in ethanol/cremophor/saline solution, at a ratio of 1:1:18. Control mice were injected with the vehicle only. In the Forced Swim Test, mice were individually placed for 6 minutes in a Plexiglass cylinder (with a 20 cm diameter and 40 cm height), containing 15 cm-depth water at 23-25° C., and their behavior was videotaped for this entire duration. The time spent in immobility, defined as the absence of all movement except motions required to maintain the animal’s head above the water, and the latency to first immobility episode, were recorded by an observer. In this experiment, we examined the antidepressant effects of CLX (10 mg/kg) and of CBD (5 mg/kg), by themselves, or in combination (10:5 mg/kg of CLX:CBD). Additional group was treated with vehicle only. 60 minutes following the treatment injection, the mice behavior was examined in the forced swim test. All data are presented as mean ± SEM. Statistical comparisons were computed using SPSS software and consisted of two-way analyses of variance (ANOVA), with the treatment and the sex as between-subjects factors, followed by post hoc analyses with the Student Newman-Keuls procedure.

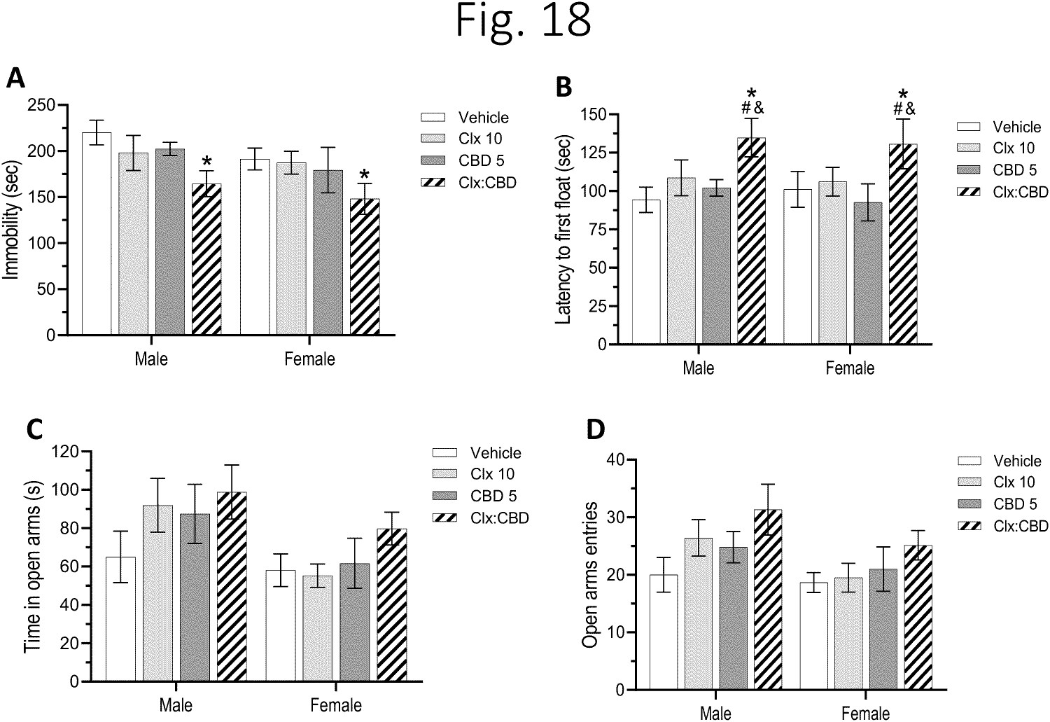

Analysis of the results of the immobility time (in seconds (sec), out of a total of 360 sec) revealed a significant overall difference between the groups (F_{3,70}=4.149, p<0.01), with no effects of sex or treatment by sex interaction (FIG. 18A). Post-hoc analysis revealed that treatment with CLX plus CBD combination significantly reduced the immobility of mice (P<0.05), when compared to mice that were injected with vehicle. Analysis of the results of the latency to the first episode of immobility revealed a significant treatment effect (F_{3,70}=5.118, p<0.01) (FIG. 18B), with no effects of sex or treatment by sex interaction. Post-hoc analysis revealed that treatment with the CLX plus CBD combination significantly increased the latency to first float, as compared with each compound by itself and to the vehicle treatment (P<0.05).

In a separate experiment, we examined the effects of CLX, CBD and their combination on anxiety levels, as reflected by activity in the EPM. All of the methodological aspects are similar to the experiment described above except that instead of the Forced Swim apparatus, mice were place in an Elevated Plus Maze. The apparatus, situated 40 cm above the floor, consists of a plus-like shaped maze with two white plastic closed arms and two opposite white open arms. Each arm is 30 cm long and 6 cm wide. Each mouse was placed invariably in the center of the EPM, with its face toward an open arm. Behavior in the maze was recorded for 4 minutes and coded using EthoVision XT video tracking system and software (Noldus, The Netherlands). The maze was thoroughly cleaned with ethanol and with tap water and dried between subjects in order to eliminate any odor cues. Anxious behavior was measured by the time (in seconds) spent in the open arms, as well as by the number of entries into the open arms.

Analysis of the results revealed a trend for more time in the open arms and the number of entries into the open arms in the group treated by the CLX plus CBD combination vs. the Vehicle-treated group, but these findings did not reach statistical significance. Specifically, for the time in the open arms, there was an effect of sex on the time spent in the open arms of the maze (in seconds (sec), out of 300 sec) (F1, 62=4.651, p<0.05), with males spending more time in the open arms than females. Although there was no overall treatment effect, planned comparison of the effect of the CLX plus CBD combination group vs. the Vehicle treated group showed a clear trend for a differential effect (p=0.057) (FIG. 18C). In the analysis of the results of the entries into the open arms there was a trend for a sex effect (p=0.081), and no treatment effect (p=0.105) (FIG. 18D).

These findings demonstrate a synergistic antidepressant effect of celecoxib and CBD, such that low doses of each compound, which do not produce significant effects by themselves, produce an antidepressant effect in the Forced Swim Stress test. Given that when used clinically, celecoxib produces serious adverse effects, this finding suggests that CBD can produce a dose-sparing effect when used with celecoxib as an antidepressant procedure. A similar finding was obtained with respect to the effects of the celecoxib plus CBD combination on anxiety-related behavior.

### Example 18

### Effects of Celecoxib, CBD and Their Combinations on Inflammation-Induced Behavioral Impairments and Anhedonia

In the current example, we examined the antidepressant and anxiolytic effects of celecoxib in combination with CBD in the LPS-induced model of inflammatory depression. Given that a major aim of this example was to demonstrate a possible celecoxib dose-sparing effect by CBD, we examined the effects of three doses of celecoxib (10, 20 and 30 mg/kg), by themselves or in combination with a dose of CBD (30 mg/kg), which has been previously shown to produce partial antidepressant and anxiolytic effects in animal models.

Subjects were 7 months-old male C57BL/6 mice. Animals were housed 2-3 per cage in an air-conditioned room (23° C.), with food and water ad libitum. They were kept in a reversed light/dark cycle, with lights off from 7 a.m. to 7 p.m. Before the initiation of the study, subjects underwent 4 days of sucrose adaptation, during the light phase of the circadian cycle, followed by a baseline sucrose preference test, as described in EXAMPLE 8. On the next day, body weight was measured and the mice were injected (i.p., in a volume of 10 ml/kg) with the following treatments: Celecoxib (10, 20 or 30 mg/kg, Sigma, Israel), CBD (30 mg/kg, BOL, Israel), or Celecoxib plus CBD combinations (10:30, 20:30 or 30:30 mg/kg, respectively). Celecoxib, CBD and their combinations were dissolved in ethanol/cremophor/saline solution, at a ratio of 1:1:18. Control mice were injected with the vehicle only. The mice were similarly injected on the next two days (i.e., for a total of 3 consecutive daily injections). One hour following the third treatment injection, vehicle-treated mice were injected i.p. with either saline or 250 µg/kg LPS, while all the other groups were injected with the same dose of LPS. This LPS dose was used because we previously found that it induces substantial anxiolytic and depressive-like behavioral symptoms that lasts for at least 24 hours. We employed the one-hour time interval between the Celecoxib/CBD treatment and LPS injections based on the pharmacokinetic profile of CBD, which suggests that Tₘₐₓ occurs between 60 and 120 minutes in the brain and plasma following an i.p. injection in mice. Four hours following the LPS/Sal injection, the behavior of the mice was examined in the open field test, as described in Example 8. Sucrose preference was assessed, as described above, between 8 to 27 hr post LPS/Sal. Body weight and weight of the food were measured two hours before the LPS injection, and again after 24 hours, and the change in body weight and food consumption were calculated. All data are presented as mean ± SEM. Statistical comparisons were computed using SPSS software and consisted of one-way analyses of variance (ANOVA), followed by post-hoc comparisons with the Student-Newman-Keuls procedure.

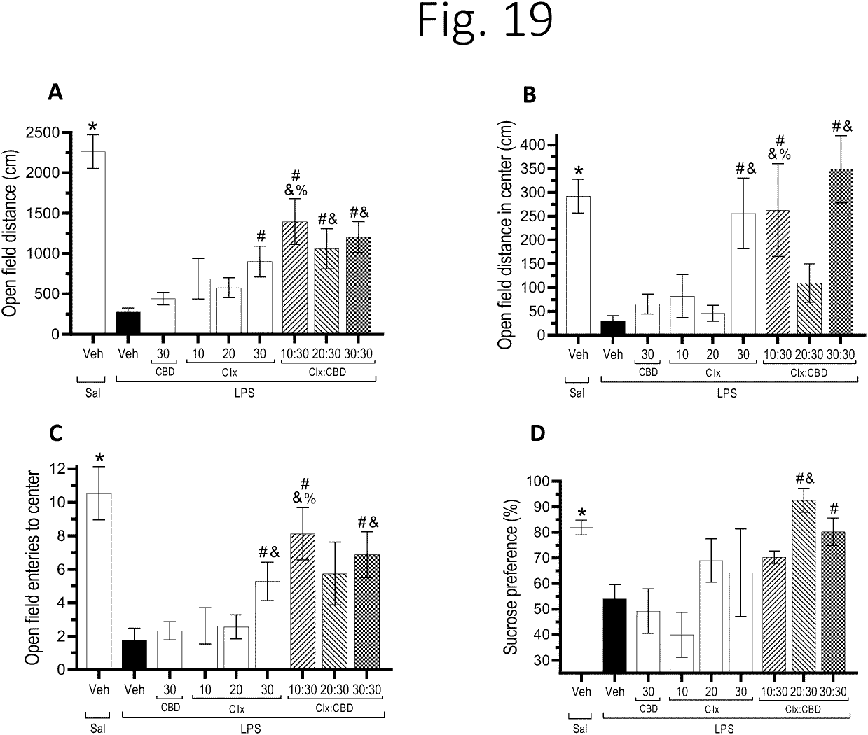

In the open field test, there was a significant difference between the groups in the distance moved (F_{18,83}=18.44, p<0.001) (FIG. 19A). Post-hoc tests comparing the vehicle-treated mice injected with saline vs. LPS revealed that LPS significantly reduced the distance travelled by the mice, i.e., their exploratory/locomotor activity. Treatment with CLX, at concentrations of 10 and 20 mg/kg or CBD by themselves did not have significant effects on the distance travelled, whereas celecoxib at 30 mg/kg did produce a significant increase in distance travelled compared with the vehicle-LPS group. Combinations of celecoxib plus CBD, at either 20:30 or 30:30 ratios significantly increased the distance travelled compared with the vehicle-LPS group. Furthermore, the effects of the combinations of celecoxib plus CBD, at either 20:30 or 30:30 ratios, were also significantly elevated compared with the CBD only group. Finally, the combination of celecoxib plus CBD at the 20:30 ratio, was also significantly different from the celecoxib at the corresponding dose (20 mg/kg), reflecting a potentiation effect.

Analysis of parameters related to behavior in the center of the open-field revealed significant differences between the groups in the distance moved in the center and the number of entries into the center area of the open field (F₈, ₈₅=2.46 and 9.65, p< 0.01, and 0.001, respectively) (FIGS. 19B-19C). Post-hoc analysis demonstrated that there were no significant effects of celecoxib or CBD by themselves, except for a significant increase in the number of entries following 30 mg/kg celecoxib (all compared with the vehicle-LPS group). Combinations of celecoxib plus CBD, at all ratios for the distance in center and entries, produced a significant elevation compared with the vehicle-LPS group. For the distance in center the combinations of 30:30 was different from the CBD group, and for the number of entries both the 10:30 and 30:30 combinations were different from the CBD group. Finally, the number of entries following treatment with the combination of celecoxib plus CBD at 10:30 ratio was significantly higher than the number in the celecoxib 10 mg/kg by itself.

In the Sucrose Preference test, there was a significant difference between the groups (F₈,₇₅=3.71, p<0.01) (FIG. 19D). Post-hoc analysis demonstrated that there were no significant effects of celecoxib or CBD by themselves. The combinations of celecoxib and CBD, at 20:30 and 30:30 ratios produced a significant increase in sucrose preference, compared with the vehicle-LPS group. The sucrose preference following treatment with the combination of celecoxib plus CBD at the 10:30 ratio was higher than the preference of the group treatment with 10 mg/kg celecoxib by itself.

These findings demonstrate that specific combinations of celecoxib with CBD, at doses that do not influence depression- or anxiety-related symptoms by themselves, can produce synergistic, dose-sparing therapeutic effects in a model of inflammation-associated depression.

### Example 19

### Therapeutic Effects of Celecoxib Plus CBD Combination on Behavioral Impairments in the Chronic Social Defeat Stress Paradigm

Exposure to repeated episodes of social defeat stress in mice induces a depression-like syndrome, characterized by anhedonia, anxiety and social-avoidance behaviors. In the current example, we assessed the effects of a combination of celecoxib and CBD on these measures. Given the expected severity of the behavioral symptoms, a relatively high doses of the two components of this combination (30 mg/kg from each) were used.

Subjects were 11-13 weeks old male C57BL/6 inbred mice and 4-6 months old CD-1 mice that were screened for high levels of aggressive behavior. Each CD-1 mouse was housed in one side of a social defeat cage, which had a clear perforated Plexiglas divider separating the two sides of the cage. Experimental C57Bl/6 mice were subjected to 5-10 minutes physical interaction sessions, once daily for 10 consecutive days, with a different CD-1 mouse each time. Following each interaction, the C57Bl/6 mouse was removed and placed in the contiguous empty side of the cage for the remining 24 hr. The control group comprised naïve, untreated mice that were kept undisturbed in their home cages. From day 6 to day 10 of the stress exposure paradigm, one group of experimental mice received an intraperitoneal (i.p.) injection of a combination of 30 mg/kg Celecoxib and 30 mg/kg CBD for five consecutive days. A second group of experimental mice (the Vehicle group) was injected with the vehicle solution only. The naïve control mice did not receive any treatment. Behavioral assessments in the open field test, elevated plus maze test, social exploration test and sucrose preference test were conducted from day 7 till day 11 post CSDS initiation as described in previous Examples. Following the last behavioral test, brain tissues were collected as previously described.

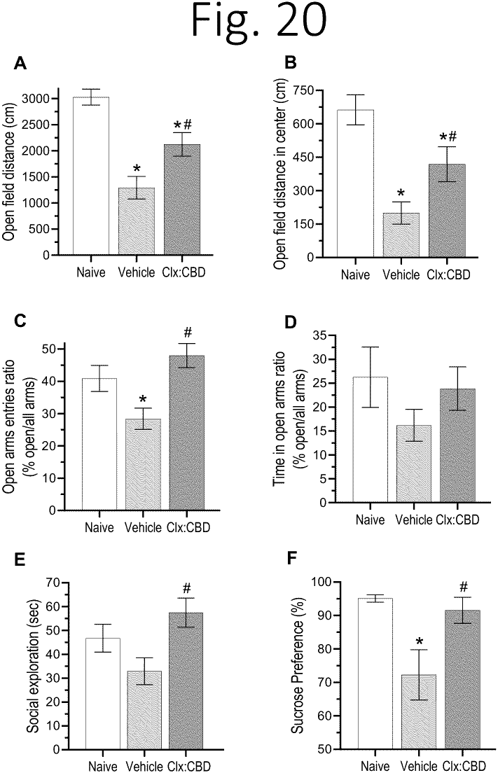

In the open field test, there was a significant difference between the groups in the distance moved (F_{2,33}=17.92, p<0.0001) (FIG. 20A). Post-hoc analyses revealed that this measure was significantly lower in the vehicle-treated group, compared with both the naïve group (p<0.0001*). In addition, the group treated with celecoxib plus CBD combination displayed a significant increase in this measurement when compared to the vehicle-rated group (p<0.01^{#}), but still a significant reduction when compared with the naïve group (p<0.005*). The distance moved in the center of the open field arena was significantly different between the groups (F_{2,34}=11.22, p<0.0005) (FIG. 20B). Post-hoc analyses revealed that this measure was significantly lower in the vehicle-treated group, when compared with the naïve group (p<0.0001*). In addition, the group treated with celecoxib plus CBD combination displayed a significant elevation in this measure when compared to vehicle-rated group (p<0.05^{#}), but still a significant reduction when compared to naïve group (p<0.05*). In the elevated plus maze test, there was a significant difference between the groups in the ratio of entries to the open arms (F₂,₃₂=6.289, P=0.005) (FIG. 20C). Post-hoc analyses revealed that this measure was significantly lower in the vehicle-treated group, when compared with the naïve group (p<0.05*). In addition, the group treated with celecoxib plus CBD combination displayed a significant increase in this measure when compared to vehicle-rated group (p<0.005^{#}), with no significant difference between the celecoxib plus CBD combination treatment group and the naïve group. The ratio of open arms time was not significantly different between the groups (FIG. 20D). In the social exploration test, there was a significant difference between the groups (F₂,₃₃= 4.192, p<0.05) (FIG. 20E). Post-hoc analysis revealed that mice in the celecoxib plus CBD combination treatment group displayed a significant increase in this measure, compared with vehicle-treated mice (p<0.01^{#}). Sucrose preference was significantly different between the groups (F_{2,} ₁₃= 8.880, p<0.005). Post-hoc analysis revealed that this measure was significantly lower in the vehicle-treated group, when compared with the naïve group (p<0.005*), and that celecoxib plus CBD combination treatment significantly increased this measure, compared with the vehicle-treated group (p<0.01^{#}). There was no significant difference between celecoxib plus CBD combination treatment group and the naïve group in this measurement.

These findings corroborate and extend the results of Example 15, by demonstrating that a combination of celecoxib plus CBD can not only influence inflammatory depression, but it is also effective in ameliorating the depression and anxiety symptoms elicited by chronic stress.

### Example 20

### Antidepressant and Anxiolytic Effects of Microglia-Suppressive Cannabis Related Formulation in Depressed Patients with a High Inflammatory Status

The antidepressant and anxiolytic effects of the most efficient microglia-suppressive formulation of cannabinoids or cannabinoid(s) with terpene(s) and/or flavonoid(s), or a specific whole plant cannabis extract, by itself or supplemented with cannabinoid(s) and/or terpene(s) and/or flavonoid(s), discovered in previous Examples is tested in a randomized, prospective, double-blind clinical trial in 60 treatment-resistant major depression patients of both genders, in the age range of 23-70 years. The safety and efficacy of this formulation (vs. vehicle placebo) are assessed. Primary endpoints include the changes from baseline to post-treatment in the vegetative and somatic symptoms of depression. Secondary endpoints include the changes from baseline to post-treatment in overall depression levels, as well as anxiety levels, overall functioning and clinical impression, and anhedonia.

Treatment-resistant depressed patients undergo psychiatric assessment using the Montgomery-Åsberg Depression Rating Scale (MADRS), with further analysis of the 3 specific factors of this scale (vegetative symptoms, dysphoria, and retardation), the Somatic Symptoms Scale-8, the Hamilton Anxiety Rating Scale (HARS), the Clinical Global Improvement and Severity Scales (CGI-I and CGI-S), and the Snaith-Hamilton Pleasure Scale (SHAPS). Subjects are treated twice daily (per os) with either 300 mg/kg of the formulation of cannabinoids or whole-plant cannabis extract, or cannabinoid(s) or whole-plant cannabis extract with terpene(s) and/or flavonoid(s), discovered in Examples 1-18 or with vehicle placebo. Blood samples are taken before treatment, as well as bi-weekly until the termination of the experiment after 8 weeks of treatment.

Plasma samples are used for analysis of CRP, and MINDD (Markers of Neuroinflammation in Neuropsychiatric Diseases), including CCL11 (eotaxin-1), IL-1β, TNFα, IL-6, IL-8, MIP-1α (CCL3), and MCP1 (CCL2) levels. Plasma MINDD levels are measured by specific ELISAs. In addition, intracellular MINDD levels in PBMCs isolated form the patients’ blood are examined by FACS.

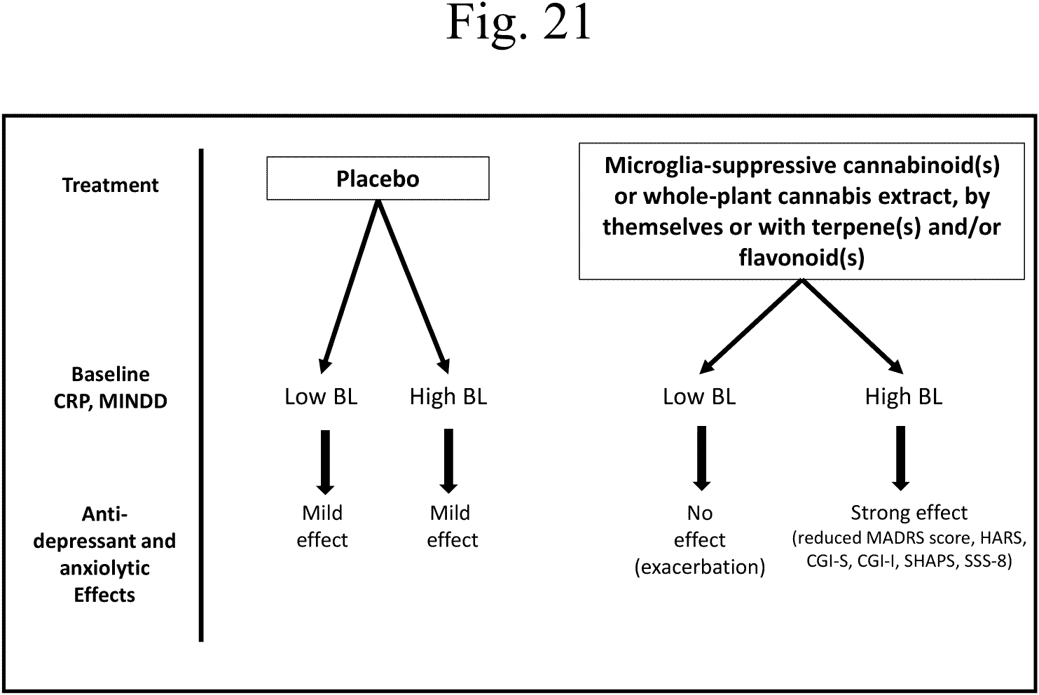

As presented in FIG. 21, the vehicle (placebo) treatment induces a mild beneficial effect on depression associated-vegetative and somatic symptoms, overall depression scores, anxiety, anhedonia, and subjective improvement in all subjects (irrespective of their inflammatory status levels). The formulation of cannabinoids or cannabinoid(s) with terpene(s) and/or flavonoid(s), or a specific whole plant cannabis extract, by itself or supplemented with cannabinoid(s) and/or terpene(s) and/or flavonoid(s), is highly efficacious in depressed patients with high baseline levels of CRP (greater than 1 mg/L) and/or other MINDD. These patients show significant reductions in the MADRS score, particularly in the vegetative factor of the MADRS, as well as in the somatic symptoms (SSS-8), and anxiety levels (measured by the HARS), along with improvements in the CGI and SHAPS (anhedonia) scores. In contrast, patients with low levels of CRP (lower than 1 mg/L) and/or other MINDD do not display any changes in depression, anxiety, anhedonia, and subjective improvement.

### Example 21

### Antidepressant and Anxiolytic Effects of Celecoxib Plus CBD Combination in Depressed Patients With High Levels of CRP and Other MINDD

The antidepressant effects of celecoxib were demonstrated both in animal models and in double-blind, randomized, placebo-controlled add-on trials. Participants in these trials were usually patients with either treatment-resistant depression or depression associated with another morbidity, such as osteoarthritis, brucellosis, fibromyalgia or cancer. A meta-analysis of data from 13 such clinical trials, demonstrated an overall significant antidepressant effect of celecoxib, although heterogeneity was high among studies was high, and the findings of about half of the studies did not reach statistical significance (Kohler-Forsberg et al., 2019). The celecoxib doses used in these studies were either 400 mg, once daily, or 200 mg, twice daily, for 6-8 weeks. Importantly, in one clinical study celecoxib was found to be efficacious in patients with high, but not low microglia activation status (measured by PET imaging of TSPO ligand that reflect the activation status of microglia). To date, there is almost no clinical studies on the antidepressant effects of CBD in humans, besides a recent report on successful treatment in a case series with three treatment-resistant depression patients. Furthermore, a survey of 3963 regular users of CBD for medical reasons, depression was the third reason stated by the participants for using CBD (after chronic pain and anxiety), with about half of the participants reporting that CBD treated their condition “Very Well or Moderately Well by Itself”. In experimental animals, extensive research demonstrated that both acute and chronic administration of CBD produced antidepressant effects in many models of depression, including depressive-like conditions associated with stress exposure, physical illness or genetic manipulations.

In the current study, the antidepressant effect of a combination of celecoxib and CBD is tested in a randomized, prospective, double-blind clinical trial in 80 treatment-resistant major depression patients of both genders, in the age range of 23-70 years. Subjects are randomly divided into 4 groups, treated with: 1) a formulation of 100 mg/kg celecoxib (i.e., a quarter of the usual dose of celecoxib that has been given in previous studies on depression), 2) 150 mg/kg CBD, 3) a combination of celecoxib (100 mg) and CBD (150 mg), and 4) placebo. These doses are translated from the findings in Example 15, which demonstrate a positive interaction between celecoxib and CBD at doses of 20 and 30 mg/kg, respectively. The human doses are computed by multiplying these values by 0.08 (as suggested by the FDA). The safety and efficacy of celecoxib, CBD and their combination vs. vehicle placebo are assessed. Primary endpoints include the changes from baseline to post-treatment in overall depression levels, and particularly in the vegetative and somatic symptoms of depression, which are highly associated with the inflammatory status of the patients. Secondary endpoints include the changes from baseline to post-treatment in anxiety levels, overall functioning and clinical impression, as well as anhedonia.

Treatment-resistant depressed patients undergo psychological assessment using the Montgomery-Asberg Depression Rating Scale (MADRS), with further analysis of the 3 specific factors of this scale (vegetative symptoms, dysphoria, and retardation), the Somatic Symptoms Scale-8, the Hamilton Anxiety Rating Scale (HARS), the Clinical Global Improvement and Severity Scales (CGI-I and CGI-S), and the Snaith-Hamilton Pleasure Scale (SHAPS). Subjects are treated twice daily (per os) with either a formulation containing celecoxib and CBD or with vehicle placebo. Blood samples are taken before treatment, as well as bi-weekly until the termination of the experiment after 8 weeks of treatment.

Plasma samples are used for analysis of CRP, and MINDD (Markers of Neuroinflammation in Neuropsychiatric Diseases), including CCL11 (eotaxin-1), IL-1β, TNFα, IL-6, IL-8, MIP-1α (CCL3), and MCP1 (CCL2) levels. Plasma MINDD levels are measured by specific ELISAs. In addition, intracellular MINDD levels in PBMCs isolated form the patients’ blood are examined by FACS.

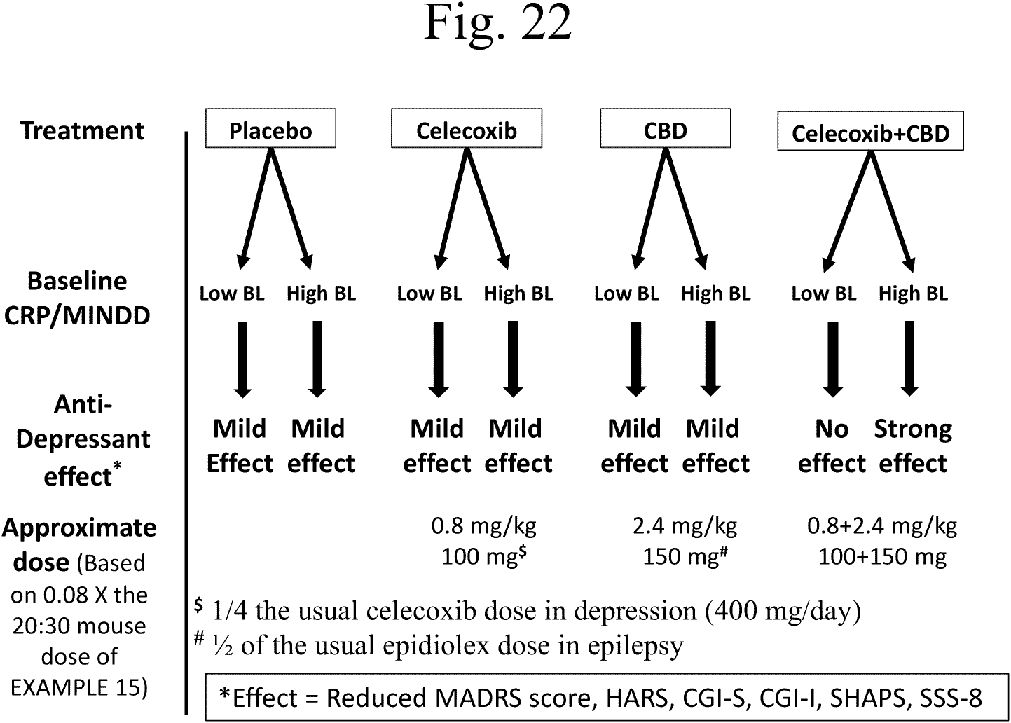

As presented in FIG. 22, the vehicle (placebo) treatment induces a mild beneficial effect on depression, in general, and on the associated-vegetative and somatic symptoms, in particular, as well as on anxiety, anhedonia and subjective improvement in all subjects (irrespective of their inflammatory status levels). The celecoxib plus CBD formulation is highly efficacious in depressed patients with high baseline levels of CRP (greater than 1 mg/L) and/or other MINDD. These patients show significant reductions in the MADRS score, particularly in the vegetative factor of the MADRS, as well as in the somatic symptoms (SSS-8), and anxiety levels (measured by the HARS), along with improvements in the CGI and SHAPS (anhedonia) scores. In contrast, patients with low levels of CRP (lower than 1 mg/L) and/or other MINDD do not display any changes in depression, anxiety, anhedonia and subjective improvement. Patients treated with placebo, or with celecoxib or CBD, each by itself, display only mild changes in depression, anxiety, anhedonia and subjective improvement (i.e., a placebo effect only).

In a separate clinical study, the antidepressant and anxiolytic effects of a combination of celecoxib and the most effective CBD-rich whole cannabis extract is tested in a randomized, prospective, double-blind clinical trial in 80 treatment-resistant major depression patients of both genders. The methodology of this experiment is identical to the above experiment, except that instead of CBD, subjects are treated with a CBD-rich whole cannabis extract, containing 150 mg CBD/day.

### Example 22

### Formulation of Cannabis-Related Molecules or Celecoxib Plus CBD Combination Improves Emotional and Cognitive Functioning in a Model of Parkinson’s Disease in Mice

Aggregated α-synuclein (αSyn), a major component of Lewy body is the pathological hallmark of PD, Lewy body dementia and other synucleinopathies, and unregulated αSyn expression in humans is linked to a higher risk of PD. α-Synuclein is particularly implicated in microglia activation and neuroinflammation that are causally associated with disease progression. Consistently, Thy1-aSyn mice, with genetically-induced over-expression of α-synuclein and clinical PD-like phenotype, exhibit elevated levels of peripheral immune markers, along with marked activation of microglia, particularly in the nigrostriatal system. The current invention demonstrates that in Thy1-aSyn mice, a formulation of celecoxib plus CBD produces synergistic potent microglia suppression, enhances cognitive and motor functioning, and ameliorates depressive-like symptoms.

Subjects are male and female 4 months-old Thy1-aSyn mice and their C57/BL littermate controls. Mice within each strain are divided into two subgroups (n=12, 6 males and 6 females) treated daily (via i.p. injections) for 6 weeks with either Vehicle (cremophor, ethanol and saline, v:v:v=1:1:18), or celecoxib (10 mg/kg), or CBD (5 mg/kg), or a combination of celecoxib (10 mg/kg) plus CBD (5 mg/kg). During the last two weeks of treatment, effects on emotional disturbances are assessed via the sucrose preference, social exploration, elevated plus maze and open-field tests, effects on cognitive functioning is assessed by the contextual fear conditioning, spatial recognition, and pattern separation tests, and motor function is measured via the beam traversal, pole descent, and hindlimb clasping reflexes. Mice are then sacrificed, peripheral blood is collected, and then the animals are perfused. Brains are prepared for immunohistochemistry. Microglia are stained by Iba-1 antibodies, and hippocampal microglial density and morphology (soma and processes size, processes number and length), as well as expression of activation markers (e.g., MHCII), is analyzed. Adult newborn neurons are stained with doublecortin (DCX), and their number is quantified as a measure of neurogenesis. The levels of α-synuclein are assessed by immunohistochemistry, particularly in the substantia nigra area, as well as by western blotting, using Mouse Anti-Human/Mouse/Rat α-synuclein Monoclonal Antibody (R&D Systems). The levels of plasma inflammatory markers, including CRP, CCL11 (eotaxin), IL-1β, TNFα, IL-8, MIP-1α (CCL3), and MCP1, are measured by ELIAS, compared between the groups and correlated with the behavioral and microglial variables.

Vehicle-treated Thy1-αSyn mice are found to display significant depression-like symptoms, along with cognitive and motor impairments, as well as greater activation of microglia (as compared with the corresponding WT controls). Thy1-αSyn mice treated with the formulation of cannabinoids or whole plant cannabis extract, by themselves or with terpene(s) and/or flavonoid(s) display lower levels of microglia activation, along with improved emotional, cognitive and motor functioning as compared with vehicle-treated Thy1-αSyn mice.

In a separate experiment, utilizing an identical experimental design, the effects of treatment with a combination of celecoxib and CBD are assessed. The experimental details are identical to the experiment mentioned above, except that subjects (male and female 4 months-old Thy1-aSyn mice and their C57/BL littermate controls) are treated daily (via i.p. injections) for 6 weeks with either Vehicle (cremophor, ethanol and saline, v:v:v=1:1:18), or celecoxib (10 mg/kg), or CBD (5 mg/kg), or a combination of celecoxib (10 mg/kg) plus CBD (5 mg/kg). Treatment with the formulation of Thy1-αSyn mice treated with the celecoxib plus CBD formulation (but not, or significantly less, by each component by itself) display lower levels of microglia activation, along with improved motor, cognitive and emotional functioning as compared with vehicle-treated Thy1-αSyn mice.

### Example 23

### Microglia-Suppressive Formulation of Cannabis-Related Molecules or Celecoxib Plus CBD Produces Therapeutic Effects on Depression, Anxiety and Cognitive Disturbances in Parkinson’s Disease Patients with the Diffuse/Malignant Subtype and a High Inflammatory Status

Clinical studies demonstrated that PD is a complex and heterogenous disorder with large variability in the presented phenotypes and progression of motor and non-motor symptoms. For example, some patients present with depression, cognitive impairment, orthostatic hypotension, indicating a diffuse/malignant (DM) subtype that is associated with the most rapid progression rate, while other patients present with a pure motor subtype, that progresses more slowly.

Heterogeneity and high variability are also observed with respect to neuroinflammation and the peripheral immune state of PD patients, with some patients exhibiting higher microglia activation status (measured by TSPO binding in PET studies) or elevated levels of plasma inflammatory markers, while others exhibit normal microglial activation state and cytokine levels. Importantly, the clinical and immune heterogeneity are linked, evidenced by the finding that PD patients with the DM subtype (some of which characterized by a mutation in the LRRK2 gene), but not patients with the pure motor subtype, exhibit high levels of pro-inflammatory markers (e.g., IL-8, MCP1 and MIP-1β). Consistently, inflammatory cytokine levels were found to be correlated with non-motor symptoms, e.g., depression and cognitive decline. These findings are consistent with the relative inefficacy of anti-inflammatory drugs in PD, proposing the need for a personalized medical approach, based on the microglial/inflammatory state of the individual patient.

In the current experiment, the therapeutic effect of the most effective formulation of cannabinoids or whole plant cannabis extract, by themselves, or with terpene(s) or flavonoid(s) is tested in a randomized, prospective, placebo-controlled, double-blind clinical trial in 60 patients with the DM subtype of PD. Safety and efficacy of the treatment on the progression of emotional, cognitive and motor impairments is evaluated over a three months period and the relation of this progression to baseline inflammatory biomarkers and clinical presentation is determined.

Subjects are 25-75 years-old patients diagnosed with idiopathic Parkinson’s disease. Disease stage, classified according to the modified Hoehn and Yahr (H&Y) scale, is 2.5 or less when patients are on treatment. Only patients with the DM subtype of PD are included, which are characterized by depression (MADRS score ≥17), cognitive impairment (MoCA score≤26), orthostatic hypotension, rapid eye movement sleep behavior disorder (RBD Questionnaire score ≥ 5 points), excessive daytime sleepiness (ESS score > 8), and fast progression of the disease. Individuals currently using recreational or medicinal cannabis or have been using cannabinoid-based medications within the 3 months prior to screening are excluded from the study. At baseline, patients undergo clinical assessment of their status on the following measures: Mood disturbances are evaluated using the depression assessed by the Montgomery-Asberg Depression Rating Scale (MADRS), the Snaith-Hamilton Pleasure Scale (SHAPS, for assessment of anhedonia), the Hamilton Anxiety Rating Scale (HARS), Cognitive disturbances are tested with the Montreal Cognitive Assessment (MoCA), severity of motor symptoms is assessed by part III of the Movement Disorder Society Unified Parkinson’s Disease Rating Scale (MDS-UPDRS-III), the presence of REM sleep behavior disorder, assessed by the REM Sleep Behavior Disorder Screening Questionnaire (RBD Questionnaire), excessive daytime sleepiness is assessed by the Epworth Sleepiness Scale (ESS) and Autonomic dysfunction is measured by the Scales for Outcomes in Parkinson’s Disease – Autonomic (SCOPA-AUT).

Blood samples are taken at baseline, and during monthly visits to the clinic. In addition to the tests mentioned above (MADRS, SHAPS, HARS, MoCA, MDS-UPDRS-III, RBD questionnaire, and ESS), patients are assessed with the PDQ-39 questionnaire, a measure of health status and quality of life (in the practically defined off-medication state), and the Clinical Global Impression – Severity scale (CGI-S) and Improvement scale (CGI-I), sleep quality, measured by the Pittsburgh Sleep Quality Index (PSQI).

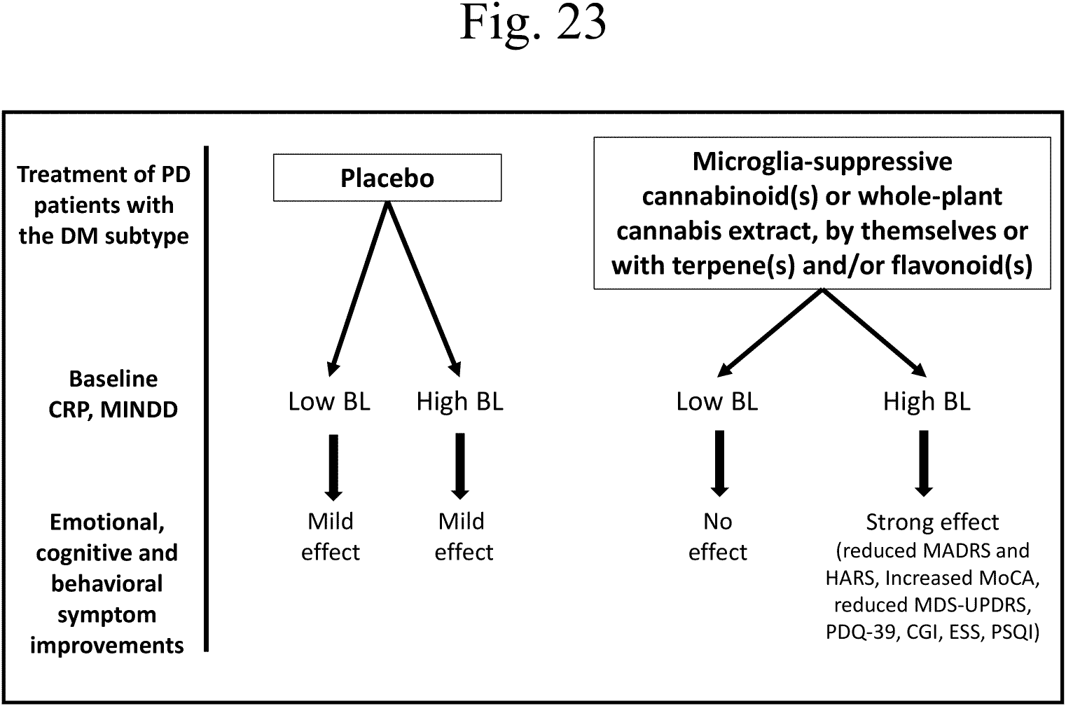

Patients with the diffuse/malignant subtype who have high levels of inflammatory markers (CRP, and/or IL-1β, and/or IL-6, and/or IL-8, and/or TNFα, and/or MIP-1α, and/or MCP-1, and/or MIP-1β, and/or RANTES), exhibit a strong beneficial effect. Specifically, significant reductions are found in the MADRS, SHAPS, and HARS scores, an increase is found in the MoCA test scores, along with decreases in the scores in the MDS-UPDRS-III, PDQ-39, RBD, ESS, CGI, PSQI and tests/questionnaires (FIG. 23). In contrast, patients treated with placebo display no changes in their emotional, cognitive, motor and behavioral symptoms, regardless of baseline inflammatory state.

In a separate experiment, utilizing an identical experimental design, the effects of treatment with a combination of celecoxib (100 mg) and CBD (150 mg; or a CBD-rich whole plant extract containing 150 mg CBD/day) is assessed. The experimental details are identical to the experiment mentioned above, except that subjects are treated daily with a formulation containing celecoxib (100 mg) plus CBD (150 mg), or each of these compounds by itself, or with placebo, as described in Example 21.

### Example 24

### Antidepressant Effects of Microglia-Suppressive Cannabinoid(s), and/or Terpene(s) and/or Flavonoid(s) Formulation in Depressed Alzheimer’s Disease Patients With High Levels of MINDD

Depression is a common symptom in patients with Alzheimer disease (AD), with a prevalence of more than 20%, causing distress, quality of life reduction, exacerbated cognitive and functional impairment, increased mortality, and increased stress and depression in caregivers. Furthermore, AD is associated with marked and dynamic alterations in microglia activation. Along the disease process, microglia undergo a series of changes, from beneficial, neuroprotective phenotype associated with phagocytosis and elimination of amyloid plaques, to hyper-inflammatory phenotype, associated with production of proinflammatory mediators, ROS leading to neurodegeneration. The inflammatory changes in the brain are paralleled by peripheral changes in MINDD, allowing the identification of AD patients suitable for treatment by the current invention.

The antidepressant effects of the most efficient microglia-suppressive formulation of cannabinoid(s) or cannabinoid(s) with terpene(s) and/or flavonoid(s) discovered in Examples 1-17 is tested in a randomized, prospective, double-blind clinical trial in 60 depressed AD patients of both sexes. Subjects are at least 50 years of age, of both sexes, with mild to moderate dementia (early and middle stages of Alzheimer’s disease), as measured by a Mini-Mental State Examination (MMSE) score lower than 23, a Clinical Dementia Rating (CDR) global score higher than 1 (above mild to moderate dementia), and a reliable informant or caregiver to accompany the patient to the clinic visits and to ensure taking the medication. Individuals currently using or have used recreational or medicinal cannabis or cannabinoid-based medications within the 3 months prior to screening and/or are unwilling to abstain for the duration of the trial, are excluded from the study. Eligible participants should have co-existing depression (≥4 weeks’ duration), assessed as potentially needing antidepressants. Depression severity is assessed using the Cornell Scale for Depression in Dementia (CSDD), with eligible participants scoring 8 or more.

The experiment is a three-month, randomized, double-blinded, placebo-controlled trial. Following screening, patients are randomly assigned to receive the composition of microglia-suppressive cannabinoid(s) or cannabinoid(s) with terpene(s) and/or flavonoids twice daily or placebo twice daily for 3 months. The composition and placebo are visually identical. Blood samples are taken at baseline, and during a monthly visit to the clinic, and CRP and MINDD levels are assessed at each time point.

The safety and efficacy of the formulation (vs. vehicle placebo), are assessed. The primary objective/end-point is the levels of depression, assessed by the Cornell Scale for Depression in Dementia (CSDD). Given that depression in AD patients can exacerbate cognitive functioning, secondary endpoints include the cognitive subscale of the AD Assessment Scale (ADAS-cog), the Clinician’s Interview Based Impression of Change with caregiver input (CIBIC+), the MMSE, Clinical Dementia Rating (CDR), the AD Cooperative Study Activities of Daily Living scale (ADCS-ADL). All tests are given at baseline, at monthly lab visits and at the completion of the study.

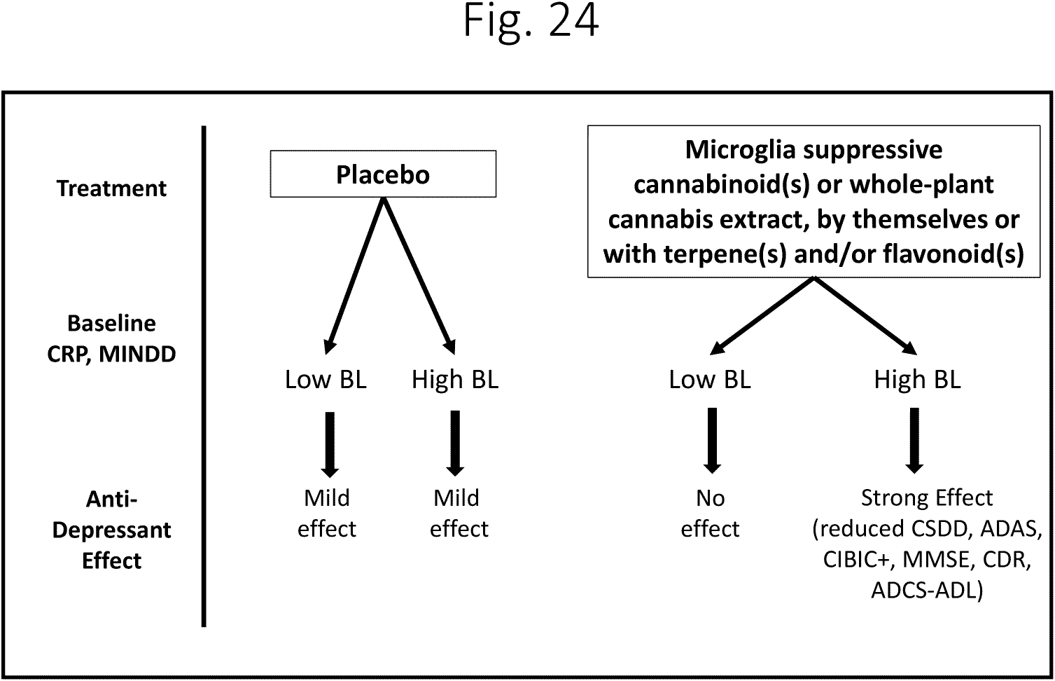

As presented in FIG. 24, depressed AD patients treated with vehicle (placebo), irrespective of their CRP and MINDD levels, exhibit a mild decline in depression, reflected by less than 3 points decline in the CSDD. Placebo-treated patients do not display significant improvements in the cognitive and behavioral functioning measures. The composition of cannabinoid(s) and/or cannabinoid(s) with terpene(s) and/or flavonoid(s) is efficacious in depressed AD patients with high baseline levels of CRP (greater than 1 mg/L), and/or other MINDD, including CCL11 (greater than 72 pg/ml), and/or IL-6 (greater than 2.0 pg/ml), and/or TNFα (greater than 3.8 pg/ml), and/or IL-8 (greater than 12 pg/ml), and/or MIP-1α (greater than 7.1 pg/ml), and/or MCP1 (greater than 80 pg/ml). These patients exhibit a significant decrease in the CSDD score, with more than 4 points decline. In addition, these patients show significant improvements in the ADAS-cog, CIBIC+, MMSE, CDR and ADCS-ADL. In contrast, depressed AD patients with low levels of CRP (lower than 1 mg/L) and/or other MINDD, including CCL11 (lower than 72 pg/ml), and/or IL-6 (lower than 2.0 pg/ml), and/or TNFα (lower than 3.8 pg/ml), and/or IL-8 (lower than 12 pg/ml), and/or MIP-1α (lower than 7.1 pg/ml), and/or MCP1 (lower than 80 pg/ml), and/or RANTES (lower than 2978 pg/ml), do not display improvements in any of these scales (FIG. 24).

### Example 25

### Effects of Various Cannabis Extracts on LPS -Induced Cytokine Production by BV2 Microglia

Purified, plant-derived cannabinoids, including CBD, were shown to exhibit anti-inflammatory and microglia-suppressive effects in various cellular models of inflammation and neuroinflammation. The modulation of microglial and inflammatory status by whole-plant cannabis extracts is more limited, but it has been suggested that the effects of such extracts may be superior to purified cannabinoids, due to the additive effects of other extract components including other (minor) cannabinoids, as well as terpenes, and flavonoids (comprising the “entourage effect”). Herein, we present an example of the beginning of a systematic investigation into the microglia-modulatory activity of various cannabis extract preparations on the activation of BV-2 microglial cells, using two CBD-enriched extracts (CannA and CannB, both with THC:CBD ratio of 1:20) or THC-enriched extract (CannC, with negligible concentration of CBD), in comparison with the effect of purified, plant-derived CBD.

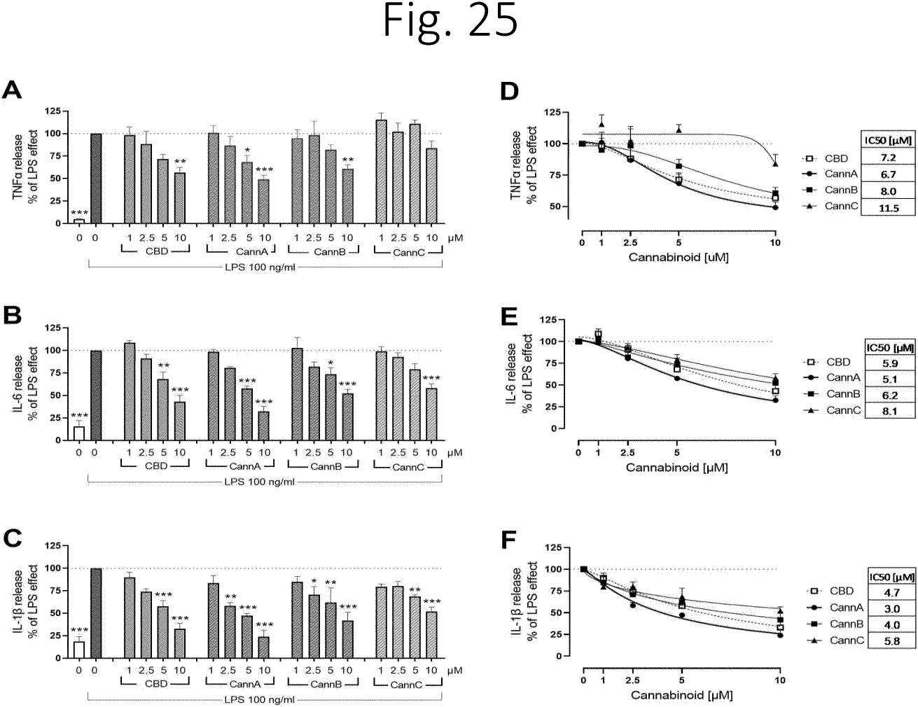

FIG. 25 depicts the effects of CBD, cannabis preparations including CannA and CannB (both CBD-enriched) and CannC (THC-enriched) on the release of TNFα, IL-6 and IL-1β by BV-2 microglial cells activated with 100 ng/ml of LPS. CBD or Cannabis extracts were all applied at the concentrations 1-10 µM, either of purified CBD or of the dominant cannabinoid in the extract (i.e., CBD or THC, calculated based on the known concentrations in the extracts). (FIG. 25A) Analysis of the effect of CBD and Cannabis extracts on the LPS-induced TNFα release revealed significant differences between the compounds (F_{17,32}=14.6; p<0.001. Post-hoc tests demonstrated that CannA effectively decreased TNFα release at concentrations of 5 µM (p<0.05) and 10 µM (p<0.001), whereas CBD and CannB significantly inhibited the release of TNFα only at 10 µM (p<0.01 for both). Neither of the tested concentrations of CannC extract (THC-enriched) significantly affected the release of TNFα, (FIG. 25D) The concentration-response calculations served to determine IC50s for TNFα inhibition, which were as follows: CBD IC50 = 7.2 µM, CannA IC50 = 6.7 µM, CannB IC50 = 8 µM and for CannC IC50 = 11.5 µM.(FIG. 25D) The applied compounds significantly suppressed IL-6 release (p<0.001; F_{17,32}=21.22; p<0.001). Post-hoc tests showed that the release of IL-6 was significantly decreased by 5 and 10 µM of CBD (p<0.01 and p<0.001, respectively), CannA (both p<0.001) and CannB (p<0.05 and p<0.001, respectively). Extract CannC applied at 10 µM also significantly inhibited the IL-6 protein levels (p<0.001). (FIG. 25E) The calculated IC50s were as follows: CBD 5.9 µM, CannA 5.1 µM, CannB 6.2 µM and for CannC was 8.1 µM.(FIG. 25C) The applied compounds significantly suppressed IL-1β release (F_{17,31} = 14.68; p<0.001). Post-hoc tests showed that concentration as low as 2.5 µM of CannA (p<0.01) and of CannB (p<0.05) already significantly decreased IL-1β release, and this inhibition was even stronger following 5 and 10 µM of CannA (both p<0.001) and of CannB (p<0.01 and p<0.001, respectively). CBD significantly reduced IL-1β release at 5 and 10 µM (p<0.001). Similarly, CannC extract also reduced IL-1β release when applied at 5 and 10 µM (p<0.01 and p<0.001, respectively. (FIG. 25F) The calculated IC50s were CBD 4.7 µM, CannA 3 µM, CannB 4.0 µM and for CannC was 5.8 µM.

These results exemplify the ability of specific CBD-enriched cannabis extracts to suppress microglia activation in a cellular model of neuroinflammation, with potency that is comparable or even stronger than purified CBD. For example, it is demonstrated that a specific CBD-enriched whole-plant extract (CannA) produces suppressive effects on TNFα and IL-1β release at lower concentrations of CBD than those exerted by the same concentrations of purified CBD, exemplifying the value of the entourage effect (which in the case of CannA contains the terpenes caryophyllene, terpinolene, myrcene, ocimene, guaiol, and pinene). The THC-enriched cannabis extract also exerted microglia-suppressive effects, albeit smaller than the other preparations tested.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A method for treating or attenuating a mood disorder in a subject in need thereof, the method comprising:
a determining whether said subject has a high inflammatory state and/or microglial activation state; and
b administering to said subject determined as having a high inflammatory state and/or microglial activation state a (i) a therapeutically effective amount of a first cannabinoid, and (ii) a therapeutically effective amount of at least one additional therapeutic compound,
thereby treating or attenuating a mood disorder in the subject.

2. The method of claim 1, wherein said high inflammatory/microglia state, is characterized by any one of: (a) plasma C-reactive protein (CRP) levels greater than 1 mg/L; (b) plasma IL-1b levels greater than 60 pg/ml; (c) plasma IL-6 levels greater than 2.0 pg/ml; (d) plasma TNFα levels greater than 3.8 pg/ml; (e) plasma CCL11 (eotaxin-1) levels greater than 72 pg/ml; (g) plasma IL-8 levels greater than 12.0 pg/ml; (h) plasma MIP-1α greater than 7.1 pg/ml; (i) plasma MCP1 levels greater than 80.0 pg/ml, (j) plasma RANTES greater than 2978 pg/ml, and any combination thereof.

3. The method of claim 1, wherein said determining is determining in a sample derived from said subject, and optionally wherein said method further comprises providing a sample from said subject and performing said determining in said sample.

4. (canceled)

5. The method of claim 1, wherein said high inflammatory state and/or microglial activation state comprises microglia activation, and optionally wherein said subject is resistant to standard medication for treatment of said condition.

6. (canceled)

7. The method of claim 1, wherein said mood disorder is selected from the group consisting of: major depressive disorder, unipolar major depressive episode, dysthymic disorder, treatment-resistant depression, bipolar depression, adjustment disorder with depressive mood, cyclothymic disorder, atypical depression, depression associated with REM sleep behavior disorder, seasonal affective disorder, depression co-morbid with anxiety disorder, generalized anxiety disorder, melancholic depression, psychotic depression, post-schizophrenic depression, schizophrenia, depression due to a general medical condition, depression associated with a synucleinopathy, Parkinson’s disease, multiple system atrophy (MSA), depression associated with autosomal-recessive Parkinson’s disease, depression associated with the diffuse/malignant subtype of Parkinson’s disease, depression associated with Alzheimer’s disease, depression associated with multiple sclerosis, depression associated with Tourette’s disorder, depression associated with dystonia, depression associated with ataxia, depression associated with dyskinesia, depression associated with essential tremor, depression associated with PTSD, post-viral fatigue syndrome, chronic fatigue syndrome, depression associated with autism spectrum disorder, depression associated with schizophrenia, depression associated with somatoform disorder, depression associated with somatic symptom disorder, depression associated with pain disorder, depression associated with rheumatoid arthritis, depression associated with osteoarthritis, depression associated with ankylosing spondylitis, depression associated with lupus erythematosus, depression associated with Crohn’s disease, depression associated with inflammatory bowel disease, depression associated with Williams syndrome, depression associated with the DiGeorge syndrome, depression associated with cancer, depression associated with primary biliary cholangitis, depression associated with autoimmune hepatitis, and depression associated with neurofibromatosis, and fibromyalgia.

8. The method of claim 1, wherein said at least one additional therapeutic compound is selected from the group consisting of: at least one cannabinoid being different from said first cannabinoid, a terpene, a flavonoid, and any combination thereof, optionally wherein said any one of first cannabinoid and said at least one cannabinoid being different from said first cannabinoid is selected from the group consisting of: cannabidiol (CBD), D-9-tetrahydrocannabinol (D-9-THC), D-8-tetrahydrocannabinol (D-8-THC), cannabigerol (CBG), cannabichromene (CBC), cannabigerolic acid (CBGA), Cannabidiolic acid (CBDA), THC acid (THCA), cannabichromenic Acid (CBCA), Cannabidivarin (CBDV), tetrahydrocannabivarin (THCV), Cannabichromevarin (CBCV), Cannabichromerocin (CBCV), Cannabivarin (CBV), Cannabicitran (CBT), Cannabinol (CBN), Cannabicyclol (CBL), Cannabigerorcin (CBGO), and Cannabinodiol (CBND), optionally wherein said first cannabinoid is CBD, optionally wherein said flavonoid is selected from the group consisting of: kaempferol, quercetin, cannflavin A, cannflavin B, canniprene, luteolin, apigenin, orientin, β-sitosterol, vitexin, isovitexin, and chrysin, and optionally wherein said terpene is selected from the group consisting of: b-caryophyllene, b-myrcene, linalool, α-pinene, b-pinene, limonene, b-amyrin, eucalyptol, alpha-terpineol, valencene, geraniol (lemonol), b-elemene, bisabolol, nerolidol, ocimene, terpinolene, humulene, α-terpinene, camphene, fenchol, α-phellandrene, Δ3-carene, g-cardinene, sabinene, and cycloartenol.

9-12. (canceled)

13. The method of claim 1, wherein said at least one additional therapeutic compound is an anti-inflammatory drug, an anti-microglial drug, or a combination thereof, optionally wherein said an anti-inflammatory drug is a non-steroidal anti-inflammatory drug (NSAID), optionally wherein said NSAID is celecoxib, and optionally wherein said at least one additional therapeutic compound is administered simultaneously or sequentially.

14-16. (canceled)

17. The method of claim 1, further comprising applying to said subject an anti-inflammatory or an anti-microglial therapy, and optionally wherein said anti-inflammatory therapy or said anti-microglial therapy comprises electroconvulsive therapy (ECT) .

18. (canceled)

19. A pharmaceutical composition comprising a first cannabinoid, at least one additional therapeutic compound, and a pharmaceutically acceptable carrier, for use in treating a mood disorder in a subject in need thereof, and being characterized by having microglia suppressive activity, optionally wherein said subject is determined as having a high inflammatory state and/or microglial activation state.

20. (canceled)

21. The pharmaceutical composition of claim 19, wherein said at least one additional therapeutic compound is selected from the group consisting of: at least one cannabinoid being different from said first cannabinoid, a terpene, a flavonoid, and any combination thereof, optionally wherein any one of said first cannabinoid and said at least one cannabinoid being different from said first cannabinoid is selected from the group consisting of: CBD, D-9-THC, D-8-THC, CBG, CBC, CBGA, CBDA, THCA, CBCA, CBDV, THCV, CBCV, CBCV, CBV, CBT, CBN, CBL, CBGO, and CBND, and optionally wherein said first cannabinoid is CBD.

22-23. (canceled)

24. The pharmaceutical composition of claim 21, wherein said flavonoid is selected from the group consisting of: kaempferol, quercetin, cannflavin A, cannflavin B, canniprene, luteolin, apigenin, orientin, β-sitosterol, vitexin, isovitexin, and chrysin, optionally wherein said terpene is selected from the group consisting of: b-caryophyllene, b-myrcene, linalool, α-pinene, b-pinene, limonene, b-amyrin, eucalyptol, alpha-terpineol, valencene, geraniol (lemonol), b-elemene, bisabolol, nerolidol, ocimene, terpinolene, humulene, α-terpinene, camphene, fenchol, α-phellandrene, Δ3-carene, g-cardinene, sabinene and cycloartenol.

25. (canceled)

26. The pharmaceutical composition of claim 19, wherein said at least one additional therapeutic compound is an anti-inflammatory drug, an anti-microglial drug, or a combination thereof, optionally wherein wherein said an anti-inflammatory drug is a non-steroidal anti-inflammatory drug (NSAID), and optionally wherein said NSAID is celecoxib.

27-28. (canceled)

29. The pharmaceutical composition of claim 24, comprising any one of: (i) CBD and kaempferol, at a ratio ranging from 4:1 to 1:4; (ii) CBD and quercetin, at a ratio ranging from 4:1 to 1:4; (iii) CBD and kaempferol, and quercetin, at a ratio ranging from 4:1:1 to 1:4:4; (iv) CBD and b-caryophyllene, and kaempferol, at a ratio ranging from 4:1:1 to 1:4:4; (v) CBD and b-caryophyllene, and quercetin, at a ratio ranging from 4:1:1 to 1:4:4; and (vi) any combination of (i) to (v).

30-33. (canceled)

34. The pharmaceutical composition of claim 21, comprising any one of: (i) CBD and THC, at a ratio ransging from 200:1 to 1:1; (ii) CBD, THC, and kaempferol, at a ratio ranging from 200:1:400 to 1:1:4; (iii) CBD, THC, and quercetin, at a ratio ranging from 200:1:400 to 1:1:4; and (iv) any combination of (i) to (iii).

35-36. (canceled)

37. The pharmaceutical composition of claim 26, comprising an anti-inflammatory drug, optionally wherein said anti-inflammatory drug is an NSAID, and optionally wherein said NSAID is selected from the group consisting of: celecoxib, aspirin, diclofenac, diflunisal, etodolac, ibuprofen, indomethacin, and acetaminophen.

38-39. (canceled)

40. The pharmaceutical composition of claim 26, comprising CBD and celecoxib, at a ratio ranging from 4:1 to 1:4.

41. The pharmaceutical composition of claim 21, comprising a whole plant cannabis extract comprising any one of: (i) CBD and THC at a ratio ranging from 30:1 (w/w) to 1:1 (w/w); (ii) CBD and THC at a ratio ranging from 30:1 (w/w) to 1:1 (w/w), and kaempferol; (iii) CBD and THC at a ratio ranging from 30:1 (w/w) to 1:1 (w/w), and quercetin; (iv) CBD and THC at a ratio ranging from 30:1 (w/w) to 1:1 (w/w), and b-caryophellene; (v) CBD and THC at a ratio ranging from 30:1 (w/w) to 1:1 (w/w) and b-caryophellene, and kaempferol, and quercetin; and (vi) any combination of (i) to (v).

42-45. (canceled)
